(19) United States
(12) Patent Application Publication
(10) Pub. No.: US 2022/0062379 A1
(43) Pub. Date: Mar. 3, 2022

(54) METHODS AND COMPOSITIONS FOR MODULATING IMMUNE DYSREGULATION

(71) Applicants: Luis BARREIRO, Montreal (CA); The General Hospital Corporation, Boston, MA (US); The Regents of the University of California, Oakland, CA (US)

(72) Inventors: Howland Shaw WARREN, Boston, MA (US); David GREGORY, Boston, MA (US); Wenzhong XIAO, Boston, CA (US); Feifei HAN, Boston, MA (US); Luis BARREIRO, Montreal (CA); Judith HELLMAN, Oakland, CA (US)

(21) Appl. No.: 17/423,244

(22) PCT Filed: Jan. 17, 2020
(86) PCT No.: PCT/US2020/014087

§ 371 Date: Jul. 15, 2021

Related U.S. Application Data
(60) Provisional application No. 62/930,813, filed on Nov. 5, 2019.
(60) Provisional application No. 62/794,386, filed on Jan. 18, 2019.

(51) Int. Cl. A61K 38/17; C12Q 1/6883; G01N 33/68; A61K 31/403; A61K 31/4965; A61K 31/4985; A61K 45/06

(52) U.S. Cl. CPC A61K 38/1709; C12Q 1/6883; G01N 33/6893; A61K 31/403; G01N 2800/24; A61K 31/4985; A61K 45/06; C12Q 2600/158; A61K 31/4965

(57) ABSTRACT

The invention relates to a method for treating a disease of immune dysregulation or altering an immune response or both in a mammal Such methods include administering to the mammal a therapeutically effective amount of a modulating agent or subjecting the mammal to a modulating process, wherein the modulating agent or process alters the expression or activity of a gene target associated with a sensitive response or a resistant response to an inflammatory stimulus.

### CROSS-REFERENCE TO RELATED APPLICATIONS

This application claims benefit of U.S. Provisional Application No. 62/794,386, filed Jan. 18, 2019, and U.S. Provisional Application No. 62/930,813, filed Nov. 5, 2019, the contents of which are incorporated herein by reference in their entirety.

### STATEMENT AS TO FEDERALLY FUNDED RESEARCH

This invention was supported under contract number W911 NF-16-C-0079 awarded by the Defense Advanced Research Projects Agency. The government has certain rights in the invention.

### SEQUENCE LISTING

The instant application contains a Sequence Listing which has been submitted electronically in ASCII format and is hereby incorporated by reference in its entirety. Said ASCII copy, created on Jan. 15, 2020, is named 51363-002W05_Sequence_Listing_1.15.20_ST25, and is 1,448,149 bytes in size.

### BACKGROUND OF THE INVENTION

Vertebrate species vary tremendously in their innate resistance (or tolerance) to inflammatory stimuli. The pathways, however, that underlie this resistance (or tolerance) are poorly understood. There is accordingly an unmet need in the art for methods that not only identify targets responsible for regulating inflammation, but also for identifying compounds and methods for treating inflammatory diseases.

### SUMMARY OF THE INVENTION

In general, the invention, in one aspect, features a method for treating a disease of immune dysregulation (e.g., the onset of the disease) in a mammal, the method including administering to the mammal a therapeutically effective amount of a modulating agent or subjecting the mammal to a modulating process, wherein the modulating agent or process alters the expression or activity of a gene target associated with a sensitive response or a resistant response to an inflammatory stimulus.

In another aspect, the invention features a method for altering an immune response in a mammal, the method including administering to the mammal a therapeutically effective amount of a modulating agent or subjecting the mammal to a modulating process, wherein the modulating agent or process alters the expression or activity of a gene target associated with a sensitive response or a resistant response to an inflammatory stimulus.

In either of these aspects, the gene target is a gene or gene product associated with a sensitive response. In some embodiments, the gene is one or more of the following EMC9, IRAK4, NFATC2, PLA2G10, SARM1, PIP5K1A, PDIA4, ARPC4, BNIP3, CCDC65, CENPH, CHPT1, COMMD3, DR1, FGD1, FIGNL1, GGNBP2, GNAO1, H2AFZ, HSPB1, IL17RB, IL17RC, ILF2, PDCD6, PI3, POFUT2, RABGEF1, RBM4, SKA2, SLC38A2, SNRPA1, SPCS2, TAF1D, TNFSF10, ZNF302, ZNF333, ZNF419, ZNF624, ZNF677, ZNF720, LPA, B4GAT1, CDH6, CUTA, DMBT1, FCGBP, OSMR, SSC5D, TNXB, BMP4, DPP4, MYLK2, MYLK4, ADRB1, ALDH1A2, ANKRD55, CLU, CTDSPL, CTNNAL1, DHFR, DNAJB4, DPYD, FZD10, GAB1, HCRTR1, IL7, LRRC1, MYLIP, NR1H3, PCGF5, PLSCR4, RAB11FIP1, RPGR, RUNX1T1, SLC40A1, SLCO2A1, STAT1, SULT1B1, TBC1D8, TGM2, or WNT4, or the gene product is an RNA or polypeptide encoded by the one or more of aforementioned genes. In other embodiments, the gene target is a gene or gene product associated with a resistant response. Preferably, the gene is one or more of the following ARHGEF10L, SLC8A1, TREML1, PEAR1, SFN, CD14, AOAH, ASPRV1, EHD1, LCN2, ST3GAL6, MFSD1, PECAM1, ESAM, AFM, APCS, F12, GCA, GLOD4, GP5, HGFAC, IGF1, MMRN2, PF4V1, PFKL, POSTN, SAA4, TIMP3, YWHAG, ADAM12, ADAM19, GADD45G, FGF1, RASD1 or RGS16 or the gene product is an RNA or polypeptide encoded by one or more of the aforementioned genes.

In other embodiments of these aspects, the modulating agent or process increases the expression or activity of the gene target. Accordingly, the modulating agent is an activator of the gene target, an agonist antibody of the gene target, a cell expressing the gene target, a mimetic of the gene target, a derivative or recombinant form of the gene target, or a soluble form of the gene target. In still other embodiments, the modulating process is overexpression of the gene target or overexpression or depletion of a signal, signaling regulator, or receptor associated with the gene target. In other embodiments, the modulating agent or process decreases the expression or activity of the gene target. And in other embodiments, the modulating agent is an inhibitor of the gene target, an inhibiting or neutralizing antibody of the gene target, a moiety blocking a receptor associated with the gene target, or a RNAi molecule targeting the gene target. Typically, the modulating process is depletion of cells expressing the gene target, overexpression or depletion of a signal, signaling regulator, or receptor associated with the gene target, depletion of a ligand of the gene target, or genetic ablation of the gene target. Exemplary modulating agents include a protein, a peptide, a polynucleotide, a small molecule, or a chemical (e.g., a gliptin such as saxagliptin, ISA-2011B, or benzamil).

In some embodiments, the modulating agent is delivered orally, by injection, by a lipid-based carrier, or by a nanoparticle-type carrier. In other embodiments, the modulating agent is delivered by an expression vector or plasmid containing a gene insert that codes for the immunomodulant agent. In other embodiments, the modulating agent is associated with a gene editing technology.

In some embodiments, the inflammatory stimulus is a bacterial lipopolysaccharide (LPS).

In some embodiments, the disease of immune dysregulation is an inflammatory disease. Exemplary inflammatory diseases include sepsis, achalasia, acute or ischemic colitis, acute respiratory distress syndrome, allergy, allograft rejection, alveolitis, Alzheimer's disease, a neurological disease associated with amyloidosis, amebiasis, anaphylactic shock, angiitis, ankylosing spondylitis, appendicitis, arteritis, arthralgia, arthritides, asthma, atherosclerosis, Behcet's syndrome, Berger's disease, bronchiolitis, bronchitis, burns, cachexia, candidiasis, cerebral embolism, cerebral infarction, cholangitis, cholecystitis, chronic fatigue syndrome, celiac disease, congestive heart failure, Crohn's disease, cystic fibrosis, Dengue fever, dermatitis, dermatomyositis, disseminated bacteremia, diverticulitis, duodenal ulcers, emphysema, encephalitis, endocarditis, endotoxic shock, enteritis, eosinophilic granuloma, epididymitis, epiglottitis, fasciitis, filariasis, gastric ulcers, Goodpasture's syndrome, gout, graft-versus-host disease, granulomatosis, Guillan-Barre syndrome, hay fever, hepatitis, hepatitis B virus infection, hepatitis C virus infection, herpes infection, HIV infection, Hodgkin's disease, hydatid cysts, hyperpyrexia, immune complex disease, influenza, malaria, meningitis, multiple sclerosis, a multiple sclerosis-associated demyelination disease, myasthenia gravis, myocardial ischemia, myocarditis, neuralgia, neuritis, organ ischemia, organ necrosis, osteomyelitis, Paget's disease, pancreatitis, ulcerative pancreatitis, pseudomembranous colitis, pancreatitis, paralysis, peptic ulcers, periarteritis nodosa, pericarditis, periodontal disease, peritonitis, pharyngitis, pleurisy, pneumonitis, pneumonoultramicroscopicsilicovolcanokoniosis, prostatitis, pseudomembranous Reiter's syndrome, reperfusion injury, respiratory syncytial virus infection, rheumatic fever, rheumatoid arthritis, rhinitis, sarcoidosis, septic abortion, septicemia, sinusitis, forms of cancer having an inflammatory component, spinal cord injury, sunburn, synovitis, systemic lupus erythematosus, systemic lupus erythrocytosis, thrombophlebitis, thyroiditis, Type I diabetes, ulcerative colitis, urethritis, urticaria, uveitis, vaginitis, vasculitis, warts, wheals, or Whipple's disease.

In other embodiments, the inflammatory disease is preferably a dermatological disorder such as one or more of the following: atopic dermatitis, alopecia areata, Bulloid pemphigus, eczema, chronic eczema, dermatomyositis, erythema nodosum, epidermolysis bullosa, hydradenitis suppurativa, lichen planus, pemphigus vulgaris, psoriasis, pyoderma gangrenosum, scleroderma, or vitiligo.

Preferably, the inflammatory disease is sepsis.

Preferred gastrointestinal disorders include Crohns' disease and ulcerative colitis.

Preferred neurological disorders include multiple sclerosis.

Preferred musculoskeletal disorders include ankylosing spondylitis, juvenile idiopathic arthritis, polymyalgia rheumatica, psoriatic arthritis, rheumatoid arthritis, systemic lupus erythematosus.

Preferred respiratory disorders include asthma and sarcoidosis.

In still other embodiments, the disease of immune dysregulation is secondary induced inflammation (for example, the secondary induced inflammation is associated with a bacterial infection, a viral infection, a fungal infection, or a parasitic infection (including bacterial and viral pneumonia, as well as microbial infection in all locations, including meningitis, pyelonephritis, sinusitis, etc.)).

In other embodiments, the inflammatory disease is allograft rejection and wherein the composition further includes an immunosuppressant used to inhibit allograft rejection.

In still other embodiments, the inflammatory disease is a xenograft rejection and wherein the composition further includes an immunosuppressant used to treat xenograft rejection.

In some embodiments of the aforementioned aspects, the mammal has a cancer and the alteration of the expression or activity of the gene target increases an innate immune response of the mammal (e.g., a human) or subject.

The invention further includes methods of screening to identify therapeutic interventions for treating a disorder of immune dysregulation. Accordingly, in another aspect, the invention features a method for identifying one or more effective therapeutic intervention targets for a disease of immune dysregulation, the method including: (i) measuring a whole transcriptome gene expression profile in leukocytes from a whole blood sample of a mammal, wherein the mammal or the whole blood sample has been treated with a pro-inflammatory stimulus or has not been treated with an inflammatory stimulus and wherein the mammal has an in vivo innate immune response that is resistant or sensitive, and (ii) identifying the whole-transcriptome gene expression profile as associated with innate immune resistance or sensitivity based on the in vivo innate immune response of the mammal, wherein the gene expression profiles are associated with innate immune resistance or sensitivity and are potential therapeutic targets for diseases of innate immune dysregulation.

In still another aspect, the invention features a method for identifying a therapeutic intervention target for a disease of immune dysregulation, the method including: (a) determining a gene expression profile in a blood sample of a first mammal, wherein the first mammal has an in vivo innate immune response that is resistant, (b) determining a gene expression profile in a blood sample of a second mammal, wherein the second mammal has an in vivo innate immune response that is sensitive, (c) identifying a gene or gene target having differential expression between the first mammal and the second mammal, and (d) identifying the gene or gene target as associated with a resistant response or a sensitive response based on the differential expression, wherein a gene or gene target associated with a resistant response or a sensitive response is identified as a therapeutic intervention target for a disease of immune dysregulation.

In either of these aspects, the first mammal and the second mammal, or the whole blood samples thereof, have not been treated with an inflammatory stimulus.

In some embodiments, the first mammal and the second mammal, or the whole blood samples thereof, have been treated with an inflammatory stimulus and the differential expression is differential expression following exposure to the inflammatory stimulus.

In some embodiments, the inflammatory stimulus is a toxin such as bacterial lipopolysaccharide (LPS) or a viral mimic (e.g., polyinosinic:polycytidylic acid (Poly(I:C))).

In other embodiments, the first mammal is one or more of baboon, rhesus monkey (rhesus macaque), rat, or mouse.

In still other embodiments, the second mammal is one or more of human, chimp, rabbit, sheep, cow, or pig.

The invention still further provides methods for assessing an immune response. Thus, in another aspect, the invention features a method for assessing an immune response of a mammal, the method including determining the expression of a gene target associated with a sensitive response or a resistant response to an inflammatory stimulus in the mammal, wherein the expression of the gene target identifies the immune response of the mammal as a sensitive response or a resistant response.

In some embodiments, the gene target is a gene or gene product associated with a resistant response. In some embodiments, the gene is one or more of ARHGEF10L, SLC8A1, TREML1, PEAR1, SFN, CD14, AOAH, ASPRV1, EHD1, LCN2, ST3GAL6, MFSD1, PECAM1, ESAM, AFM, APCS, F12, GCA, GLOD4, GP5, HGFAC, IGF1, MMRN2, PF4V1, PFKL, POSTN, SAA4, TIMP3, YWHAG, ADAM12, ADAM19, GADD45G, FGF1, RASD1 or RGS16, or the gene product is an RNA or polypeptide encoded by one or more of the aforementioned genes.

In some embodiments, the expression level of one or more (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35) of ARHGEF10L, SLC8A1, TREML1, PEAR1, SFN, CD14, AOAH, ASPRV1, EHD1, LCN2, ST3GAL6, MFSD1, PECAM1, ESAM, AFM, APCS, F12, GCA, GLOD4, GP5, HGFAC, IGF1, MMRN2, PF4V1, PFKL, POSTN, SAA4, TIMP3, YWHAG, ADAM12, ADAM19, GADD45G, FGF1, RASD1 or RGS16 is determined. In other embodiments, the expression level of one or more (e.g., 1, 2, 3, 4, 5, 6, 7, 8, 9, 10, 11, 12, 13, 14, 15, 16, or 17) of ARHGEF10L, SLC8A1, TREML1, PEAR1, SFN, CD14, AOAH, ASPRV1, EHD1, LCN2, ST3GAL6, MFSD1, PECAM1, ESAM, AFM, APCS, or F12 is determined.

In still other embodiments, the gene target is a gene or gene product associated with a sensitive response. For example, the gene target is gene is one or more of EMC9, IRAK4, NFATC2, PLA2G10, SARM1, PIP5K1A, PDIA4, ARPC4, BNIP3, CCDC65, CENPH, CHPT1, COMMD3, DR1, FGD1, FIGNL1, GGNBP2, GNAO1, H2AFZ, HSPB1, IL17RB, IL17RC, ILF2, PDCD6, PI3, POFUT2, RABGEF1, RBM4, SKA2, SLC38A2, SNRPA1, SPCS2, TAF1D, TNFSF10, ZNF302, ZNF333, ZNF419, ZNF624, ZNF677, ZNF720, LPA, B4GAT1, CDH6, CUTA, DMBT1, FCGBP, OSMR, SSC5D, TNXB, BMP4, DPP4, MYLK2, MYLK4, ADRB1, ALDH1A2, ANKRD55, CLU, CTDSPL, CTNNAL1, DHFR, DNAJB4, DPYD, FZD10, GAB1, HCRTR1, IL7, LRRC1, MYLIP, NR1H3, PCGF5, PLSCR4, RAB11FIP1, RPGR, RUNX1T1, SLC40A1, SLCO2A1, STAT1, SULT1B1, TBC1 D8, TGM2 or WNT4, or the gene product is an RNA or polypeptide encoded by one or more of the aforementioned genes.

In some embodiments, the expression level of one or more (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80 or 81) of EMC9, IRAK4, NFATC2, PLA2G10, SARM1, PIP5K1A, PDIA4, ARPC4, BNIP3, CCDC65, CENPH, CHPT1, COMMD3, DR1, FGD1, FIGNL1, GGNBP2, GNAO1, H2AFZ, HSPB1, IL17RB, IL17RC, ILF2, PDCD6, PI3, POFUT2, RABGEF1, RBM4, SKA2, SLC38A2, SNRPA1, SPCS2, TAF1D, TNFSF10, ZNF302, ZNF333, ZNF419, ZNF624, ZNF677, ZNF720, LPA, B4GAT1, CDH6, CUTA, DMBT1, FCGBP, OSMR, SSC5D, TNXB, BMP4, DPP4, MYLK2, MYLK4, ADRB1, ALDH1A2, ANKRD55, CLU, CTDSPL, CTNNAL1, DHFR, DNAJB4, DPYD, FZD10, GAB1, HCRTR1, IL7, LRRC1, MYLIP, NR1H3, PCGF5, PLSCR4, RAB11FIP1, RPGR, RUNX1T1, SLC40A1, SLCO2A1, STAT1, SULT1B1, TBC1 D8, TGM2 or WNT4 is determined. In other embodiments, the expression level of one or more (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, or 46) of EMC9, IRAK4, NFATC2, PLA2G10, SARM1, PIP5K1A, PDIA4, ARPC4, BNIP3, CCDC65, CENPH, CHPT1, COMMD3, DR1, FGD1, FIGNL1, GGNBP2, GNAO1, H2AFZ, HSPB1, IL17RB, IL17RC, ILF2, PDCD6, PI3, POFUT2, RABGEF1, RBM4, SKA2, SLC38A2, SNRPA1, SPCS2, TAF1 D, TNFSF10, ZNF302, ZNF333, ZNF419, ZNF624, ZNF677, ZNF720, LPA, B4GAT1, CDH6, CUTA, DMBT1, or FCGBP is determined. And still in other embodiments, the expression level of one or more (e.g., 1, 2, 3, 4, 5, 6, 7, 8, 9, 10, 11, 12, 13, 14, 15, 16, 17, 18, 19, or 20) of EMC9, IRAK4, NFATC2, PLA2G10, SARM1, PIP5K1A, PDIA4, ARPC4, BNIP3, CCDC65, CENPH, CHPT1, COMMD3, DR1, FGD1, FIGNL1, GGNBP2, GNAO1, H2AFZ, or HSPB1 is determined.

Typically, the expression level is an mRNA expression level which is determined. For example, the mRNA expression level is determined by PCR, RT-PCR, RNA-seq, gene expression profiling, serial analysis of gene expression, or microarray analysis. Preferably, the mRNA expression level is determined by RNA-seq.

In other embodiments, the expression level of a protein is determined. For example, the protein expression is determined by western blot, immunohistochemistry, or mass spectrometry.

In other aspects, the invention features methods of treating a disease of immune dysregulation as is disclosed herein. Thus, in another aspect, the invention features a method for treating a disease of immune dysregulation in a subject, the method including administering to the subject a therapeutically effective amount of a gliptin, benzamil, and/or ISA2011B. In some embodiments, the gliptin is vildagliptin, saxagliptin, alogliptin, linagliptin, sitagliptin, gemigliptin, anagliptin, and teneligliptin.

It is to be understood that aspects and embodiments of the invention described herein include “comprising,” “consisting,” and “consisting essentially of” aspects and embodiments. As used herein, the singular form “a,” “an,” and “the” includes plural references unless indicated otherwise.

As used herein, the term “treatment” (and variations thereof, such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease of immune dysregulation, alleviation of symptoms of such diseases, diminishment of any direct or indirect pathological consequences of the diseases, as well as altering an immune response. Additionally, treatment refers to clinical intervention relating to any of the diseases or conditions described herein.

As used herein, by the term “administering” is meant a method of giving a dosage of a compound to a subject. The compositions utilized in the methods described herein can be administered, for example, intravitreally (e.g., by intravitreal injection), by eye drop, intramuscularly, intravenously, intradermally, percutaneously, intraarterially, intraperitoneally, intralesionally, intracranially, intraarticularly, intraprostatically, intrapleurally, intratracheally, intrathecally, intranasally, intravaginally, intrarectally, topically, intratumorally, peritoneally, subcutaneously, subconjunctivally, intravesicularly, mucosally, intrapericardially, intraumbilically, intraocularly, intraorbitally, orally, topically, transdermally, by inhalation, by injection, by implantation, by infusion, by continuous infusion, by localized perfusion bathing target cells directly, by catheter, by lavage, in cremes, or in lipid compositions. The compositions utilized in the methods described herein can also be administered systemically or locally. For topical administration, a dosage is administered in a lotion, a cream, an ointment, or a gel. The method of administration can vary depending on various factors (e.g., the compound or composition being administered and the severity of the condition, disease, or disorder of immune dysregulation being treated).

A subject to be treated according to this invention is a mammal. The mammal could be, for example, a primate (e.g., a human), a rodent (e.g., a rat or a mouse), or a mammal of another species (e.g., farm or other domesticated animals) as is discussed herein. In each one of the above methods, the mammal may be one that suffers from an immunological disorder such as a disease of immune dysregulation.

A mammal “in need” of treatment can include, but are not limited to, mammals that have immunological disorders, mammals that have had immunological disorders, or mammals with symptoms of immunological disorders. Exemplary disorders are disclosed herein.

An “effective amount” of an agent, e.g., a pharmaceutical formulation, refers to an amount effective, at dosages and for periods of time necessary, to achieve the desired therapeutic or prophylactic result or a specifically state purpose. An “effective amount” can be determined empirically and by known methods relating to the stated purpose.

The terms “level of expression” or “expression level” in general are used interchangeably and generally refer to the amount of a target gene (or a biomarker) in a biological sample. “Expression” generally refers to the process by which information (e.g., gene-encoded and/or epigenetic information) is converted into the structures present and operating in the cell. Therefore, as used herein, “expression” may refer to transcription into a polynucleotide, translation into a polypeptide, or even polynucleotide and/or polypeptide modifications (e.g., posttranslational modification of a polypeptide). Fragments of the transcribed polynucleotide, the translated polypeptide, or polynucleotide and/or polypeptide modifications (e.g., post-translational modification of a polypeptide) shall also be regarded as expressed whether they originate from a transcript generated by alternative splicing or a degraded transcript, or from a post-translational processing of the polypeptide, e.g., by proteolysis. “Expressed genes” include those that are transcribed into a polynucleotide as mRNA and then translated into a polypeptide, and also those that are transcribed into RNA but not translated into a polypeptide (for example, transfer and ribosomal RNAs).

As used herein, the terms “disease of immune dysregulation” and “disease of innate immune dysregulation” refer to any disease, disorder, or condition associated with dysregulation of an immune response of a subject (e.g., a mammal). The dysregulation may be inappropriate immune activity (e.g., an overactive immune response) or lack of activity (e.g., an underactive immune response). Diseases of immune dysregulation include inflammatory diseases such as those diseases and conditions described herein.

As used herein, the term “secondary induced inflammation” refers to inflammation caused by an infection such as that caused by virtually any microbial infection or by non-infectious causes of inflammation. Such inflammation, for example, is present during infections involving viral or bacterial pneumonia, as well as in meningitis, pyelonephritis, and sinusitis. Some examples of non-infectious causes of secondary inflammation include pancreatitis, burn or trauma injury.

As used herein, the term “inflammatory stimulus” refers to a stimulus that may provoke an immune response in a subject (e.g., a mammal) or a sample (e.g., a cultured cell (e.g., a cultured endothelial cell) or a blood sample). In some examples, the inflammatory stimulus is an endotoxin (e.g., a bacterial lipopolysaccharide (LPS)) or a viral mimic (e.g., polyinosinic:polycytidylic acid (Poly(I:C)). In some embodiments, a microbial endotoxin provokes the immune response.

By a “toxin” is meant a compound produced by or part of an organism (e.g., a bacterium or another microbe) that results in an inflammatory response in a mammal or a subject. In one example, the toxin is a component of a bacterium for example, a gram-negative bacterium (in such cases, the toxin is a bacterial toxin. In some instances, the bacterial toxin may be secreted. Such secreted toxins include anthrax and pertussis toxins. In other examples, the toxin is lipopolysaccharide (LPS). In still other examples, the toxin is a viral or parasitic toxin.

As used herein, the terms “sensitive”, “sensitive response”, “innate sensitivity”, and “innate immune sensitivity” refer to an immune response of a species, subject, or sample (e.g., a cell culture or a blood sample) to an inflammatory stimulus (e.g., endotoxin challenge) that is sensitive relative to another group, e.g., has relatively high secondary inflammation following exposure to the inflammatory stimulus or is not able to tolerate more than a relatively low dose of the inflammatory stimulus before experiencing an adverse event, e.g., death.

As used herein, the terms “tolerant”, “resistant”, “resistant response”, “innate resistance”, and “innate immune resistance” refer to an immune response of a species, subject, or sample (e.g., a cell culture or a whole blood sample) to an inflammatory stimulus (e.g., endotoxin challenge) that is resistant or tolerant relative to another group, e.g., has relatively low secondary inflammation following the inflammatory stimulus or is able to tolerate a relatively high dose of the inflammatory stimulus before experiencing an adverse event, e.g., death.

As used herein, the term “gene target” refers to a gene or a gene product (e.g., an RNA, a polypeptide, or a protein) that is associated with a sensitive or a resistant response to an inflammatory stimulus and is the target of a modulating agent or a modulating process. Exemplary gene targets are described throughout the application. Preferred sensitive and resistant gene targets are respectively listed in Tables 6 and 7 herein.

A “patient” or “subject” herein refers to any single animal (including, e.g., a mammal, such as a dog, a cat, a horse, a rabbit, a zoo animal, a cow, a pig, a sheep, a non-human primate, and a human), such as a human, eligible for treatment who is experiencing or has experienced one or more signs, symptoms, or other indicators of a disease of immune dysregulation. Intended to be included as a patient are any patients involved in clinical research trials not showing any clinical sign of disease, patients involved in epidemiological studies, or patients once used as controls.

The term “modulating agent”, as used herein, refers to any moiety that can modulate the expression or activity of a gene target. In some examples, the modulating agent increases the expression or activity of a gene target. Exemplary modulating agents that may increase the expression or activity of a gene target (e.g., 5%, 10%, 20%, 30%, 40%, or 50% or greater relative to the reference level (e.g., the mean level) of a control) include, but are not limited to an activator of the gene target, an agonist antibody or antibody fragment of the gene target, a cell expressing the gene target, a mimetic of the gene target (including a mimetic that alters the expression or activity of the gene target), a derivative or recombinant form of the gene target, a soluble form of the gene target, or a moiety that alters the activity of an endogenous regulator of the gene target. In other examples, the modulating agent decreases the expression or activity of a gene target. Exemplary modulating agents that may decrease the expression or activity of a gene target (e.g., 5%, 10%, 20%, 30%, 40%, or 50% or greater relative to the reference level (e.g., the mean level) of a control) include, but are not limited to an inhibitor of the gene target, an inhibiting or neutralizing antibody of the gene target, a moiety blocking a receptor associated with the gene target, a moiety that alters the activity of an endogenous regulator of the gene target, or a RNAi molecule targeting the gene target.

The term “modulating process”, as used herein, refers to any process that can modulate the expression or activity of a gene target. In some examples, the modulating process increases the expression or activity of a gene target. Exemplary modulating processes that may increase the expression or activity of a gene target include, but are not limited to overexpression of the gene target and overexpression or depletion of a signal, signaling regulator, or receptor associated with the gene target. In other examples, the modulating process decreases the expression or activity of a gene target. Exemplary modulating processes that may decrease the expression or activity of a gene target include, but are not limited to depletion of cells expressing the gene target, overexpression or depletion of a signal, signaling regulator, or receptor associated with the gene target, depletion of a ligand of the gene target, or genetic ablation of the gene target.

As used herein, the term “altering an immune response” refers to a process in which a modulating agent or modulating process alters the expression or activity of a gene target related to inflammation in a subject (e.g., a mammal) or a sample (e.g., a cell culture or a blood sample) relative to subject or sample that has not been treated with the modulating agent. In some examples, the expression or activity of the gene target in the treated subject or sample is modified to resemble the expression or activity of the gene target in a species, subject, or sample having a resistant immune response, thus altering the immune response of the treated subject or sample to be more resistant. In other examples, the expression or activity of the gene target in the treated subject or sample is modified to resemble the expression or activity of the gene target in a species, subject, or sample having a sensitive immune response, thus altering the immune response of the treated subject or sample to be more sensitive.

Some embodiments of the technology and methodologies described herein can defined according to any of the following numbered paragraphs.

Treatment of a Disease of Immune Dysregulation
1. A method for treating the onset of a disease of immune dysregulation in a mammal, the method comprising administering to the mammal a therapeutically effective amount of a modulating agent or subjecting the mammal to a modulating process, wherein the modulating agent or process alters the expression or activity of a gene target associated with a sensitive response or a resistant response to an inflammatory stimulus.
2. A method for altering an immune response in a mammal, the method comprising administering to the mammal a therapeutically effective amount of a modulating agent or subjecting the mammal to a modulating process, wherein the modulating agent or process alters the expression or activity of a gene target associated with a sensitive response or a resistant response to an inflammatory stimulus.
3. The method of paragraph 1 or 2, wherein the gene target is a gene or gene product associated with a sensitive response (see, for example, Table 6).
4. The method of paragraph 3, wherein the gene is one or more of the following EMC9, IRAK4, NFATC2, PLA2G10, SARM1, PIP5K1A, PDIA4, ARPC4, BNIP3, CCDC65, CENPH, CHPT1, COMMD3, DR1, FGD1, FIGNL1, GGNBP2, GNAO1, H2AFZ, HSPB1, IL17RB, IL17RC, ILF2, PDCD6, PI3, POFUT2, RABGEF1, RBM4, SKA2, SLC38A2, SNRPA1, SPCS2, TAF1D, TNFSF10, ZNF302, ZNF333, ZNF419, ZNF624, ZNF677, ZNF720, LPA, B4GAT1, CDH6, CUTA, DMBT1, FCGBP, OSMR, SSC5D, TNXB, BMP4, DPP4, MYLK2, MYLK4, ADRB1, ALDH1A2, ANKRD55, CLU, CTDSPL, CTNNAL1, DHFR, DNAJB4, DPYD, FZD10, GAB1, HCRTR1, IL7, LRRC1, MYLIP, NR1H3, PCGF5, PLSCR4, RAB11FIP1, RPGR, RUNX1T1, SLC40A1, SLCO2A1, STAT1, SULT1B1, TBC1D8, TGM2, or WNT4, or the gene product is an RNA or polypeptide encoded by the one or more of aforementioned genes.
5. The method of paragraph 1 or 2, wherein the gene target is a gene or gene product associated with a resistant response (see, for example, Table 7).
6. The method of paragraph 5, wherein the gene is one or more of the following ARHGEF10L, SLC8A1, TREML1, PEAR1, SFN, CD14, AOAH, ASPRV1, EHD1, LCN2, ST3GAL6, MFSD1, PECAM1, ESAM, AFM, APCS, F12, GCA, GLOD4, GP5, HGFAC, IGF1, MMRN2, PF4V1, PFKL, POSTN, SAA4, TIMP3, YWHAG, ADAM12, ADAM19, GADD45G, FGF1, RASD1 or RGS16 or the gene product is an RNA or polypeptide encoded by one or more of the aforementioned genes.
7. The method of any one of paragraphs 1-6, wherein the modulating agent or process increases the expression or activity of the gene target.
8. The method of paragraph 7, wherein the modulating agent is an activator of the gene target, an agonist antibody of the gene target, a cell expressing the gene target, a mimetic of the gene target, a derivative or recombinant form of the gene target, or a soluble form of the gene target.
9. The method of paragraph 7, wherein the modulating process is overexpression of the gene target or overexpression or depletion of a signal, signaling regulator, or receptor associated with the gene target.
10. The method of any one of paragraphs 1-6, wherein the modulating agent or process decreases the expression or activity of the gene target.
11. The method of paragraph 10, wherein the modulating agent is an inhibitor of the gene target, an inhibiting or neutralizing antibody of the gene target, a moiety blocking a receptor associated with the gene target, or a RNAi molecule targeting the gene target.
12. The method of paragraph 10, wherein the modulating process is depletion of cells expressing the gene target, overexpression or depletion of a signal, signaling regulator, or receptor associated with the gene target, depletion of a ligand of the gene target, or genetic ablation of the gene target.
13. The method of any one of paragraphs 1-12, wherein the modulating agent is a protein, a peptide, a polynucleotide, a small molecule, or a chemical (e.g., a gliptin such as saxagliptin, ISA-2011B, or benzamil).
14. The method of any one of paragraphs 1-13, wherein the modulating agent is delivered orally, by injection, by a lipid-based carrier, or by a nanoparticle-type carrier.
15. The method of any one of paragraphs 1-13, wherein the modulating agent is delivered by an expression vector or plasmid containing a gene insert that codes for the immunomodulant agent.
16. The method of any one of paragraphs 1-13, wherein the modulating agent is associated with a gene editing technology.
17. The method of any one of paragraphs 1-16, wherein the inflammatory stimulus is a bacterial lipopolysaccharide (LPS).
18. The method of any one of paragraphs 1 and 3-17, wherein the disease of immune dysregulation is an inflammatory disease.
19. The method of paragraph 18, wherein the inflammatory disease is sepsis, achalasia, acute or ischemic colitis, acute respiratory distress syndrome, allergy, allograft rejection, alveolitis, Alzheimer's disease, a neurological disease associated with amyloidosis, amebiasis, anaphylactic shock, angiitis, ankylosing spondylitis, appendicitis, arteritis, arthralgia, arthritides, asthma, atherosclerosis, Behcet's syndrome, Berger's disease, bronchiolitis, bronchitis, burns, cachexia, candidiasis, cerebral embolism, cerebral infarction, cholangitis, cholecystitis, chronic fatigue syndrome, celiac disease, congestive heart failure, Crohn's disease, cystic fibrosis, Dengue fever, dermatitis, dermatomyositis, disseminated bacteremia, diverticulitis, duodenal ulcers, emphysema, encephalitis, endocarditis, endotoxic shock, enteritis, eosinophilic granuloma, epididymitis, epiglottitis, fasciitis, filariasis, gastric ulcers, Goodpasture's syndrome, gout, graft-versus-host disease, granulomatosis, Guillan-Barre syndrome, hay fever, hepatitis, hepatitis B virus infection, hepatitis C virus infection, herpes infection, HIV infection, Hodgkin's disease, hydatid cysts, hyperpyrexia, immune complex disease, influenza, malaria, meningitis, multiple sclerosis, a multiple sclerosis-associated demyelination disease, myasthenia gravis, myocardial ischemia, myocarditis, neuralgia, neuritis, organ ischemia, organ necrosis, osteomyelitis, Paget's disease, pancreatitis, ulcerative pancreatitis, pseudomembranous colitis, pancreatitis, paralysis, peptic ulcers, periarteritis nodosa, pericarditis, periodontal disease, peritonitis, pharyngitis, pleurisy, pneumonitis, pneumonoultramicroscopicsilicovolcanokoniosis, prostatitis, pseudomembranous Reiter's syndrome, reperfusion injury, respiratory syncytial virus infection, rheumatic fever, rheumatoid arthritis, rhinitis, sarcoidosis, septic abortion, septicemia, sinusitis, forms of cancer having an inflammatory component, spinal cord injury, sunburn, synovitis, systemic lupus erythematosus, systemic lupus erythrocytosis, thrombophlebitis, thyroiditis, Type I diabetes, ulcerative colitis, urethritis, urticaria, uveitis, vaginitis, vasculitis, warts, wheals, or Whipple's disease.
20. The method of paragraph 19, wherein the inflammatory disease is sepsis.
21. The method of any one of paragraphs 1 and 3-17, wherein the disease of immune dysregulation is secondary induced inflammation.
22. The method of paragraph 21, wherein the secondary induced inflammation is associated with a bacterial infection, a viral infection, a fungal infection, or a parasitic infection (including bacterial and viral pneumonia, as well as microbial infection in all locations, including meningitis, pyelonephritis, sinusitis, etc.)
23. The method of paragraph 19, wherein the inflammatory disease is allograft rejection and wherein the composition further comprises an immunosuppressant used to inhibit allograft rejection.
24. The method of paragraph 19, wherein the inflammatory disease is a xenograft rejection and wherein the composition further comprises an immunosuppressant used to treat xenograft rejection.
25. The method of paragraph 2, where the mammal has a cancer and the alteration of the expression or activity of the gene target increases an innate immune response of the mammal.
26. The method of any one of paragraphs 1-25, wherein the mammal is a human.

Screening for Therapeutic Intervention Targets
1. A method for identifying one or more effective therapeutic intervention targets for a disease of immune dysregulation, the method comprising:
   (i) measuring a whole transcriptome gene expression profile in leukocytes from a whole blood sample of a mammal, wherein the mammal or the whole blood sample has been treated with a pro-inflammatory stimulus or has not been treated with an inflammatory stimulus and wherein the mammal has an in vivo innate immune response that is resistant or sensitive, and
   (ii) identifying the whole-transcriptome gene expression profile as associated with innate immune resistance or sensitivity based on the in vivo innate immune response of the mammal,
      wherein the gene expression profiles are associated with innate immune resistance or sensitivity and are potential therapeutic targets for diseases of innate immune dysregulation.
2. A method for identifying a therapeutic intervention target for a disease of immune dysregulation, the method comprising:
   (a) determining a gene expression profile in a blood sample of a first mammal, wherein the first mammal has an in vivo innate immune response that is resistant,
   (b) determining a gene expression profile in a blood sample of a second mammal, wherein the second mammal has an in vivo innate immune response that is sensitive,
   (c) identifying a gene or gene target having differential expression between the first mammal and the second mammal, and
   (d) identifying said gene or gene target as associated with a resistant response or a sensitive response based on said differential expression, wherein a gene or gene target associated with a resistant response or a sensitive response is identified as a therapeutic intervention target for a disease of immune dysregulation.
3. The method of paragraph 2, wherein the first mammal and the second mammal, or the whole blood samples thereof, have not been treated with an inflammatory stimulus.
4. The method of paragraph 2, wherein the first mammal and the second mammal, or the whole blood samples thereof, have been treated with an inflammatory stimulus and the differential expression is differential expression following exposure to the inflammatory stimulus.
5. The method of paragraph 4, wherein the inflammatory stimulus is a toxin such as bacterial lipopolysaccharide (LPS) or a viral mimic.
6. The method of paragraph 4, wherein the viral mimic is polyinosinic:polycytidylic acid (Poly(I:C)).
7. The method of paragraph 2, wherein the first mammal is one or more of baboon, rhesus monkey (rhesus macaque), rat, or mouse.
8. The method of paragraph 2, wherein the second mammal is one or more of human, chimp, rabbit, sheep, cow, or pig.

Biomarkers of Sensitive or Resistant Responses
1. A method for assessing an immune response of a mammal, the method comprising determining the expression of a gene target associated with a sensitive response or a resistant response to an inflammatory stimulus in the mammal, wherein the expression of the gene target identifies the immune response of the mammal as a sensitive response or a resistant response.
2. The method of paragraph 1, wherein the gene target is a gene or gene product associated with a resistant response.
3. The method of paragraph 2, wherein the gene is one or more of ARHGEF10L, SLC8A1, TREML1, PEAR1, SFN, CD14, AOAH, ASPRV1, EHD1, LCN2, ST3GAL6, MFSD1, PECAM1, ESAM, AFM, APCS, F12, GCA, GLOD4, GP5, HGFAC, IGF1, MMRN2, PF4V1, PFKL, POSTN, SAA4, TIMP3, YWHAG, ADAM12, ADAM19, GADD45G, FGF1, RASD1 or RGS16, or the gene product is an RNA or polypeptide encoded by one or more of the aforementioned genes.
4. The method of paragraph 3, wherein the expression level of one or more (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35) of ARHGEF10L, SLC8A1, TREML1, PEAR1, SFN, CD14, AOAH, ASPRV1, EHD1, LCN2, ST3GAL6, MFSD1, PECAM1, ESAM, AFM, APCS, F12, GCA, GLOD4, GP5, HGFAC, IGF1, MMRN2, PF4V1, PFKL, POSTN, SAA4, TIMP3, YWHAG, ADAM12, ADAM19, GADD45G, FGF1, RASD1 or RGS16 is determined.
5. The method of paragraph 3, wherein the expression level of one or more (e.g., 1, 2, 3, 4, 5, 6, 7, 8, 9, 10, 11, 12, 13, 14, 15, 16, or 17) of ARHGEF10L, SLC8A1, TREML1, PEAR1, SFN, CD14, AOAH, ASPRV1, EHD1, LCN2, ST3GAL6, MFSD1, PECAM1, ESAM, AFM, APCS, or F12 is determined.
6. The method of paragraph 1, wherein the gene target is a gene or gene product associated with a sensitive response.
7. The method of paragraph 6, wherein the gene target is gene is one or more of EMC9, IRAK4, NFATC2, PLA2G10, SARM1, PIP5K1A, PDIA4, ARPC4, BNIP3, CCDC65, CENPH, CHPT1, COMMD3, DR1, FGD1, FIGNL1, GGNBP2, GNAO1, H2AFZ, HSPB1, IL17RB, IL17RC, ILF2, PDCD6, PI3, POFUT2, RABGEF1, RBM4, SKA2, SLC38A2, SNRPA1, SPCS2, TAF1D, TNFSF10, ZNF302, ZNF333, ZNF419, ZNF624, ZNF677, ZNF720, LPA, B4GAT1, CDH6, CUTA, DMBT1, FCGBP, OSMR, SSC5D, TNXB, BMP4, DPP4, MYLK2, MYLK4, ADRB1, ALDH1A2, ANKRD55, CLU, CTDSPL, CTNNAL1, DHFR, DNAJB4, DPYD, FZD10, GAB1, HCRTR1, IL7, LRRC1, MYLIP, NR1H3, PCGF5, PLSCR4, RAB11FIP1, RPGR, RUNX1T1, SLC40A1, SLCO2A1, STAT1, SULT1B1, TBC1D8, TGM2 or WNT4, or the gene product is an RNA or polypeptide encoded by one or more of the aforementioned genes.
8. The method of paragraph 7, wherein the expression level of one or more (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80 or 81) of EMC9, IRAK4, NFATC2, PLA2G10, SARM1, PIP5K1A, PDIA4, ARPC4, BNIP3, CCDC65, CENPH, CHPT1, COMMD3, DR1, FGD1, FIGNL1, GGNBP2, GNAO1, H2AFZ, HSPB1, IL17RB, IL17RC, ILF2, PDCD6, PI3, POFUT2, RABGEF1, RBM4, SKA2, SLC38A2, SNRPA1, SPCS2, TAF1D, TNFSF10, ZNF302, ZNF333, ZNF419, ZNF624, ZNF677, ZNF720, LPA, B4GAT1, CDH6, CUTA, DMBT1, FCGBP, OSMR, SSC5D, TNXB, BMP4, DPP4, MYLK2, MYLK4, ADRB1, ALDH1A2, ANKRD55, CLU, CTDSPL, CTNNAL1, DHFR, DNAJB4, DPYD, FZD10, GAB1, HCRTR1, IL7, LRRC1, MYLIP, NR1H3, PCGF5, PLSCR4, RAB11FIP1, RPGR, RUNX1T1, SLC40A1, SLCO2A1, STAT1, SULT1B1, TBC1D8, TGM2 or WNT4 is determined.
8. The method of paragraph 7, wherein the expression level of one or more (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, or 46) of EMC9, IRAK4, NFATC2, PLA2G10, SARM1, PIP5K1A, PDIA4, ARPC4, BNIP3, CCDC65, CENPH, CHPT1, COMMD3, DR1, FGD1, FIGNL1, GGNBP2, GNAO1, H2AFZ, HSPB1, IL17RB, IL17RC, ILF2, PDCD6, PI3, POFUT2, RABGEF1, RBM4, SKA2, SLC38A2, SNRPA1, SPCS2, TAF1D, TNFSF10, ZNF302, ZNF333, ZNF419, ZNF624, ZNF677, ZNF720, LPA, B4GAT1, CDH6, CUTA, DMBT1, or FCGBP is determined.
9. The method of paragraph 7, wherein the expression level of one or more (e.g., 1, 2, 3, 4, 5, 6, 7, 8, 9, 10, 11, 12, 13, 14, 15, 16, 17, 18, 19, or 20) of EMC9, IRAK4, NFATC2, PLA2G10, SARM1, PIP5K1A, PDIA4, ARPC4, BNIP3, CCDC65, CENPH, CHPT1, COMMD3, DR1, FGD1, FIGNL1, GGNBP2, GNAO1, H2AFZ, or HSPB1 is determined.
10. The method of paragraph 1, wherein the expression level is an mRNA expression level.
11. The method of paragraph 10, wherein the mRNA expression level is determined by PCR, RT-PCR, RNA-seq, gene expression profiling, serial analysis of gene expression, or microarray analysis.
12. The method of paragraph 11, wherein the mRNA expression level is determined by RNA-seq.
13. The method of paragraph 1, wherein the expression level is a protein expression level.
14. The method of paragraph 13, wherein the protein expression is determined by western blot, immunohistochemistry, or mass spectrometry.

Use of Targets for Treating Immune Dysregulation (e.g., Use of EMC9)
1. A method for treating the onset of a disease of immune dysregulation in a mammal, the method comprising administering to the mammal a therapeutically effective amount of a modulating agent or subjecting the mammal to a modulating process, wherein the modulating agent or process alters the expression or activity of EMC9 to an inflammatory stimulus.
2. A method for altering an immune response in a mammal, the method comprising administering to the mammal a therapeutically effective amount of a modulating agent or subjecting the mammal to a modulating process, wherein the modulating agent or process alters the expression or activity of EMC9 to an inflammatory stimulus.
3. The method of any one of paragraphs 1 or 2, wherein the modulating agent or process increases the expression or activity of EMC9.
4. The method of paragraph 3, wherein the modulating agent is an activator of EMC9, an agonist antibody of EMC9, a cell expressing EMC9, a mimetic of EMC9, a derivative or recombinant form of EMC9, or a soluble form of EMC9.
5. The method of paragraph 4, wherein the modulating process is overexpression of EMC9 or overexpression or depletion of a signal, signaling regulator, or receptor associated with EMC9.
6. The method of any one of paragraphs 1-5, wherein the modulating agent or process decreases the expression or activity of EMC9.
7. The method of paragraph 6, wherein the modulating agent is an inhibitor of EMC9, an inhibiting or neutralizing antibody of EMC9, a moiety blocking a receptor associated with the gene target, or a RNAi molecule targeting EMC9.
8. The method of paragraph 7, wherein the modulating process is depletion of cells expressing EMC9, overexpression or depletion of a signal, signaling regulator, or receptor associated with EMC9, depletion of a ligand of EMC9, or genetic ablation of EMC9.
9. The method of any one of paragraphs 1-8, wherein the modulating agent is a protein, a peptide, a polynucleotide, a small molecule, or a chemical.
10. The method of any one of paragraphs 1-9, wherein the modulating agent is delivered orally, by injection, by a lipid-based carrier, or by a nanoparticle-type carrier.
11. The method of any one of paragraphs 1-10, wherein the modulating agent is delivered by an expression vector or plasmid containing a gene insert that codes for the modulating agent.
12. The method of any one of paragraphs 1-11, wherein the modulating agent is associated with a gene editing technology.
13. The method of any one of paragraphs 1-12, wherein the inflammatory stimulus is a bacterial lipopolysaccharide (LPS).
14. The method of any one of paragraphs 1 and 3-13, wherein the disease of immune dysregulation is an inflammatory disease.
15. The method of paragraph 14, wherein the inflammatory disease is sepsis, achalasia, acute or ischemic colitis, acute respiratory distress syndrome, allergy, allograft rejection, alveolitis, Alzheimer's disease, a neurological disease associated with amyloidosis, amebiasis, anaphylactic shock, angiitis, ankylosing spondylitis, appendicitis, arteritis, arthralgia, arthritides, asthma, atherosclerosis, Behcet's syndrome, Berger's disease, bronchiolitis, bronchitis, burns, cachexia, candidiasis, cerebral embolism, cerebral infarction, cholangitis, cholecystitis, chronic fatigue syndrome, celiac disease, congestive heart failure, Crohn's disease, cystic fibrosis, Dengue fever, dermatitis, dermatomyositis, disseminated bacteremia, diverticulitis, duodenal ulcers, emphysema, encephalitis, endocarditis, endotoxic shock, enteritis, eosinophilic granuloma, epididymitis, epiglottitis, fasciitis, filariasis, gastric ulcers, Goodpasture's syndrome, gout, graft-versus-host disease, granulomatosis, Guillan-Barre syndrome, hay fever, hepatitis, hepatitis B virus infection, hepatitis C virus infection, herpes infection, HIV infection, Hodgkin's disease, hydatid cysts, hyperpyrexia, immune complex disease, influenza, malaria, meningitis, multiple sclerosis, a multiple sclerosis-associated demyelination disease, myasthenia gravis, myocardial ischemia, myocarditis, neuralgia, neuritis, organ ischemia, organ necrosis, osteomyelitis, Paget's disease, pancreatitis, ulcerative pancreatitis, pseudomembranous colitis, pancreatitis, paralysis, peptic ulcers, periarteritis nodosa, pericarditis, periodontal disease, peritonitis, pharyngitis, pleurisy, pneumonitis, pneumonoultramicroscopicsilicovolcanokoniosis, prostatitis, pseudomembranous Reiter's syndrome, reperfusion injury, respiratory syncytial virus infection, rheumatic fever, rheumatoid arthritis, rhinitis, sarcoidosis, septic abortion, septicemia, sinusitis, forms of cancer having an inflammatory component, spinal cord injury, sunburn, synovitis, systemic lupus erythematosus, systemic lupus erythrocytosis, thrombophlebitis, thyroiditis, Type I diabetes, ulcerative colitis, urethritis, urticaria, uveitis, vaginitis, vasculitis, warts, wheals, or Whipple's disease.
16. The method of paragraph 15, wherein the inflammatory disease is sepsis.
17. The method of any one of paragraphs 1 and 3-16, wherein the disease of immune dysregulation is secondary induced inflammation.
18. The method of paragraph 17, wherein the secondary induced inflammation is associated with a bacterial infection, a viral infection, a fungal infection, or a parasitic infection.
19. The method of paragraph 18, wherein the inflammatory disease is allograft rejection and wherein the composition further comprises an immunosuppressant used to inhibit allograft rejection.
20. The method of paragraph 19, wherein the inflammatory disease is a xenograft rejection and wherein the composition further comprises an immunosuppressant used to inhibit xenograft rejection.
21. The method of paragraph 2, where the mammal has a cancer and the alteration of the expression or activity of the gene target increases an innate immune response of the mammal
22. The method of any one of paragraphs 1-25, wherein the mammal is a human.

Gliptins, Benzamil, and ISA-2011B
1. A method for treating a disease of immune dysregulation in a subject, said method comprising administering to the subject a therapeutically effective amount of a gliptin, benzamil, or ISA2011B.
2. The method of paragraph 1, wherein the gliptin is vildagliptin, saxagliptin, alogliptin, linagliptin, sitagliptin, gemigliptin, anagliptin, and teneligliptin.
3. The method of paragraph 2, wherein saxagliptin is administered.
3. The method of paragraph 1, wherein benzamil is administered.
4. The method of paragraph 1, wherein ISA2011B is administered.
5. The method of paragraph 1, wherein the disease of immune dysregulation is sepsis, achalasia, acute or ischemic colitis, acute respiratory distress syndrome, allergy, allograft rejection, alveolitis, Alzheimer's disease, a neurological disease associated with amyloidosis, amebiasis, anaphylactic shock, angiitis, ankylosing spondylitis, appendicitis, arteritis, arthralgia, arthritides, asthma, atherosclerosis, Behcet's syndrome, Berger's disease, bronchiolitis, bronchitis, burns, cachexia, candidiasis, cerebral embolism, cerebral infarction, cholangitis, cholecystitis, chronic fatigue syndrome, celiac disease, congestive heart failure, Crohn's disease, cystic fibrosis, Dengue fever, dermatitis, dermatomyositis, disseminated bacteremia, diverticulitis, duodenal ulcers, emphysema, encephalitis, endocarditis, endotoxic shock, enteritis, eosinophilic granuloma, epididymitis, epiglottitis, fasciitis, filariasis, gastric ulcers, Goodpasture's syndrome, gout, graft-versus-host disease, granulomatosis, Guillan-Barre syndrome, hay fever, hepatitis, hepatitis B virus infection, hepatitis C virus infection, herpes infection, HIV infection, Hodgkin's disease, hydatid cysts, hyperpyrexia, immune complex disease, influenza, malaria, meningitis, multiple sclerosis, a multiple sclerosis-associated demyelination disease, myasthenia gravis, myocardial ischemia, myocarditis, neuralgia, neuritis, organ ischemia, organ necrosis, osteomyelitis, Paget's disease, pancreatitis, ulcerative pancreatitis, pseudomembranous colitis, pancreatitis, paralysis, peptic ulcers, periarteritis nodosa, pericarditis, periodontal disease, peritonitis, pharyngitis, pleurisy, pneumonitis, pneumonoultramicroscopicsilicovolcanokoniosis, prostatitis, pseudomembranous Reiter's syndrome, reperfusion injury, respiratory syncytial virus infection, rheumatic fever, rheumatoid arthritis, rhinitis, sarcoidosis, septic abortion, septicemia, sinusitis, forms of cancer having an inflammatory component, spinal cord injury, sunburn, synovitis, systemic lupus erythematosus, systemic lupus erythrocytosis, thrombophlebitis, thyroiditis, Type I diabetes, ulcerative colitis, urethritis, urticaria, uveitis, vaginitis, vasculitis, warts, wheals, or Whipple's disease.

Other features and advantages of the invention will be apparent from the following Detailed Description and the Claims.

### BRIEF DESCRIPTION OF THE DRAWINGS

This application file contains at least one drawing executed in color. Copies of this patent application with color drawings will be provided by the Office upon request and payment of the necessary fee.

FIG. 1A shows IRAK4 expression in 10 species with and without stimulation for various times. Resistant species are baboon, Rhesus monkey, rat and mouse; sensitive species are human, chimp, rabbit, sheep, cow, and pig; baseline conditions are at the far left.

FIG. 1B shows siRNA mediated knockdown of IRAK4 expression inhibits TNF release from human PBMCs to a similar extent as the LPS receptor TLR4. Data from 5 independent experiments using different human donors is plotted.

FIG. 1C shows IRAK4 kinase inhibitors block TNF release from whole blood stimulated with LPS. Representative of 2 experiments. This data confirms prior published work.

FIG. 2 shows TREML1 expression in 10 species with and without stimulation for various times. Resistant species are baboon, Rhesus monkey, rat and mouse; sensitive species are human, chimp, rabbit, sheep, cow, and pig; baseline conditions are at the far left.

FIG. 3 shows NFATC2 expression in 10 species with and without stimulation for various times. Resistant species are baboon, Rhesus monkey, rat and mouse; sensitive species are human, chimp, rabbit, sheep, cow, and pig; baseline conditions are at the far left.

FIG. 4 shows PLA2G10 expression in 10 species with and without stimulation for various times. Resistant species are baboon, Rhesus monkey, rat and mouse; sensitive species are human, chimp, rabbit, sheep, cow, and pig; baseline conditions are at the far left.

FIG. 5A shows SARM1 expression in 10 species with and without stimulation for various times. Resistant species are baboon, Rhesus monkey, rat and mouse; sensitive species are human, chimp, rabbit, sheep, cow, and pig; baseline conditions are at the far left.

FIG. 5B shows siRNA mediated knockdown of SARM1 expression inhibits TNF release from human PBMCs to a similar extent as the LPS receptor TLR4. Data from 5 independent experiments using different human donors is plotted.

FIG. 6 shows PIP5K1A expression in 10 species with and without stimulation for various times. Resistant species are baboon, Rhesus monkey, rat and mouse; sensitive species are human, chimp, rabbit, sheep, cow, and pig; baseline conditions are at the far left.

FIG. 7 shows PDIA4 expression in 10 species with and without stimulation for various times. Resistant species are baboon, Rhesus monkey, rat and mouse; sensitive species are human, chimp, rabbit, sheep, cow, and pig; baseline conditions are at the far left.

FIG. 8 shows ARPC4 expression in 10 species with and without stimulation for various times. Resistant species are baboon, Rhesus monkey, rat and mouse; sensitive species are human, chimp, rabbit, sheep, cow, and pig; baseline conditions are at the far left.

FIG. 9 shows BNIP3 expression in 10 species with and without stimulation for various times. Resistant species are baboon, Rhesus monkey, rat and mouse; sensitive species are human, chimp, rabbit, sheep, cow, and pig; baseline conditions are at the far left.

FIG. 10 shows CCDC65 expression in 10 species with and without stimulation for various times. Resistant species are baboon, Rhesus monkey, rat, and mouse; sensitive species are human, chimp, rabbit, sheep, cow, and pig; baseline conditions are at the far left.

FIG. 11 shows CENPH expression in 10 species with and without stimulation for various times. Resistant species are baboon, Rhesus monkey, rat, and mouse; sensitive species are human, chimp, rabbit, sheep, cow, and pig; baseline conditions are at the far left.

FIG. 12 shows CHPT1 expression in 10 species with and without stimulation for various times. Resistant species are baboon, Rhesus monkey, rat, and mouse; sensitive species are human, chimp, rabbit, sheep, cow, and pig; baseline conditions are at the far left.

FIG. 13 shows COMMD3 expression in 10 species with and without stimulation for various times. Resistant species are baboon, Rhesus monkey, rat, and mouse; sensitive species are human, chimp, rabbit, sheep, cow, and pig; baseline conditions are at the far left.

FIG. 14 shows DR1 expression in 10 species with and without stimulation for various times. Resistant species are baboon, Rhesus monkey, rat, and mouse; sensitive species are human, chimp, rabbit, sheep, cow, and pig; baseline conditions are at the far left.

FIG. 15 shows EMC9 expression in 10 species with and without stimulation for various times. Resistant species are baboon, Rhesus monkey, rat, and mouse; sensitive species are human, chimp, rabbit, sheep, cow, and pig; baseline conditions are at the far left.

FIG. 16 shows FGD1 expression in 10 species with and without stimulation for various times. Resistant species are baboon, Rhesus monkey, rat, and mouse; sensitive species are human, chimp, rabbit, sheep, cow, and pig; baseline conditions are at the far left.

FIG. 17 shows FIGNL1 expression in 10 species with and without stimulation for various times. Resistant species are baboon, Rhesus monkey, rat, and mouse; sensitive species are human, chimp, rabbit, sheep, cow, and pig; baseline conditions are at the far left.

FIG. 18 shows GGNBP2 expression in 10 species with and without stimulation for various times. Resistant species are baboon, Rhesus monkey, rat, and mouse; sensitive species are human, chimp, rabbit, sheep, cow, and pig; baseline conditions are at the far left.

FIG. 19 shows GNAO1 expression in 10 species with and without stimulation for various times. Resistant species are baboon, Rhesus monkey, rat, and mouse; sensitive species are human, chimp, rabbit, sheep, cow, and pig; baseline conditions are at the far left.

FIG. 20 shows H2AFZ expression in 10 species with and without stimulation for various times. Resistant species are baboon, Rhesus monkey, rat, and mouse; sensitive species are human, chimp, rabbit, sheep, cow, and pig; baseline conditions are at the far left.

FIG. 21 shows HSPB1 expression in 10 species with and without stimulation for various times. Resistant species are baboon, Rhesus monkey, rat, and mouse; sensitive species are human, chimp, rabbit, sheep, cow, and pig; baseline conditions are at the far left.

FIG. 22 shows IL17RB expression in 10 species with and without stimulation for various times. Resistant species are baboon, Rhesus monkey, rat, and mouse; sensitive species are human, chimp, rabbit, sheep, cow, and pig; baseline conditions are at the far left.

FIG. 23 shows IL17RC expression in 10 species with and without stimulation for various times. Resistant species are baboon, Rhesus monkey, rat, and mouse; sensitive species are human, chimp, rabbit, sheep, cow, and pig; baseline conditions are at the far left.

FIG. 24 shows ILF2 expression in 10 species with and without stimulation for various times. Resistant species are baboon, Rhesus monkey, rat, and mouse; sensitive species are human, chimp, rabbit, sheep, cow, and pig; baseline conditions are at the far left.

FIG. 25 shows PDCD6 expression in 10 species with and without stimulation for various times. Resistant species are baboon, Rhesus monkey, rat, and mouse; sensitive species are human, chimp, rabbit, sheep, cow, and pig; baseline conditions are at the far left.

FIG. 26 shows PEAR1 expression in 10 species with and without stimulation for various times. Resistant species are baboon, Rhesus monkey, rat, and mouse; sensitive species are human, chimp, rabbit, sheep, cow, and pig; baseline conditions are at the far left.

FIG. 27 shows PI3 expression in 10 species with and without stimulation for various times. Resistant species are baboon, Rhesus monkey, rat, and mouse; sensitive species are human, chimp, rabbit, sheep, cow, and pig; baseline conditions are at the far left.

FIG. 28 shows POFUT2 expression in 10 species with and without stimulation for various times. Resistant species are baboon, Rhesus monkey, rat, and mouse; sensitive species are human, chimp, rabbit, sheep, cow, and pig; baseline conditions are at the far left.

FIG. 29 shows RABGEF1 expression in 10 species with and without stimulation for various times. Resistant species are baboon, Rhesus monkey, rat, and mouse; sensitive species are human, chimp, rabbit, sheep, cow, and pig; baseline conditions are at the far left.

FIG. 30 shows RBM4 expression in 10 species with and without stimulation for various times. Resistant species are baboon, Rhesus monkey, rat, and mouse; sensitive species are human, chimp, rabbit, sheep, cow, and pig; baseline conditions are at the far left.

FIG. 31 shows SFN expression in 10 species with and without stimulation for various times Resistant species are baboon, Rhesus monkey, rat, and mouse; sensitive species are human, chimp, rabbit, sheep, cow, and pig; baseline conditions are at the far left.

FIG. 32 shows SKA2 expression in 10 species with and without stimulation for various times. Resistant species are baboon, Rhesus monkey, rat, and mouse; sensitive species are human, chimp, rabbit, sheep, cow, and pig; baseline conditions are at the far left.

FIG. 33 shows SLC38A2 expression in 10 species with and without stimulation for various times. Resistant species are baboon, Rhesus monkey, rat, and mouse; sensitive species are human, chimp, rabbit, sheep, cow, and pig; baseline conditions are at the far left.

FIG. 34 shows SNRPA1 expression in 10 species with and without stimulation for various times. Resistant species are baboon, Rhesus monkey, rat, and mouse; sensitive species are human, chimp, rabbit, sheep, cow, and pig; baseline conditions are at the far left.

FIG. 35 shows SPCS2 expression in 10 species with and without stimulation for various times. Resistant species are baboon, Rhesus monkey, rat, and mouse; sensitive species are human, chimp, rabbit, sheep, cow, and pig; baseline conditions are at the far left.

FIG. 36 shows TAF1D expression in 10 species with and without stimulation for various times. Resistant species are baboon, Rhesus monkey, rat, and mouse; sensitive species are human, chimp, rabbit, sheep, cow, and pig; baseline conditions are at the far left.

FIG. 37 shows TNFSF10 expression in 10 species with and without stimulation for various times. Resistant species are baboon, Rhesus monkey, rat, and mouse; sensitive species are human, chimp, rabbit, sheep, cow, and pig; baseline conditions are at the far left.

FIG. 38 shows ZNF302 expression in 10 species with and without stimulation for various times. Resistant species are baboon, Rhesus monkey, rat, and mouse; sensitive species are human, chimp, rabbit, sheep, cow, and pig; baseline conditions are at the far left.

FIG. 39 shows ZNF333 expression in 10 species with and without stimulation for various times. Resistant species are baboon, Rhesus monkey, rat, and mouse; sensitive species are human, chimp, rabbit, sheep, cow, and pig; baseline conditions are at the far left.

FIG. 40 shows ZNF419 expression in 10 species with and without stimulation for various times. Resistant species are baboon, Rhesus monkey, rat, and mouse; sensitive species are human, chimp, rabbit, sheep, cow, and pig; baseline conditions are at the far left.

FIG. 41 shows ZNF624 expression in 10 species with and without stimulation for various times. Resistant species are baboon, Rhesus monkey, rat, and mouse; sensitive species are human, chimp, rabbit, sheep, cow, and pig; baseline conditions are at the far left.

FIG. 42 shows ZNF677 expression in 10 species with and without stimulation for various times. Resistant species are baboon, Rhesus monkey, rat, and mouse; sensitive species are human, chimp, rabbit, sheep, cow, and pig; baseline conditions are at the far left.

FIG. 43 shows ZNF720 expression in 10 species with and without stimulation for various times. Resistant species are baboon, Rhesus monkey, rat, and mouse; sensitive species are human, chimp, rabbit, sheep, cow, and pig; baseline conditions are at the far left.

FIG. 44 shows the fold change on LPS stimulation in CD14 expression after 2 hours. Resistant species are baboon, Rhesus monkey, rat, and mouse; sensitive species are human, chimp, rabbit, sheep, cow, and pig.

FIG. 45 shows the fold change on LPS stimulation in AOAH expression after 24 hours. Resistant species are baboon, Rhesus monkey, rat, and mouse; sensitive species are human, chimp, rabbit, sheep, cow, and pig.

FIG. 46 shows the fold change on LPS stimulation in SLC8A1 expression after 6 hours. Resistant species are baboon, Rhesus monkey, rat, and mouse; sensitive species are human, chimp, rabbit, sheep, cow, and pig.

FIG. 47 shows the fold change on LPS stimulation in ASPRV1 expression after 6 hours. Resistant species are baboon, Rhesus monkey, rat, and mouse; sensitive species are human, chimp, rabbit, sheep, cow, and pig.

FIG. 48 shows the fold change on LPS stimulation in ARHGEF10L expression after 6 hours. Resistant species are baboon, Rhesus monkey, rat, and mouse; sensitive species are human, chimp, rabbit, sheep, cow, and pig.

FIG. 49 shows the fold change on LPS stimulation in EHD1 expression after 6 hours. Resistant species are baboon, Rhesus monkey, rat, and mouse; sensitive species are human, chimp, rabbit, sheep, cow, and pig.

FIG. 50 shows the fold change on LPS stimulation in LCN2 expression after 2 hours. Resistant species are baboon, Rhesus monkey, rat, and mouse; sensitive species are human, chimp, rabbit, sheep, cow, and pig.

FIG. 51 shows the fold change on LPS stimulation in LCN2 expression after 6 hours. Resistant species are baboon, Rhesus monkey, rat, and mouse; sensitive species are human, chimp, rabbit, sheep, cow, and pig.

FIG. 52 shows the fold change on LPS stimulation in ST3GAL6 expression after 6 hours Resistant species are baboon, Rhesus monkey, rat, and mouse; sensitive species are human, chimp, rabbit, sheep, cow, and pig.

FIG. 53 shows the fold change on LPS stimulation in MFSD1 expression after 24 hours. Resistant species are baboon, Rhesus monkey, rat, and mouse; sensitive species are human, chimp, rabbit, sheep, cow, and pig.

FIG. 54 shows the protein abundance of LPA. Resistant species are baboon, Rhesus monkey, mouse, and rat; sensitive species are human, chimp, rabbit, cow, pig, and sheep.

FIG. 55 shows the protein abundance of PECAM1. Resistant species are baboon, Rhesus monkey, mouse, and rat; sensitive species are human, chimp, rabbit, cow, pig, and sheep.

FIG. 56 shows the protein abundance of ESAM. Resistant species are baboon, Rhesus monkey, mouse, and rat; sensitive species are human, chimp, rabbit, cow, pig, and sheep.

FIG. 57 shows the protein abundance of AFM. Resistant species are baboon, Rhesus monkey, mouse, and rat; sensitive species are human, chimp, rabbit, cow, pig, and sheep.

FIG. 58 shows the protein abundance of APCS. Resistant species are baboon, Rhesus monkey, mouse, and rat; sensitive species are human, chimp, rabbit, cow, pig, and sheep.

FIG. 59 shows the protein abundance of B4GAT1. Resistant species are baboon, Rhesus monkey, mouse, and rat; sensitive species are human, chimp, rabbit, cow, pig, and sheep.

FIG. 60 shows the protein abundance of CDH6. Resistant species are baboon, Rhesus monkey, mouse, and rat; sensitive species are human, chimp, rabbit, cow, pig, and sheep.

FIG. 61 shows the protein abundance of CUTA. Resistant species are baboon, Rhesus monkey, mouse, and rat; sensitive species are human, chimp, rabbit, cow, pig, and sheep.

FIG. 62 shows the protein abundance of DMBT1. Resistant species are baboon, Rhesus monkey, mouse, and rat; sensitive species are human, chimp, rabbit, cow, pig, and sheep.

FIG. 63 shows the protein abundance of F12. Resistant species are baboon, Rhesus monkey, mouse, and rat; sensitive species are human, chimp, rabbit, cow, pig, and sheep.

FIG. 64 shows the protein abundance of FCGBP. Resistant species are baboon, Rhesus monkey, mouse, and rat; sensitive species are human, chimp, rabbit, cow, pig, and sheep.

FIG. 65 shows the protein abundance of GCA. Resistant species are baboon, Rhesus monkey, mouse, and rat; sensitive species are human, chimp, rabbit, cow, pig, and sheep.

FIG. 66 shows the protein abundance of GLOD4. Resistant species are baboon, Rhesus monkey, mouse, and rat; sensitive species are human, chimp, rabbit, cow, pig, and sheep.

FIG. 67 shows the protein abundance of GP5. Resistant species are baboon, Rhesus monkey, mouse, and rat; sensitive species are human, chimp, rabbit, cow, pig, and sheep.

FIG. 68 shows the protein abundance of HGFAC. Resistant species are baboon, Rhesus monkey, mouse, and rat; sensitive species are human, chimp, rabbit, cow, pig, and sheep.

FIG. 69 shows the protein abundance of IGF1. Resistant species are baboon, Rhesus monkey, mouse, and rat; sensitive species are human, chimp, rabbit, cow, pig, and sheep.

FIG. 70 shows the protein abundance of MMRN2 Resistant species are baboon, Rhesus monkey, mouse, and rat; sensitive species are human, chimp, rabbit, cow, pig, and sheep.

FIG. 71 shows the protein abundance of OSMR. Resistant species are baboon, Rhesus monkey, mouse, and rat; sensitive species are human, chimp, rabbit, cow, pig, and sheep.

FIG. 72 shows the protein abundance of PF4V1. Resistant species are baboon, Rhesus monkey, mouse, and rat; sensitive species are human, chimp, rabbit, cow, pig, and sheep.

FIG. 73 shows the protein abundance of PFKL. Resistant species are baboon, Rhesus monkey, mouse, and rat; sensitive species are human, chimp, rabbit, cow, pig, and sheep.

FIG. 74 shows the protein abundance of POSTN. Resistant species are baboon, Rhesus monkey, mouse, and rat; sensitive species are human, chimp, rabbit, cow, pig, and sheep.

FIG. 75 shows the protein abundance of SAA4. Resistant species are baboon, Rhesus monkey, mouse, and rat; sensitive species are human, chimp, rabbit, cow, pig, and sheep.

FIG. 76 shows the protein abundance of SSC5D. Resistant species are baboon, Rhesus monkey, mouse, and rat; sensitive species are human, chimp, rabbit, cow, pig, and sheep.

FIG. 77 shows the protein abundance of TIMP3. Resistant species are baboon, Rhesus monkey, mouse, and rat; sensitive species are human, chimp, rabbit, cow, pig, and sheep.

FIG. 78 shows the protein abundance of TNXB. Resistant species are baboon, Rhesus monkey, mouse, and rat; sensitive species are human, chimp, rabbit, cow, pig, and sheep.

FIG. 79 shows the protein abundance of YWHAG. Resistant species are baboon, Rhesus monkey, mouse, and rat; sensitive species are human, chimp, rabbit, cow, pig, and sheep.

FIG. 80A shows DPP4 expression in endothelial cells from different species. Resistant species are rat and mouse; sensitive species are cow and human.

FIG. 80B shows DPP4 expression in human endothelial cells cultured in serum from different species. Resistant species are mouse and rat, sensitive species is human.

FIG. 80C shows ECIS plots demonstrating that saxagliptin, a DPP4 inhibitor, causes a dose dependent reduction in human endothelial cell barrier permeability following HKEC stimulation.

FIG. 81A shows BMP4 expression in endothelial cells from different species. Resistant species are colored are rat and mouse, sensitive species are cow and human.

FIG. 81B shows BMP4 expression in human endothelial cells cultured in serum from different species. Resistant species are mouse and rat, sensitive species is human.

FIG. 81C shows proinflammatory cytokine secretion from whole blood treated with and without LPS and with and without SJ00291942, an agonist of BMP receptor signaling.

FIG. 82A shows MYLK2 expression of human endothelial cells pretreated with HKEC (tolerant) and without pre-treatment prior to to the indicated stimulations.

FIG. 82B shows MYLK2 expression in endothelial cells from different species. Resistant species are rat and mouse, sensitive species is human.

FIG. 82C shows MYLK4 expression of human endothelial cells pretreated with HKEC (tolerant) and without pre-treatment prior to the indicated stimulations.

FIG. 82D shows MYLK4 expression in endothelial cells from different species. Resistant species are rat and mouse, sensitive species is human.

FIG. 83A shows ADAM12 expression in endothelial cells from different species. Resistant species are rat and mouse, sensitive species are cow and human.

FIG. 83B shows ADAM12 expression in human endothelial cells cultured in serum from different species. Resistant species are rat and mouse, sensitive species is human.

FIG. 83C shows ADAM19 expression in endothelial cells from different species. Resistant species are rat and mouse, sensitive species is human.

FIG. 83D shows ADAM19 expression in human endothelial cells cultured in serum from different species. Resistant species are mouse and rat, sensitive species is human.

FIG. 84A shows ADRB1 expression in endothelial cells from different species. Resistant species are rat and mouse, sensitive species are cow and human.

FIG. 84B shows ADRB1 expression in human endothelial cells cultured in serum from different species. Resistant species are mouse and rat, sensitive species is human.

FIG. 85A shows ALDH1A2 expression in endothelial cells from different species. c

FIG. 85B shows ALDH1A2 expression in human endothelial cells cultured in serum from different species. Resistant species are mouse and rat, sensitive species is human.

FIG. 85C shows ALDH1A2 of human endothelial cells pretreated with HKEC (tolerant) and without pre-treatment prior to the indicated stimulations.

FIG. 86A shows ANKRD55 expression in endothelial cells from different species. Resistant species are rat and mouse, sensitive species are cow and human.

FIG. 86B shows ANKRD55 expression in human endothelial cells cultured in serum from different species. Resistant species are mouse and rat, sensitive species is human.

FIG. 87A shows CLU expression in endothelial cells from different species. Resistant species are rat and mouse, sensitive species are cow and humans.

FIG. 87B shows CLU expression in human endothelial cells cultured in serum from different species. Resistant species are mouse and rat, sensitive species is human.

FIG. 88A shows CTDSPL expression in endothelial cells from different species. Resistant species are rat and mouse, sensitive species are cow and humans.

FIG. 88B shows CTDSPL expression in human endothelial cells cultured in serum from different species.

Resistant species are mouse and rat, sensitive species is human.

FIG. 89A shows CTNNAL1 expression in endothelial cells from different species. Resistant species are mouse and rat, sensitive species is human.

FIG. 89B shows CTNNAL1 expression in human endothelial cells cultured in serum from different species. Resistant species are mouse and rat, sensitive species is human.

FIG. 90A shows DHFR expression in endothelial cells from different species. Resistant species are rat and mouse, sensitive species are cow and humans.

FIG. 90B shows DHFR expression in human endothelial cells cultured in serum from different species. Resistant species are mouse and rat, sensitive species is human.

FIG. 91A shows DNAJB4 expression in endothelial cells from different species. Resistant species are rat and mouse, sensitive species are cow and humans.

FIG. 91B shows DNAJB4 expression in endothelial cells cultured in serum from different species. Resistant species are mouse and rat, sensitive species is human.

FIG. 92A shows DPYD expression in endothelial cells from different species. Resistant species are rat and mouse, sensitive species are cow and humans.

FIG. 92B shows DPYD expression in human endothelial cells cultured in serum from different species. Resistant species are mouse and rat, sensitive species is human.

FIG. 93A shows FZD10 expression in human endothelial cells cultured in serum from different species. Resistant species are mouse and rat, sensitive species is human.

FIG. 93B shows FZD10 expression expression of human endothelial cells pretreated with HKEC (tolerant) and without pre-treatment prior to the indicated stimulations.

FIG. 94A shows GAB1 expression in endothelial cells from different species. Resistant species are rat and mouse, sensitive species are cow and humans.

FIG. 94B shows GAB1 expression in human endothelial cells cultured in serum from different species.

Resistant species are mouse and rat, sensitive species is human.

FIG. 95A shows GADD45G expression in endothelial cells from different species. Resistant species are rat and mouse, sensitive species are cow and humans.

FIG. 95B shows GADD45G expression in human endothelial cells cultured in serum from different species. Resistant species are mouse and rat, sensitive species is human.

FIG. 96A shows HCRTR1 expression in endothelial cells from different species. Resistant species are rat and mouse, sensitive species are cow and humans.

FIG. 96B shows HCRTR1 expression in human endothelial cells cultured in serum from different species. Resistant species are mouse and rat, sensitive species is human.

FIG. 97A shows IL7 expression in endothelial cells from different species. Resistant species are rat and mouse, sensitive species are cow and humans.

FIG. 97B shows IL7 expression in human endothelial cells cultured in serum from different species. Resistant species are mouse and rat, sensitive species is human.

FIG. 98A shows LRRC1 expression in endothelial cells from different species. Resistant species are rat and mouse, sensitive species are cow and humans.

FIG. 98B shows LRRC1 expression in human endothelial cells cultured in serum from different species.

Resistant species are mouse and rat, sensitive species is human.

FIG. 99A shows MYLIP expression in endothelial cells from different species. Resistant species are rat and mouse, sensitive species are cow and humans.

FIG. 99B shows MYLIP expression in human endothelial cells cultured in serum from different species. Resistant species are mouse and rat, sensitive species is human.

FIG. 100A shows NR1H3 expression in endothelial cells from different species. Resistant species are rat and mouse, sensitive species are cow and humans.

FIG. 100B shows NR1H3 expression in human endothelial cells cultured in serum from different species. Resistant species are mouse and rat, sensitive species is human.

FIG. 101A shows PCGF5 expression in endothelial cells from different species. Resistant species are rat and mouse, sensitive species are cow and humans.

FIG. 101B shows PCGF5 expression in human endothelial cells cultured in serum from different species. Resistant species are mouse and rat, sensitive species is human.

FIG. 102A shows PLSCR4 expression in endothelial cells from different species. Resistant species are rat and mouse, sensitive species are cow and humans.

FIG. 102B shows PLSCR4 expression in human endothelial cells cultured in serum from different species. Resistant species are mouse and rat, sensitive species is human.

FIG. 103A shows RAB11FIP1 expression in endothelial cell from different species. Resistant species are rat and mouse, sensitive species are cow and humans.

FIG. 103B shows RAB11FIP1 expression in human endothelial cells cultured in serum from different species. Resistant species are mouse and rat, sensitive species is human.

FIG. 104A shows RASD1 expression in endothelial cells from different species. Resistant species are rat and mouse, sensitive species are cow and humans.

FIG. 104B shows RASD1 expression in human endothelial cells cultured in serum from different species. Resistant species are mouse and rat, sensitive species is human.

FIG. 105A shows RGS16 expression in endothelial cells from different species. Resistant species are rat and mouse, sensitive species are cow and humans.

FIG. 105B shows RGS16 expression in human endothelial cells cultured in serum from different species. Resistant species are mouse and rat, sensitive species is human.

FIG. 106A shows RPGR expression in endothelial cells from different species. Resistant species are rat and mouse, sensitive species are cow and humans.

FIG. 106B shows RPGR expression in human endothelial cells cultured in serum from different species. Resistant species are mouse and rat, sensitive species is human.

FIG. 107A shows RUNX1T1 expression in endothelial cell from different species. Resistant species are rat and mouse, sensitive species are cow and humans.

FIG. 107B shows RUNX1T1 expression in human endothelial cells cultured in serum from different species. Resistant species are mouse and rat, sensitive species is human.

FIG. 108A shows SLC40A1 expression in endothelial cells from different species. Resistant species are rat and mouse, sensitive species are cow and humans.

FIG. 108B shows SLC40A1 expression in human endothelial cells cultured in serum from different species. Resistant species are mouse and rat, sensitive species is human.

FIG. 109A shows SLCO2A1 expression in endothelial cells from different species. Resistant species are rat and mouse, sensitive species are cow and humans.

FIG. 109B shows SLCO2A1 expression in human endothelial cells cultured in serum from different species. Resistant species are mouse and rat, sensitive species is human.

FIG. 110A shows STAT1 expression in endothelial cells from different species. Resistant species are rat and mouse, sensitive species are cow and humans.

FIG. 110B shows STAT1 expression in human endothelial cells cultured in serum from different species. Resistant species are mouse and rat, sensitive species is human.

FIG. 111A shows SULT1B1 expression in endothelial cells from different species. Resistant species are rat and mouse, sensitive species are cow and humans.

FIG. 111B shows SULT1B1 expression in human endothelial cells cultured in serum from different species. Resistant species are mouse and rat, sensitive species is human.

FIG. 112A shows TBC1D8 expression in endothelial cells from different species. Resistant species are rat and mouse, sensitive species are cow and humans.

FIG. 112B shows TBC1D8 expression in human endothelial cells cultured in serum from different species. Resistant species are mouse and rat, sensitive species is human.

FIG. 113A shows TGM2 expression in endothelial cells from different species. Resistant species are rat and mouse, sensitive species are cow and humans.

FIG. 113B shows TGM2 expression in human endothelial cells cultured in serum from different species. Resistant species are mouse and rat, sensitive species is human.

FIG. 114A shows WNT4 expression in endothelial cells cultured in serum from different species.

Resistant species are mouse and rat, sensitive species is human.

FIG. 114B shows WNT4 expression of human endothelial cells pretreated with HKEC (tolerant) and without pre-treatment prior to the indicated stimulations.

FIG. 115A shows FGF1 expression in endothelial cells from different species. Resistant species are rat and mouse, sensitive species are cow and humans.

FIG. 115B shows FGF1 expression of human endothelial cells pretreated with HKEC (tolerant) and without pre-treatment prior to the indicated stimulations.

### DETAILED DESCRIPTION OF THE INVENTION

The existence of some resistant species (for example, mice) that appear to be intrinsically tolerant to most inflammatory challenges compared to other species (for example, humans) provides the basis for the study described herein. We have reasoned that resistant species possess mechanism(s) to limit secondary inflammation following a pro-inflammatory stimulus and that these mechanisms are missing or ineffective in sensitive species (for example, humans).

Notably, animal species which are relatively resistant to inflammation do not appear to be necessarily relatively immunocompromised compared to species that are more sensitive to inflammatory challenge. For example, mice do not appear more immunocompromised than humans. This observation questions the current dogma that all secondarily induced inflammation is necessary for host protection and raises the possibility that the underlying mechanisms leading to the increased inflammatory resistance in these more resistant species may be exploited for human benefit.

This same variation in baseline sensitivity to inflammatory agonist challenge also raises important issues as to the validity of the use of many animal models that are currently used to reflect human disease. Tremendous resources are invested worldwide in the use of animal models for the development of new drugs and to explore the pathophysiology of inflammatory diseases. However, if the species that are studied have different innate tolerance to the same pro-inflammatory stimulus, the results from these models may be misinterpreted.

The mechanisms governing interspecies differences in natural tolerance are largely unknown. Most investigators have simply ignored this issue. Data comparing species response comes from animals that have been challenged with endotoxin. Because endotoxin has been injected into different animal species over the many decades, and because the biological portion of the endotoxin molecule—Lipid A—is similar in structure and potency for most endotoxins, it is possible to create a rough analysis of the sensitivity of different species to endotoxin (lipopolysaccharide, LPS). Species vary at least a million-fold in response to endotoxin challenge, with readouts being either death or severe disease. Notably, humans fall at the extreme sensitive end of the spectrum, whereas mice are at least 1000- to 100,000-fold more resistant.

For example, species that that are resistant to greater than 1 mg/kg LPS include mice, rats, rhesus monkeys (rhesus macaque) and baboon, and more sensitive species include rabbits, pigs, cows, sheep, chimps and humans.

Collectively, different vertebrate species vary over many orders of magnitude in their sensitivity to inflammatory stimuli, such as endotoxin (for example, LPS). Humans are extremely sensitive to LPS, whereas some species, including mice and many monkeys, are highly resistant to LPS. Accordingly, we have reasoned that agents conferring the type of resistance seen in mice and some non-human primates to humans are useful as effective therapies against diseases involving, for example, systemic inflammation.

As is disclosed herein, we studied and compared selected species that have high innate resistance (such as baboon, Rhesus monkey (rhesus macaque), rat, and mouse) to a pro-inflammatory challenge with selected species such as humans that have low innate resistance (high sensitivity to inflammatory challenge) in order to discover molecular gene and pathway targets that can be manipulated for therapeutic benefit.

Such therapeutic manipulation includes, for example, depending on the target and the treatment goal, the following:
1. Activation (e.g., induction) or down regulation (e.g., blockage) of specific genes and pathways and their downstream products to modify the inflammatory response in a species that is innately sensitive to inflammation (such as humans) in a way that would mimic a resistant species for the purpose of decreasing innate inflammation in disease settings in which this would be therapeutically desirable.
2. Conversely, such manipulation includes activation (e.g., induction) or down regulation (e.g., blockage) of specific genes and pathways and their downstream products to render the species even more sensitive than baseline when this may be therapeutically desirable (such as for example in the case of a vaccine adjuvant or cancer treatment in which this would be therapeutically beneficial).

### Overview

Described herein are methods for identifying gene targets that can be manipulated to develop therapies for modulating inflammation. Our methods for target identification take advantage of the observation as is described above that different species vary over many orders of magnitude in their sensitivity to inflammatory stimuli, such as endotoxin (LPS). Humans, for example, are sensitive to LPS, whereas many species used during preclinical development, such as mice and some non-human primates, are highly resistant to LPS. This sensitivity extends beyond LPS but to many conditions as is described herein.

We have compared, as is disclosed herein, the behavior of cells in whole blood and endothelial cells and constituents of the blood plasma in resistant species with that of sensitive species, or under conditions that alter sensitivity, to identify gene targets including their pathways, and proteins that regulate such differences.

Agents that confer the resistance seen in resistant species to humans are therefore considered useful as therapies against diseases involving systemic inflammation. Such exemplary diseases include sepsis, achalasia, acute or ischemic colitis, acute respiratory distress syndrome, allergy, allograft rejection, alveolitis, Alzheimer's disease, a neurological disease associated with amyloidosis, amebiasis, anaphylactic shock, angiitis, ankylosing spondylitis, appendicitis, arteritis, arthralgia, arthritides, asthma, atherosclerosis, Behcet's syndrome, Berger's disease, bronchiolitis, bronchitis, burns, cachexia, candidiasis, cerebral embolism, cerebral infarction, cholangitis, cholecystitis, chronic fatigue syndrome, celiac disease, congestive heart failure, Crohn's disease, cystic fibrosis, Dengue fever, dermatitis, dermatomyositis, disseminated bacteremia, diverticulitis, duodenal ulcers, emphysema, encephalitis, endocarditis, endotoxic shock, enteritis, eosinophilic granuloma, epididymitis, epiglottitis, fasciitis, filariasis, gastric ulcers, Goodpasture's syndrome, gout, graft-versus-host disease, granulomatosis, Guillan-Barre syndrome, hay fever, hepatitis, hepatitis B virus infection, hepatitis C virus infection, herpes infection, HIV infection, Hodgkin's disease, hydatid cysts, hyperpyrexia, immune complex disease, influenza, malaria, meningitis, multiple sclerosis, a multiple sclerosis-associated demyelination disease, myasthenia gravis, myocardial ischemia, myocarditis, neuralgia, neuritis, organ ischemia, organ necrosis, osteomyelitis, Paget's disease, pancreatitis, ulcerative pancreatitis, pseudomembranous colitis, pancreatitis, paralysis, peptic ulcers, periarteritis nodosa, pericarditis, periodontal disease, peritonitis, pharyngitis, pleurisy, pneumonitis, pneumonoultramicroscopicsilicovolcanokoniosis, prostatitis, pseudomembranous Reiter's syndrome, reperfusion injury, respiratory syncytial virus infection, rheumatic fever, rheumatoid arthritis, rhinitis, sarcoidosis, septic abortion, septicemia, sinusitis, spinal cord injury, sunburn, synovitis, systemic lupus erythematosus, systemic lupus erythrocytosis, thrombophlebitis, thyroiditis, Type I diabetes, ulcerative colitis, urethritis, urticaria, uveitis, vaginitis, vasculitis, warts, wheals, or Whipple's disease. Still other conditions include chronic inflammation caused by, for example, bacterial, viral, and parasitic infections, or chemical irritants (e.g., asbestos).

Conversely, agents that further increase the sensitivity of humans are accordingly considered as useful therapies in diseases in which there is insufficient innate immunity, such as in most cancer. Exemplary cancers include melanoma and carcinomas of the head and neck, breast, lung, prostate, bladder, kidney, colon, ovary, and esophagus.

Diseases Associated with Inflammation

Many diseases have inflammation as either a primary or secondary driver of illness. Such diseases include infection from any cause with secondary induced inflammation, acute or chronic autoimmune diseases including inflammatory bowel disease, rheumatoid arthritis, and vasculitis as well as other chronic diseases such as Alzheimer's disease and atherosclerosis or other diseases in which chronic inflammation is present.

The fact that some species are intrinsically resistant to innate inflammatory stimuli but are not immunocompromised, whereas others, including humans, are intrinsically highly sensitive to inflammatory stimuli, indicates that innate inflammation is not necessary for protection against microorganisms, and therefore that therapies that are based upon mimicking species differences to alter inflammation would not necessarily result in alterations in immunity. Accordingly, inflammation can be dissociated from immunity.

The response of whole blood, for example, to stimulation with endotoxin or killed bacteria, or more specifically the secondary induction of cytokines such as but not limited to TNF or IL6 is thought to mimic the in vivo condition. For example, whole blood from sensitive species such as humans stimulated with LPS or killed E. coli produces large concentrations of TNF, whereas there is almost no TNF produced under identical conditions in the resistant mouse species whole blood. This is consistent with the relative difference in sensitivity to LPS in the two species in vivo. We have, as is described below, used a whole blood system to assess the state of the blood cells in response to LPS in different species.

Endothelial cells provide a highly dynamic interactive lining of blood vessels. These cells respond to stimulation with pro-inflammatory stimuli to mediate inflammation, while at the same time creating a barrier to prevent leakage of cells and plasma into the surrounding tissues. Loss of the endothelial barrier leads to decreased oxygenation and increased inflammation. In addition, red blood cells (RBCs) are released into areas of injury, where their breakdown products of hemoglobin and heme likely further amplify inflammation. This leakage across the endothelial barrier may be particularly important in lungs where fluid in alveoli may interfere with air exchange and lead to hypoxia.

### Methodology

1. Below we describe methods to identify therapeutic targets for treatment of diseases involving dysregulated innate immunity. Specifically, drug targets were discovered by comparing whole blood cell transcriptomics and plasma proteomics from six (6) species that we classified as having sensitive innate immunity and four (4) species that we classified as resistant innate immunity (based upon published data for endotoxin challenge). We also developed drug targets for endothelial cells by identifying shared transcriptomic features when comparing gene expression between endothelial cells derived from different species, human endothelial cells exposed to the serum of either sensitive or resistant species, or human endothelial cells rendered resistant to LPS stimulation by prior treatment (tolerance).
2. Targets described herein are listed, for example, either as genes or serum proteins.
   a. In the case of genes, therapeutics will affect or mimic, indirectly or directly, the gene's expression or the gene product's activity. Useful therapeutics include agents or methods which manipulate gene expression, including but not limited to siRNA or directed CRISPR or similar gene editing methods, or injection of host cells in which the gene of interest or its expression has been specifically altered. Cells can also be altered either in vivo or ex vivo after which the altered cell is injected into the host. The gene product, if desired, may also be targeted by injecting polyclonal or monoclonal antibodies which are engineered to alter the gene product of the targeted genes, or by large or small molecules that directly or indirectly alter the behavior of the gene product.
   b. In the case of serum proteins, therapeutics may be peptides or recombinant proteins or antibodies that mimic or enhance their biological activity, or agents that prevent functional binding of the protein to its cellular receptor or otherwise interfere with their function.
3. Targets can also be separated by the analysis method used to identify them, for example, leukocyte transcriptomics, endothelial cell transcriptomics, and serum proteomics. Such separation provides a basis for predicting their site of functional importance.
4. The studies described herein include data using several existing drugs or therapeutic candidates. These may or may not be approved therapies for treatment of indications described herein. We accordingly envisage repurposing these drugs for new indications, with or without modification, or designing new drugs with the same molecular targets.
5. An effective therapeutic may either inhibit or promote a target's activity in order to achieve the desired effect.
6. Some targets described herein are known mediators of the immune response. Such targets provide a basis for validating the usefulness of the disclosed methods, and accordingly provide support for targets for which we do not otherwise have supporting data.
7. Exemplary targets are disclosed throughout the specification. Preferred targets are listed in Tables 6 and 7. Other targets (including known isoforms) may have identity (for example 40 percent identity) with a target found in these tables. Nucleotide sequences of the targets are readily obtained according to standard techniques. As used herein the term “percent identity” refers to percent (%) sequence identity with respect to a reference polynucleotide or polypeptide sequence following alignment by standard techniques. Alignment for purposes of determining percent nucleic acid or amino acid sequence identity can be achieved in various ways that are within the capabilities of one of skill in the art, for example, using publicly available computer software such as BLAST, BLAST-2, PSI-BLAST, or Megalign software. Those skilled in the art can determine appropriate parameters for aligning sequences, including any algorithms needed to achieve maximal alignment over the full length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:

100 multiplied by (the fraction X/Y)
where X is the number of nucleotides or amino acids scored as identical matches by a sequence alignment program (e.g., BLAST) in that program's alignment of A and B, and where Y is the total number of nucleotides or amino acids in B. In some embodiments, sequence identity, for example, in homologues of proteins (as noted in Tables 6 and 7) will have at least about 40%, 50%, 60%, 70%, 80%, 85%, 90%, or even 95% or greater amino acid or nucleic acid sequence identity, alternatively at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% or greater amino acid sequence or nucleic acid identity, to a native sequence as disclosed herein.

### Identifying Therapeutics

As discussed herein, our experimental results identified targets such as genes and gene products associated with sensitive or resistant responses. Identifying agents that modulate such targets is accomplished using screening methodologies such as those described herein. In general, screening methods provide a straightforward means for selecting natural product extracts or compounds of interest from a large population which are further evaluated and condensed to a few active and selective materials. Constituents of this pool are then purified and evaluated for their therapeutic effects.

### Screening

As discussed herein, our experimental results identified targets such as genes and gene products associated with sensitive or resistant responses. Identifying agents that modulate such targets is accomplished using screening methodologies such as those described herein.

In general, screening methods provide a straightforward means for selecting natural product extracts or compounds of interest from a large population which are further evaluated and condensed to a few active and selective materials. Constituents of this pool are then purified and evaluated for their therapeutic effects.

### Compounds

In general, drugs are identified from libraries of both known compounds, natural product or synthetic (or semi-synthetic) extracts or chemical libraries according to methods known in the art. The screening method of the present invention is appropriate and useful for testing compounds from a variety of sources. The initial screens may be performed using a diverse library of compounds, but the method is suitable for a variety of other compounds and compound libraries. Such compound libraries can be combinatorial libraries, natural product libraries, or other small molecule libraries. In addition, compounds from commercial sources can be tested, as well as commercially available analogs of identified inhibitors.

For example, those skilled in the field of drug discovery and development will understand that the precise source of test extracts or compounds is not critical to the screening procedure(s) of the invention. Accordingly, virtually any number of chemical extracts or compounds can be screened using the methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds.

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their activity should be employed whenever possible.

Since many of the compounds in libraries such as combinatorial and natural products libraries, as well as in natural products preparations, are not characterized, the screening methods of this invention provide novel compounds which are active as inhibitors or inducers in the particular screens, in addition to identifying known compounds which are active in the screens. Therefore, this invention includes such novel compounds, as well as the use of both novel and known compounds in pharmaceutical compositions and methods of treating.

There now follows a description a variety of therapeutics useful treating virtually any number of conditions described herein. Such therapeutics are illustrative and are not to be construed as limiting.

### Therapeutics

Antisense

Antisense nucleic acids typically suppress gene expression by inducing an RNAse-H-mediated degradation of a target nucleic acid or by sterically blocking the target sequence thereby eliminating reliance on steric blockage of a target sequence. Antisense nucleic acids capable of inducing an RNAse-H-mediated degradation of the target nucleic acid are typically polynucleotides. Antisense nucleic acids capable of sterically blocking a target sequence may be polynucleotides, PNAs, or morpholino nucleic acids. Approaches to designing antisense oligonucleotides are known in the art, see, e.g., US 2015/0031747.

In certain embodiments, an antisense nucleic acid has a nucleobase sequence that, when written in the 5′ to 3′ direction, comprises the reverse complement of the target segment of a target nucleic acid to which it is targeted.

In certain embodiments, the antisense nucleic acid has a total of 12 to 30 subunits in length. In other words, such antisense nucleic acids are from 12 to 30 linked subunits. In other embodiments, the antisense nucleic acid is 8 to 80, 12 to 50, 15 to 30, 18 to 24, 19 to 22, or 20 linked subunits. In certain such embodiments, the antisense nucleic acids are 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 linked subunits in length, or a range defined by any two of the above values. In some embodiments the antisense nucleic acid is an antisense nucleic acid, and the linked subunits are nucleosides.

In certain embodiments, antisense nucleic acids targeted to a target nucleic acid may be shortened or truncated. For example, a single subunit may be deleted from the 5′ end (5′ truncation), or alternatively from the 3′ end (3′ truncation). A shortened or truncated antisense nucleic acid targeted to a target nucleic acid may have two subunits deleted from the 5′ end, or alternatively may have two subunits deleted from the 3′ end, of the antisense nucleic acid. Alternatively, the deleted nucleosides may be dispersed throughout the antisense nucleic acid, for example, in an antisense nucleic acid having one nucleoside deleted from the 5′ end and one nucleoside deleted from the 3′ end.

When a single additional subunit is present in a lengthened antisense nucleic acid, the additional subunit may be located at the 5′ or 3′ end of the antisense nucleic acid. When two or more additional subunits are present, the added subunits may be adjacent to each other; for example, in an antisense nucleic acid having two subunits added to the 5′ end (5′ addition), or alternatively to the 3′ end (3′ addition), of the antisense nucleic acid. Alternatively, the added subunits may be dispersed throughout the antisense nucleic acid, for example, in an antisense nucleic acid having one subunit added to the 5′ end and one subunit added to the 3′ end.

It is possible to increase or decrease the length of an antisense nucleic acid, such as an antisense nucleic acid, and/or introduce mismatch bases without eliminating activity. For example, in Woolf et al. (Proc. Natl. Acad. Sci. USA 89:7305-7309, 1992), a series of antisense nucleic acids 13-25 nucleobases in length were tested for their ability to induce cleavage of a target RNA in an oocyte injection model. Antisense nucleic acids 25 nucleobases in length with 8 or 11 mismatch bases near the ends of the antisense nucleic acids were able to direct specific cleavage of the target mRNA, albeit to a lesser extent than the antisense nucleic acids that contained no mismatches. Similarly, target specific cleavage was achieved using 13 nucleobase antisense nucleic acids, including those with 1 or 3 mismatches.

Gautschi et al. (J. Natl. Cancer Inst. 93:463-471, March 2001) demonstrated the ability of an oligonucleotide having 100% complementarity to the bcl-2 mRNA and having 3 mismatches to the bcl-xL mRNA to reduce the expression of both bcl-2 and bcl-xL in vitro and in vivo. Furthermore, this oligonucleotide demonstrated potent anti-tumor activity in vivo.

Maher and Dolnick (Nuc. Acid. Res. 16:3341-3358, 1988) tested a series of tandem 14 nucleobase antisense nucleic acids, and 28 and 42 nucleobase antisense nucleic acids comprised of the sequence of two or three of the tandem antisense nucleic acids, respectively, for their ability to arrest translation of human DHFR in a rabbit reticulocyte assay. Each of the three 14 nucleobase antisense nucleic acids alone was able to inhibit translation, albeit at a more modest level than the 28 or 42 nucleobase antisense nucleic acids.

In certain embodiments, antisense nucleic acids targeted to a target nucleic acid have chemically modified subunits arranged in patterns, or motifs, to confer to the antisense nucleic acids properties, such as enhanced inhibitory activity, increased binding affinity for a target nucleic acid, or resistance to degradation by in vivo nucleases.

Chimeric antisense nucleic acids typically contain at least one region modified so as to confer increased resistance to nuclease degradation, increased cellular uptake, increased binding affinity for the target nucleic acid, and/or increased inhibitory activity. A second region of a chimeric antisense nucleic acid may optionally serve as a substrate for the cellular endonuclease RNase H, which cleaves the RNA strand of an RNA:DNA duplex.

Antisense nucleic acids having a gapmer motif are considered chimeric antisense nucleic acids. In a gapmer, an internal region having a plurality of nucleotides that supports RNaseH cleavage is positioned between external regions having a plurality of nucleotides that are chemically distinct from the nucleosides of the internal region. In the case of an antisense nucleic acid having a gapmer motif, the gap segment generally serves as a substrate for endonuclease cleavage, while the wing segments comprise modified nucleosides. In certain embodiments, the regions of a gapmer are differentiated by the types of sugar moieties comprising each distinct region. The types of sugar moieties that are used to differentiate the regions of a gapmer may, in some embodiments, include β-D-ribonucleosides, β-D-deoxyribonucleosides, 2′-modified nucleosides (such 2′-modified nucleosides may include 2′-MOE, and 2′-O—CH₃, among others), and bicyclic sugar modified nucleosides (such bicyclic sugar modified nucleosides may include those having a 4′-(CH2)n-O-2′ bridge, where n=1 or n=2). Preferably, each distinct region comprises uniform sugar moieties. The wing-gap-wing motif is frequently described as “X—Y—Z”, where “X” represents the length of the 5′ wing region, “Y” represents the length of the gap region, and “Z” represents the length of the 3′ wing region. As used herein, a gapmer described as “X—Y—Z” has a configuration such that the gap segment is positioned immediately adjacent each of the 5′ wing segment and the 3′ wing segment. Thus, no intervening nucleotides exist between the 5′ wing segment and gap segment, or the gap segment and the 3′ wing segment. Any of the antisense nucleic acids described herein can have a gapmer motif. In some embodiments, X and Z are the same, in other embodiments they are different. In a preferred embodiment, Y is between 8 and 15 nucleotides. X, Y or Z can be any of 1, 2, 3, 4, 5, 6, 7, 8, 9, 10, 11, 12, 13, 14, 15, 16, 17, 18, 19, 20, 25, 30, or more nucleotides. Thus, gapmers described herein include, but are not limited to, for example, 5-10-5, 4-8-4, 4-12-3, 4-12-4, 3-14-3, 2-13-5, 2-16-2, 1-18-1, 3-10-3, 2-10-2, 1-10-1 or 2-8-2.

In certain embodiments, the antisense nucleic acid has a “wingmer” motif, having a wing-gap or gap-wing configuration, i.e. an X-Y or Y—Z configuration, as described above, for the gapmer configuration. Thus, wingmer configurations described herein include, but are not limited to, for example, 5-10, 8-4, 4-12, 12-4, 3-14, 16-2, 18-1, 10-3, 2-10, 1-10, 8-2, 2-13, or 5-13.

In certain embodiments, antisense nucleic acids targeted to a target nucleic acid possess a 5-10-5 gapmer motif.

In certain embodiments, antisense nucleic acids targeted to a target nucleic acid possess a 3-14-3 gapmer motif.

In certain embodiments, antisense nucleic acids targeted to a target nucleic acid possess a 2-13-5 gapmer motif.

In certain embodiments, antisense nucleic acids targeted to a target nucleic acid possess a 2-12-2 gapmer motif.

In certain embodiments, an antisense nucleic acid targeted to a target nucleic acid has a gap-widened motif.

In certain embodiments, a gap-widened antisense nucleic acid targeted to a target nucleic acid has a gap segment of fourteen 2′-deoxyribonucleotides positioned immediately adjacent to and between wing segments of three chemically modified nucleosides. In certain embodiments, the chemical modification comprises a 2′-sugar modification. In another embodiment, the chemical modification comprises a 2′-MOE sugar modification.

In certain embodiments, a gap-widened antisense nucleic acid targeted to a target nucleic acid has a gap segment of thirteen 2′-deoxyribonucleotides positioned immediately adjacent to and between a 5′ wing segment of two chemically modified nucleosides and a 3′ wing segment of five chemically modified nucleosides. In certain embodiments, the chemical modification comprises a 2′-sugar modification. In another embodiment, the chemical modification comprises a 2′-MOE sugar modification.

In certain embodiments, such oligonucleotides have a gap segment of 9, 10, or more linked deoxynucleosides. In certain embodiments, such gap segment is between two wing segments that independently have 1, 2, 3, 4, or 5 linked modified nucleosides. In certain embodiments, one or more modified nucleosides in the wing segment have a modified sugar. In certain embodiments, the modified sugar is a bicyclic sugar. In certain embodiments, the modified nucleoside is an LNA nucleoside. In certain embodiments, the modified nucleoside is a 2′-substituted nucleoside. In certain embodiments, 2′ substituted nucleosides include nucleosides with bicyclic sugar modifications. In certain embodiments, the modified nucleoside is a 2′-MOE nucleoside. In certain embodiments, the modified nucleoside is a constrained ethyl (cEt) nucleoside. In certain embodiments, the modified nucleoside is a F-HNA nucleoside. In certain embodiments, each modified nucleoside in each wing segment is independently a 2′-MOE nucleoside or a nucleoside with a bicyclic sugar modification such as a constrained ethyl (cEt) nucleoside or LNA nucleoside. In certain embodiments, each modified nucleoside in each wing segment is independently a 2′-MOE nucleoside, a nucleoside with a bicyclic sugar modification such as a constrained ethyl (cEt) nucleoside or LNA nucleoside, or a 2′-deoxyribonucleoside.

In certain embodiments, the compounds or compositions comprise a modified oligonucleotide consisting of 10 to 30 linked nucleosides and having a nucleobase sequence comprising at least 8, 9, 10, 11, 12, 13, 14, 15, 16, 17, 18, 19, or 20 contiguous nucleobases of any of nucleotides encoding the polypeptides described in Tables 6 ad 7. In certain embodiments, such oligonucleotides have a gap segment of 8, 9, 10, or more linked deoxynucleosides. In certain embodiments, such gap segment is between two wing segments that independently have 1, 2, 3, 4, 5, 6, 7, or 8 linked modified nucleosides. In certain embodiments, one or more modified nucleosides in the wing segment have a modified sugar. In certain embodiments, the modified sugar is a bicyclic sugar. In certain embodiments, the modified nucleoside is an LNA nucleoside. In certain embodiments, the modified nucleoside is a 2′-substituted nucleoside. In certain embodiments, 2′ substituted nucleosides include nucleosides with bicyclic sugar modifications. In certain embodiments, the modified nucleoside is a 2′-MOE nucleoside. In certain embodiments, the modified nucleoside is a constrained ethyl (cEt) nucleoside. In certain embodiments, the modified nucleoside is a F-HNA nucleoside. In certain embodiments, each modified nucleoside in each wing segment is independently a 2′-MOE nucleoside, a nucleoside with a bicyclic sugar modification such as a constrained ethyl (cEt) nucleoside or LNA nucleoside, or a 2′-deoxyribonucleoside.

In certain embodiments, the modified oligonucleotide is 16 nucleosides in length and has a gap segment of 9 linked nucleosides. In certain embodiments, the modified oligonucleotide is 16 nucleosides in length and has a gap segment of 10 linked nucleosides. In certain embodiments, the modified oligonucleotide is 20 nucleosides in length and has a gap segment of 10 linked nucleosides. In certain embodiments, the modified oligonucleotide has a wing segment on the 5′ end and 3′ end of the gap each independently having 1, 2, 3, 4, or 5 sugar modified nucleosides. In certain embodiments, each sugar modified nucleoside is independently a 2′-MOE nucleoside, a nucleoside with a bicyclic sugar moiety such as a constrained ethyl (cEt) nucleoside or LNA nucleoside, or a F-HNA nucleoside. In certain embodiments, each modified nucleoside in each wing segment is independently a 2′-MOE nucleoside, a nucleoside with a bicyclic sugar modification such as a constrained ethyl (cEt) nucleoside or LNA nucleoside, a 2′-deoxyribonucleoside, or a F-HNA nucleoside.

In certain embodiments, the compounds or compositions comprise a salt of the modified oligonucleotide.

In certain embodiments, the modified oligonucleotide comprises: a) a gap segment consisting of linked deoxynucleosides; b) a 5′ wing segment consisting of linked nucleosides; and c) a 3′ wing segment consisting of linked nucleosides. The gap segment is positioned between the 5′ wing segment and the 3′ wing segment and each nucleoside of each wing segment comprises a modified sugar.

In certain embodiments, the modified oligonucleotide consists of 16 linked nucleosides, the gap segment consisting of 10 linked deoxynucleosides, the 5′ wing segment consisting of three linked nucleosides, the 3′ wing segment consisting of three linked nucleosides, each nucleoside of each wing segment comprises a 2′-O-methoxyethyl sugar and/or a constrained ethyl (cEt) sugar, each internucleoside linkage is a phosphorothioate linkage and each cytosine is a 5-methylcytosine. In some aspects, each of the three linked nucleosides of the 5′ wing segment is a 2′-O-methoxyethyl nucleoside and each of the three linked nucleosides of the 3′ wing segment is a constrained ethyl (cEt) nucleoside. In other aspects, the three linked nucleosides of the 5′ wing segment are a 2′-O-methoxyethyl nucleoside, a constrained ethyl (cEt) nucleoside, and a constrained ethyl (cEt) nucleoside in the 5′ to 3′ direction, and the three linked nucleosides of the 3′ wing segment are a constrained ethyl (cEt) nucleoside, a constrained ethyl (cEt) nucleoside, and a 2′-O-methoxyethyl nucleoside in the 5′ to 3′ direction. In other aspects, the three linked nucleosides of the 5′ wing segment are a 2′-O-methoxyethyl nucleoside, 2′-O-methoxyethyl nucleoside, and a constrained ethyl (cEt) nucleoside in the 5′ to 3′ direction, and the three linked nucleosides of the 3′ wing segment are a constrained ethyl (cEt) nucleoside, a constrained ethyl (cEt) nucleoside, and a 2′-O-methoxyethyl nucleoside in the 5′ to 3′ direction.

In certain embodiments, the modified oligonucleotide consists of 16 linked nucleosides, the gap segment consisting of 10 linked deoxynucleosides, the 5′ wing segment consisting of one nucleoside, the 3′ wing segment consisting of five linked nucleosides, each nucleoside of each wing segment comprises a 2′-O-methoxyethyl sugar and/or a constrained ethyl (cEt) sugar, each internucleoside linkage is a phosphorothioate linkage and each cytosine is a 5-methylcytosine. In some aspects, the nucleoside of the 5′ wing segment is a constrained ethyl (cEt) nucleoside and the five linked nucleosides of the 3′ wing segment are a constrained ethyl (cEt) nucleoside, 2′-O-methoxyethyl nucleoside, a constrained ethyl (cEt) nucleoside, a 2′-O-methoxyethyl nucleoside, and a 2′-O-methoxyethyl nucleoside in the 5′ to 3′ direction.

In certain embodiments, the modified oligonucleotide consists of 16 linked nucleosides, the gap segment consisting of 9 linked deoxynucleosides, the 5′ wing segment consisting of five linked nucleosides, the 3′ wing segment consisting of two linked nucleosides, each nucleoside of each wing segment comprises a 2′-O-methoxyethyl sugar, a 2′-deoxyribose, and/or a constrained ethyl (cEt) sugar, each internucleoside linkage is a phosphorothioate linkage and each cytosine is a 5-methylcytosine. In some aspects, the five linked nucleosides of the 5′ wing segment are a constrained ethyl (cEt) nucleoside, a 2′-deoxynucleoside, a constrained ethyl (cEt) nucleoside, a 2′-deoxynucleoside, and a constrained ethyl (cEt) sugar and the two linked nucleosides of the 3′ wing segment are a 2′-O-methoxyethyl nucleoside and a 2′-O-methoxyethyl sugar in the 5′ to 3′ direction.

In some embodiments, hybridization occurs between an antisense nucleic acid disclosed herein and a target nucleic acid. The most common mechanism of hybridization involves hydrogen bonding (e.g., Watson-Crick, Hoogsteen or reversed Hoogsteen hydrogen bonding) between complementary nucleobases of the nucleic acid molecules.

Hybridization can occur under varying conditions. Stringent conditions are sequence-dependent and are determined by the nature and composition of the nucleic acid molecules to be hybridized.

Methods of determining whether a sequence is specifically hybridizable to a target nucleic acid are well known in the art. In certain embodiments, the antisense nucleic acids provided herein are specifically hybridizable with a target nucleic acid.

An antisense nucleic acid and a target nucleic acid are complementary to each other when a sufficient number of nucleobases of the antisense nucleic acid can hydrogen bond with the corresponding nucleobases of the target nucleic acid, such that a desired effect will occur (e.g., antisense inhibition of a target nucleic acid, such as a target nucleic acid).

Noncomplementary nucleobases between an antisense nucleic acid and a target nucleic acid may be tolerated provided that the antisense nucleic acid remains able to specifically hybridize to a target nucleic acid. Moreover, an antisense nucleic acid may hybridize over one or more segments of a target nucleic acid such that intervening or adjacent segments are not involved in the hybridization event (e.g., a loop structure, mismatch or hairpin structure).

In certain embodiments, the antisense nucleic acids provided herein, or a specified portion thereof, are, or are at least, 70%, 80%, 85%, 86%, 87%, 88%, 89%, 90%, 91%, 92%, 93%, 94%, 95%, 96%, 97%, 98%, 99%, or 100% complementary to a target nucleic acid, a target region, target segment, or specified portion thereof. Percent complementarity of an antisense nucleic acid with a target nucleic acid can be determined using routine methods.

For example, an antisense nucleic acid in which 18 of 20 nucleobases of the antisense nucleic acid are complementary to a target region, and would therefore specifically hybridize, would represent 90 percent complementarity. In this example, the remaining noncomplementary nucleobases may be clustered or interspersed with complementary nucleobases and need not be contiguous to each other or to complementary nucleobases. As such, an antisense nucleic acid which is 18 nucleobases in length having 4 (four) noncomplementary nucleobases which are flanked by two regions of complete complementarity with the target nucleic acid would have 77.8% overall complementarity with the target nucleic acid and would thus fall within the scope of the present invention. Percent complementarity of an antisense nucleic acid with a region of a target nucleic acid can be determined routinely using BLAST programs (basic local alignment search tools) and PowerBLAST programs known in the art (Altschul et al., J. Mol. Biol., 1990, 215, 403 410; Zhang and Madden, Genome Res., 1997, 7, 649 656). Percent homology, sequence identity or complementarity can be determined by, for example, the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482 489).

In certain embodiments, the antisense nucleic acids provided herein, or specified portions thereof, are fully complementary (i.e. 100% complementary) to a target nucleic acid, or specified portion thereof. For example, an antisense nucleic acid may be fully complementary to a target nucleic acid, or a target region, or a target segment or target sequence thereof. As used herein, “fully complementary” means each nucleobase of an antisense nucleic acid is capable of precise base pairing with the corresponding nucleobases of a target nucleic acid. For example, a 20 nucleobase antisense nucleic acid is fully complementary to a target sequence that is 400 nucleobases long, so long as there is a corresponding 20 nucleobase portion of the target nucleic acid that is fully complementary to the antisense nucleic acid. Fully complementary can also be used in reference to a specified portion of the first and/or the second nucleic acid. For example, a 20 nucleobase portion of a 30 nucleobase antisense nucleic acid can be “fully complementary” to a target sequence that is 400 nucleobases long. The 20 nucleobase portion of the 30 nucleobase oligonucleotide is fully complementary to the target sequence if the target sequence has a corresponding 20 nucleobase portion wherein each nucleobase is complementary to the 20 nucleobase portion of the antisense nucleic acid. At the same time, the entire 30 nucleobase antisense nucleic acid may or may not be fully complementary to the target sequence, depending on whether the remaining 10 nucleobases of the antisense nucleic acid are also complementary to the target sequence.

The location of a non-complementary nucleobase may be at the 5′ end or 3′ end of the antisense nucleic acid. Alternatively, the non-complementary nucleobase or nucleobases may be at an internal position of the antisense nucleic acid. When two or more non-complementary nucleobases are present, they may be contiguous (i.e. linked) or non-contiguous. In one embodiment, a non-complementary nucleobase is located in the wing segment of a gapmer antisense nucleic acid.

In certain embodiments, antisense nucleic acids that are, or are up to 12, 13, 14, 15, 16, 17, 18, 19, or nucleobases in length comprise no more than 4, no more than 3, no more than 2, or no more than 1 non-complementary nucleobase(s) relative to a target nucleic acid, such as a target nucleic acid, or specified portion thereof.

In certain embodiments, antisense nucleic acids that are, or are up to 12, 13, 14, 15, 16, 17, 18, 19, 20, 21, 22, 23, 24, 25, 26, 27, 28, 29, or 30 nucleobases in length comprise no more than 6, no more than 5, no more than 4, no more than 3, no more than 2, or no more than 1 non-complementary nucleobase(s) relative to a target nucleic acid, such as a target nucleic acid, or specified portion thereof.

The antisense nucleic acids provided herein also include those which are complementary to a portion of a target nucleic acid. As used herein, “portion” refers to a defined number of contiguous (i.e. linked) nucleobases within a region or segment of a target nucleic acid. A “portion” can also refer to a defined number of contiguous nucleobases of an antisense nucleic acid. In certain embodiments, the antisense nucleic acids are complementary to at least an 8 nucleobase portion of a target segment. In certain embodiments, the antisense nucleic acids are complementary to at least a 12 nucleobase portion of a target segment. In certain embodiments, the antisense nucleic acids are complementary to at least a 15 nucleobase portion of a target segment. Also contemplated are antisense nucleic acids that are complementary to at least a 9, 10, 11, 12, 13, 14, 15, 16, 17, 18, 19, 20, or more nucleobase portion of a target segment, or a range defined by any two of these values.

The antisense nucleic acids provided herein may also have a defined percent identity to a particular target or a portion thereof. As used herein, an antisense nucleic acid is identical to the sequence disclosed herein if it has the same nucleobase pairing ability. For example, a RNA which contains uracil in place of thymidine in a disclosed DNA sequence would be considered identical to the DNA sequence since both uracil and thymidine pair with adenine. Shortened and lengthened versions of the antisense nucleic acids described herein as well as compounds having non-identical bases relative to the antisense nucleic acids provided herein also are contemplated. The non-identical bases may be adjacent to each other or dispersed throughout the antisense nucleic acid. Percent identity of an antisense nucleic acid is calculated according to the number of bases that have identical base pairing relative to the sequence to which it is being compared.

In certain embodiments, the antisense nucleic acids, or portions thereof, are at least 70%, 75%, 80%, 85%, 90%, 95%, 96%, 97%, 98%, 99% or 100% identical to one or more antisense nucleic acids designed in view of the genes expressing one or more of the polypeptides described in Table 6 or 7, or a portion thereof.

In certain embodiments, a portion of the antisense nucleic acid is compared to an equal length portion of the target nucleic acid. In certain embodiments, an 8, 9, 10, 11, 12, 13, 14, 15, 16, 17, 18, 19, 20, 21, 22, 23, 24, or 25 nucleobase portion is compared to an equal length portion of the target nucleic acid.

In certain embodiments, a portion of the antisense nucleic acid is compared to an equal length portion of the target nucleic acid. In certain embodiments, an 8, 9, 10, 11, 12, 13, 14, 15, 16, 17, 18, 19, 20, 21, 22, 23, 24, or 25 nucleobase portion is compared to an equal length portion of the target nucleic acid.

A nucleoside is a base-sugar combination. The nucleobase (also known as base) portion of the nucleoside is normally a heterocyclic base moiety. Nucleotides are nucleosides that further include a phosphate group covalently linked to the sugar portion of the nucleoside. For those nucleosides that include a pentofuranosyl sugar, the phosphate group can be linked to the 2′, 3′ or 5′ hydroxyl moiety of the sugar. Oligonucleotides are formed through the covalent linkage of adjacent nucleosides to one another, to form a linear polymeric oligonucleotide. Within the oligonucleotide structure, the phosphate groups are commonly referred to as forming the internucleoside linkages of the oligonucleotide.

Modifications to antisense nucleic acids encompass substitutions or changes to internucleoside linkages, sugar moieties, or nucleobases. Modified antisense nucleic acids are often preferred over native forms because of desirable properties such as, for example, enhanced cellular uptake, enhanced affinity for nucleic acid target, increased stability in the presence of nucleases, or increased inhibitory activity.

Chemically modified nucleosides may also be employed to increase the binding affinity of a shortened or truncated antisense nucleic acid for its target nucleic acid. Consequently, comparable results can often be obtained with shorter antisense nucleic acids that have such chemically modified nucleosides.

siRNA

siRNA nucleic acids are nucleic acids that predominantly rely on RISC-mediated transcript degradation. Typically, siRNA is a hybridized polynucleotide having a passenger strand and a guide strand, each of the strands having 16-26 contiguous nucleotides. An siRNA may include one or two 3′ overhangs (e.g., 2-nucleotide-long overhangs), or an siRNA may have one blunt end (where the 3′-end of the guide strand is hybridized to the 5′-end of the passenger strand). Approaches to desigining siRNAs are known in the art, see, e.g., U.S. Pat. No. 9,234,196. siRNA design tools are available for siRNA sequence selection, e.g., the design tools available from Integrated DNA Technologies, Inc. (idtdna.com), DHARMACON (dharmacon.horizondiscovery.com), or ThermoFisher Scientific (rnaidesigner.thermofisher.com).

The siRNAs of the compositions featured herein include an RNA strand (the antisense strand) having a region which is less than 30 nucleotides in length, generally 19-24 nucleotides in length, and is substantially complementary to at least part of a target mRNA transcript. The use of these siRNAs provides for the targeted degradation of mRNAs of target genes. Low dosages of siRNAs in particular can specifically and efficiently mediate RNAi, resulting in inhibition of expression of a target gene. Methods and compositions including siRNAs may be useful for diseases described herein.

The methods and compositions containing an siRNA may be useful for treating pathological processes mediated by the target gene expression. In an embodiment, method described herein may include administering to a mammal in need thereof a therapeutically effective amount of an siRNA targeted to the target gene. In an embodiment, an siRNA is administered to the mammal at 0.01 to 25 mg/kg (e.g., about 0.01, 0.1, 0.5, 1.0, 2, 2.5, 3, 4, 5, 6, 7, 8, 9, 10, 11, 12, 13, 14, 15, 16, 17, 18, 19, 20, 21, 22, 23, 24, or 25 mg/kg).

The following detailed description discloses how to make and use the compositions containing siRNAs to inhibit the expression of a target gene, as well as compositions and methods for treating diseases and disorders caused by the expression of this gene. The compositions described herein may include an siRNA having an antisense strand comprising a region of complementarity which is less than 30 nucleotides in length, generally 19-24 nucleotides in length, and is substantially complementary to at least part of an RNA transcript of a target gene, together with a pharmaceutically acceptable carrier. The siRNA may have an antisense strand having a region of complementarity which is less than 30 nucleotides in length, generally 19-24 nucleotides in length, and is substantially complementary to at least part of an RNA transcript of a target gene.

The sense strand of an siRNA may include, e.g., 15, 16, 17, 18, 19, 20, 21, or more contiguous nucleotides of the target gene.

In an embodiment, an siRNA can include at least 1, 2, 3, 4, 5, 6, 7, 8, 9, 10, or more modified nucleotides.

In an embodiment, a modified nucleotide can include a 2′-O-methyl modified nucleotide, a nucleotide comprising a 5′-phosphorothioate group, and/or a terminal nucleotide linked to a cholesteryl derivative or dodecanoic acid bisdecylamide group. In an embodiment, a modified nucleotide can include a 2′-deoxy-2′-fluoro modified nucleotide, a 2′-deoxy-modified nucleotide, a locked nucleotide, an abasic nucleotide, 2′-amino-modified nucleotide, 2′-alkyl-modified nucleotide, morpholino nucleotide, a phosphoramidate, and/or a non-natural base comprising nucleotide.

In an embodiment, the region of complementary of a siRNA is at least 10, 11, 12, 13, 14, 15, 16, 17, 18, 19, 20, 21, or more nucleotides in length. In an embodiment, the region of complementary includes 10, 11, 12, 13, 14, 15, 16, 17, 18, 19, 20, 21, or more contiguous nucleotides of the target gene.

In an embodiment, each strand of a siRNA is 15, 16, 17, 18, 19, 20, 21, 22, 23, 24, 25, 26, 27, 28, 29, 30 or more nucleotides in length.

In an embodiment, administration of a siRNA to a cell results in about 40%, 45%, 50%, 55%, 60%, 65%, 70%, 75%, 80%, 90%, 95% or more inhibition of the target mRNA expression as measured by a real time PCR assay. In an embodiment, administration of a siRNA to a cell results in about 40% to 45%, 45% to 50%, 50% to 55%, 55% to 60%, 60% to 65%, 65% to 70%, 70% to 75%, 75% to 80%, 80% to 85%, 85% to 90%, 90% to 95% or more inhibition of the target mRNA expression as measured by a real time PCR assay. In an embodiment, administration of a siRNA to a cell results in about 40%, 45%, 50%, 55%, 60%, 65%, 70%, 75%, 80%, 90%, 95% or more inhibition of the target mRNA expression as measured by a branched DNA assay. In an embodiment, administration of a siRNA to a cell results in about 40% to 45%, 45% to 50%, 50% to 55%, 55% to 60%, 60% to 65%, 65% to 70%, 70% to 75%, 75% to 80%, 80% to 85%, 85% to 90%, 90% to 95% or more inhibition of the target mRNA expression as measured by a branched DNA assay.

In an embodiment, a siRNA has an IC50 of less than 0.01 pM, 0.1 pM, 1 pM, 5 pM, 10 pM, 100 pM, or 1000 pM.

In an embodiment, administration of a siRNA can reduce the target mRNA by about 40%, 45%, 50%, 55%, 60%, 65%, 70%, 75%, 80%, 90%, 95% or more in cynomolgus monkeys. In an embodiment, administration of a siRNA reduces the target mRNA levels by about 40%, 45%, 50%, 55%, 60%, 65%, 70%, 75%, 80%, 90%, 95% or more or serum target protein levels by about 40%, 45%, 50%, 55%, 60%, 65%, 70%, 75%, 80%, 90%, 95% or more. In an embodiment, administration of a siRNA reduces liver target mRNA levels and/or serum target protein levels up to 1, 2, 3, 4, 5, 6, 7, 8, 9, 10, 11, 12, 13, 14, 15, 16, 17, 18, 19, 20, 21, 22, 23, 24, 25, or more days.

Accordingly, in some aspects, pharmaceutical compositions containing an siRNA and a pharmaceutically acceptable carrier, methods of using the compositions to inhibit expression of a target gene, and methods of using the pharmaceutical compositions to treat diseases caused by expression of a target gene are featured.

Gene Editing—CRISPR

In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or other sequences and transcripts from a CRISPR locus. In some embodiments, one or more elements of a CRISPR system is derived from a type I, type II, or type III CRISPR system. In some embodiments, one or more elements of a CRISPR system is derived from a particular organism comprising an endogenous CRISPR system, such as Streptococcus pyogenes. In general, a CRISPR system is characterized by elements that promote the formation of a CRISPR complex at the site of a target sequence (also referred to as a protospacer in the context of an endogenous CRISPR system). In the context of formation of a CRISPR complex, “target sequence” refers to a sequence to which a guide sequence is designed to have complementarity, where hybridization between a target sequence and a guide sequence promotes the formation of a CRISPR complex. Full complementarity is not necessarily required, provided there is sufficient complementarity to cause hybridization and promote formation of a CRISPR complex. A target sequence may comprise any polynucleotide, such as DNA or RNA polynucleotides. In some embodiments, a target sequence is located in the nucleus or cytoplasm of a cell. In some embodiments, the target sequence may be within an organelle of a eukaryotic cell. A sequence or template that may be used for recombination into the targeted locus comprising the target sequences is referred to as an “editing template” or “editing polynucleotide” or “editing sequence”. In aspects of the invention, an exogenous template polynucleotide may be referred to as an editing template. In an aspect of the invention the recombination is homologous recombination.

In particular, the methods described herein may utilize CRISPR/Cas9 system or CRISPR/Cpf1 system. The methods disclosed herein may be used for altering the target gene. A target gene can be altered (e.g., corrected) by gene editing, e.g., using the CRISPR system. The alteration (e.g., correction) of the target gene can be mediated by any mechanism. Exemplary mechanisms that can be associated with the alteration (e.g., correction) of the mutant gene include, but are not limited to, non-homologous end joining (e.g., classical or alternative), microhomology-mediated end joining (MMEJ), homology-directed repair (e.g., endogenous donor template mediated), SDSA (synthesis dependent strand annealing), single strand annealing, or single strand invasion.

For example, the methods and compositions disclosed herein, e.g., a Cas9 or Cpf1 molecule complexed with a gRNA molecule, can be used to target a specific location in a target DNA. Depending on the Cas9 or Cpf1 molecule/gRNA molecule complex used in the methods and compositions, specific editing of a target nucleic acid can be effected.

The CRISPR system typically utilizes a guide RNA (gRNA) for gene editing. The system is targeted to the target sequence by hybridization to crRNA or spacer (e.g., as part of gRNA), which binds to the targeted DNA through base complementarity and provides for precise DNA cleavage. This cleavage is then repaired via various pathways, which can be exploited for different outcomes. Knockouts can be achieved through error prone repair via the Non-homologous End Joining (NHEJ) pathway, which can introduce mutations and disrupt gene function. Targeted integration of a sequence (called a knock-in) can be achieved via the Homology Directed Repair (HDR) pathway, which uses a provided DNA template to repair the cleavage. Activation or repression of a gene can be achieved by targeting catalytically inert Cas9 fused to a transcription activator or repressor to the promoter. gRNA typically includes a spacer and a scaffold, where the former defines the target and the latter is used for Cas-binding. The spacer typically includes about 20 nucleotides complementary to a genomic sequence target. Approaches to designing CRISPR systems are known in the art, see, e.g., U.S. Pat. Nos. 8,697,359 and 10,253,312 and in US 2015/0232881 and US 2018/0187176. gRNA design tools are available for gRNA sequence selection, e.g., at crispr.mit.edu, atum.bio, e-crisp.org, or from ThermoFisher Scientific (thermofisher.com).

Typically, a CRISPR system is introduced into a cell containing and expressing a DNA molecule having a target sequence and encoding the target gene product. Typically, a CRISPR system includes:
a) gRNA that hybridizes to the target gene sequence, and
b) a Cas9 or Cpf1 molecule.

Component (a) may be provided as a first regulatory element operable in a eukaryotic cell operably linked to at least one nucleotide sequence encoding a CRISPR-Cas system gRNA that hybridizes with the target sequence.

Component (b) may be provided as a second regulatory element operable in a eukaryotic cell operably linked to a nucleotide sequence encoding a Cas9 or Cpf1 molecule

Components (a) and (b) may be located on the same or different vectors of the system, whereby the gRNA targets the target sequence and the Cas9 or Cpf1 protein cleaves the DNA molecule, whereby expression of the at least one gene product is altered; and, wherein the Cas9 protein and the guide RNA do not naturally occur together.

A gRNA molecule, as that term is used herein, may refer to a nucleic acid that promotes the specific targeting or homing of a gRNA molecule/Cas9 molecule complex to a target nucleic acid. gRNA molecules can be unimolecular (having a single RNA molecule), sometimes referred to herein as “chimeric” gRNAs, or modular (comprising more than one, and typically two, separate RNA molecules). A gRNA molecule comprises a number of domains. The gRNA molecule domains are described in more detail below. Typically, gRNA will incorporate the functions or structure of both crRNA and tracrRNA, e.g., the functions of processed or mature crRNA and of processed or mature tracrRNA. Chimieric or unimolecular gRNA molecules can have a single RNA molecule, e.g., which incorporates both crRNA function or structure and the tracrRNA function or structure. A modular gRNA molecule can comprise a RNA molecule that incorporates the crRNA function or structure another that incorporates the tracrRNA function or structure.

For CRISPR/Cpf1, crRNA may be used as a gRNA. The Cpf1 protein forms a complex with a single stranded RNA oligonucleotide to mediate targeted DNA cleavage. The single strand guide RNA oligonucleotide consists of a constant region of 20 nt and a target region of 21-24 nt for an overall length of 41-44 nt.

Antibodies

The term “antibody,” as used herein, refers to a monoclonal or polyclonal antibody. A monoclonal antibody includes at least the variable domain of a heavy chain, and normally comprises at least the variable domains of a heavy chain and of a light chain of an immunoglobulin, which bind to an antigen of interest. Antibodies and antigen-binding fragments include, but are not limited to, polyclonal, monoclonal, multispecific, human, humanized, primatized, or chimeric antibodies, single chain antibodies, epitope-binding fragments, e.g., Fab, Fab′ and F(ab′)₂, Fd, Fvs, single-chain Fvs (scFv), single-chain antibodies, disulfide-linked Fvs (sdFv), fragments comprising either a V_{L} or V_{H} domain, fragments produced by a Fab expression library, and anti-idiotypic (anti-Id) antibodies. Antibody molecules of the invention can be of any type (e.g., IgG, IgE, IgM, IgD, IgA, and IgY), class (e.g., IgG1, IgG2, IgG3, IgG4, IgA1 and IgA2) or subclass of immunoglobulin molecule.

An agent of the invention may be an antibody or an antigen-binding fragment thereof. The making and use of therapeutic antibodies against a target antigen (e.g., target gene product) is known in the art. See, e.g., Zhiqiang AN (Editor), Therapeutic Monoclonal Antibodies: From Bench to Clinic. 1st Edition. Wiley 2009, and also Greenfield (Ed.), Antibodies: A Laboratory Manual. (Second edition) Cold Spring Harbor Laboratory Press 2013, for methods of making recombinant antibodies, including antibody engineering, use of degenerate oligonucleotides, 5′-RACE, phage display, and mutagenesis; antibody testing and characterization; antibody pharmacokinetics and pharmacodynamics; antibody purification and storage; and screening and labeling techniques.

Proteins

Any of the proteins described herein as well as any analog or fragment useful as therapeutics. Such proteins also include all mRNA processing variants (e.g., all products of alternative splicing or differential promoter utilization). Specific fragments or analogues of interest include full-length or partial proteins including an amino acid sequence which differs only by conservative amino acid substitutions, for example, substitution of one amino acid for another of the same class (e.g., valine for glycine, arginine for lysine, etc.) or by one or more non-conservative amino acid substitutions, deletions, or insertions located at positions of the amino acid sequence which do not destroy the proteins biological activity. Analogs also include proteins which are modified for the purpose of increasing peptide stability; such analogs may contain, e.g., one or more desaturated peptide bonds or D-amino acids in the peptide sequence or the peptide may be formulated as a cyclized peptide molecule.

Cell Therapies

Using the compositions and methods of the disclosure, a cell (e.g., a human cell) can be modified so as to express a therapeutic gene or gene products and subsequently administered to a subject, such as a subject described herein. Alternatively, or additionally, a cell that expresses one or more gene targets may be induced to replicate ex vivo, and subsequently administered to a subject. Still further, a cell may be induced to differentiate ex vivo into a form that expresses on or more gene targets, and subsequently administered to a subject.

Accordingly, various methodologies can be used to genetically engineer a cell so as to express a desired product. For example, a cell (e.g., a human cell) can be transduced ex vivo to express a desired gene or protein by contacting the cell with a viral vector that encodes the desired target. Viral genomes provide a rich source of vectors that can be used for the efficient delivery of exogenous genes into a cell. Viral genomes are particularly useful vectors for gene delivery, as the polynucleotides contained within such genomes may often be incorporated into the nuclear genome of the target cell, for example, by way of generalized or specialized transduction. These processes occur as part of the natural viral replication cycle, and often do not require added proteins or reagents in order to induce gene integration. Examples of viral vectors that may be used to transduce a cell described herein include, without limitation, a retrovirus, adenovirus (e.g., Ad5, Ad26, Ad34, Ad35, and Ad48), parvovirus, coronavirus, negative strand RNA viruses such as orthomyxovirus (e.g., influenza virus), rhabdovirus (e.g., rabies and vesicular stomatitis virus), paramyxovirus (e.g. measles and Sendai), positive strand RNA viruses, such as picornavirus and alphavirus, and double stranded DNA viruses including adenovirus, herpesvirus (e.g., Herpes Simplex virus types 1 and 2, Epstein-Barr virus, cytomegalovirus), and poxvirus (e.g., vaccinia, modified vaccinia Ankara (MVA), fowlpox and canarypox). Other viruses that may be used to transduce a cell described herein include Norwalk virus, togavirus, flavivirus, papovavirus, hepadnavirus, human papilloma virus, human foamy virus, and hepatitis virus, for example. Examples of retroviruses that may be used to transduce a cell described herein include, without limitation, avian leukosis-sarcoma, avian C-type viruses, mammalian C-type, B-type viruses, D-type viruses, oncoretroviruses, HTLV-BLV group, lentivirus, alpharetrovirus, and gammaretrovirus, spumavirus. Other examples are murine leukemia viruses, murine sarcoma viruses, mouse mammary tumor virus, bovine leukemia virus, feline leukemia virus, feline sarcoma virus, avian leukemia virus, human T-cell leukemia virus, baboon endogenous virus, Gibbon ape leukemia virus, Mason Pfizer monkey virus, simian immunodeficiency virus, simian sarcoma virus, and Rous sarcoma virus.

Another useful tool for the modification of a target cell (e.g., a human cell) to express a desired gene or protein is the clustered regularly interspaced short palindromic repeats (CRISPR)/Cas system, a system that originally evolved as an adaptive defense mechanism in bacteria and archaea against viral infection. The CRISPR/Cas system utilizes palindromic repeat sequences within plasmid DNA, along with a CRISPR-associated protein (Cas; e.g., Cas9 or Cas12a), to cleave endogenous nucleic acids and facilitate the insertion of a gene of interest into a target cell genome. The CRISPR/Cas ensemble of DNA and protein directs site specific DNA cleavage of a target sequence by first incorporating foreign DNA into CRISPR loci. Polynucleotides containing these foreign sequences and the repeat-spacer elements of the CRISPR locus are, in turn, transcribed in a host cell to create a guide RNA, which can subsequently anneal to a target sequence and localize the Cas nuclease to this site. One can design site-specific CRISPR/Cas constructs because the interaction that brings the Cas nuclease within close proximity of the target DNA molecule is governed by RNA:DNA hybridization. As a result, one can design a CRISPR/Cas system to cleave any target DNA molecule of interest. This technique has been exploited in order to edit eukaryotic genomes (Hwang et al. Nature Biotechnology 31:227 (2013), the disclosure of which is incorporated herein by reference) and can be used as an efficient means of site-specifically editing cells described herein so as to express a gene of interest.

Additionally or alternatively, a cell of the disclosure (e.g., a human cell) may be modified ex vivo so as to express a desired gene or protein by contacting the cell with a polynucleotide containing the desired gene or encoding the desired protein, for example, under conditions that promote the uptake of the polynucleotide by the cell. Examples of such methods include, without limitation, calcium phosphate precipitation, electroporation, microinjection, infection, and lipofection. Such methods are described in more detail, for example, in Green et al., Molecular Cloning: A Laboratory Manual, Fourth Edition (Cold Spring Harbor University Press, New York (2014)); and Ausubel et al., Current Protocols in Molecular Biology (John Wiley & Sons, New York (2015)), the disclosures of each of which are incorporated herein by reference.

Recognition and binding of the polynucleotide encoding one or more therapeutic proteins of the disclosure by mammalian RNA polymerase is important for gene expression. As such, one may include sequence elements within the polynucleotide that exhibit a high affinity for transcription factors that recruit RNA polymerase and promote the assembly of the transcription complex at the transcription initiation site. Such sequence elements include, e.g., a mammalian promoter, the sequence of which can be recognized and bound by specific transcription initiation factors and ultimately RNA polymerase. Examples of mammalian promoters have been described in Smith et al., Mol. Sys. Biol., 3:73, online publication, the disclosure of which is incorporated herein by reference.

Once a polynucleotide encoding one or more therapeutic proteins has been incorporated into the nuclear DNA of a mammalian cell, transcription of this polynucleotide can be induced by methods known in the art. For example, expression can be induced by exposing the mammalian cell to an external chemical reagent, such as an agent that modulates the binding of a transcription factor and/or RNA polymerase to the mammalian promoter and thus regulates gene expression. The chemical reagent can serve to facilitate the binding of RNA polymerase and/or transcription factors to the mammalian promoter, e.g., by removing a repressor protein that has bound the promoter. Alternatively, the chemical reagent can serve to enhance the affinity of the mammalian promoter for RNA polymerase and/or transcription factors such that the rate of transcription of the gene located downstream of the promoter is increased in the presence of the chemical reagent. Examples of chemical reagents that potentiate polynucleotide transcription by the above mechanisms are tetracycline and doxycycline. Such reagents are commercially available and can be administered to a mammalian cell in order to promote gene expression according to established protocols.

Other DNA sequence elements that may be included in polynucleotides for use in the compositions and methods described herein are enhancer sequences. Enhancers represent another class of regulatory elements that induce a conformational change in the polynucleotide containing the gene of interest such that the DNA adopts a three-dimensional orientation that is favorable for binding of transcription factors and RNA polymerase at the transcription initiation site. Thus, polynucleotides for use in the compositions and methods described herein include those that encode one or more therapeutic proteins and additionally include a mammalian enhancer sequence. Many enhancer sequences are now known from mammalian genes, and examples are enhancers from the genes that encode mammalian globin, elastase, albumin, α-fetoprotein, and insulin. Enhancers for use in the compositions and methods described herein also include those that are derived from the genetic material of a virus capable of infecting a eukaryotic cell. Examples are the SV40 enhancer on the late side of the replication origin (bp 100-270), the cytomegalovirus early promoter enhancer, the polyoma enhancer on the late side of the replication origin, and adenovirus enhancers. Additional enhancer sequences that induce activation of eukaryotic gene transcription are disclosed in Yaniv et al., Nature 297:17 (1982).

In other embodiments, expression may be constitutive. Such constructs are engineered according to methods known in the art.

Other DNA sequence elements that may be included in polynucleotides for use in the compositions and methods described herein affect the stability and/or splicing of the transcribed mRNA and/or stability of the translated protein product. Examples include sequences derived from FKBP12 which destabilize proteins in the absence of exogenous ligands (Banaszynski et al. Cell 126:995 (2006)).

An example of a cell therapy is modifying hematopoietic stem cells to express the beta-globin protein, which is defective in sickle cell anemia patients, and transplanting the modified cells into patients as a treatment for sickle cell anemia (Clinical trial NCT02140554). Another example is modifying T cells to express a chimeric antigen receptor that enables them to respond to tumor cells, and administering these modified cells to cancer patients, as described in Newick et al., Annual Review of Medicine 68:139 (2017).

### Examples

The invention is now described with reference to the following Examples. These Examples are provided for the purpose of illustration only, and the invention is not limited to these Examples, but rather encompasses all variations which are evident as a result of the teachings provided herein.

### Methods

For the Examples described below the following classes were studied:
Resistant species: mice, rats, rhesus monkeys (rhesus macaque), baboons; and
Sensitive species: rabbits, pigs, cow, sheep, chimpanzees, and humans.

In every case, genes or proteins with significant differences in expression or regulation between resistant species and sensitive species were identified.

We now described Experimental Designs for (1) whole blood transcriptome and plasma proteome and (2) endothelial studies.

### Whole Blood Transcriptome and Plasma Proteome Studies

Blood was drawn from each of the above species (mice, rats, rhesus monkeys (rhesus macaque), baboons, rabbits, pigs, cow, sheep, chimpanzees, and humans) into prepared “TruCulture” tubes containing heparin and one of three pre-adjusted concentrations of ligands to make final concentrations of the national reference endotoxin E. coli 0113 LPS (10, 100, and 1000 ng/ml) and Poly I:C (10,000 ng/ml) according to standard procedures. The blood was incubated for different times (2 h, 6 h, 24 h), after which the cells were separated, the plasma removed and saved, the red cells lysed, and the remaining cells washed, frozen and sent to Expression Analysis, Inc (Durham, N.C.) for mRNA sequencing. Plasma proteins were processed and analyzed using a liquid chromatography mass spectrometry (LC-MS) platform, in which peptide level analysis was used to identify and quantify specific proteins within each sample and species. Sample processing involves isolation of the protein component of the plasma followed by reduction and digestion to the peptide form which was analyzed within the LC-MS platform. The following analyses were performed:

### Whole Blood Transcriptome Analysis

Sequence reads were aligned to the appropriate ENSEMBL genome assembly v90 and expressed as transcripts per million. Genes from each species were then assigned to their human orthologs, as defined by ENSEMBL. Genes without human orthologs were eliminated. Where gene loss or duplication resulted in multiple paralogs, the read counts were applied to all members of the family; the evolutionary mismatches were noted and taken into consideration during interpretation. Two sets of analysis were performed, always comparing expression in resistant species with expression in sensitive species.

A. Baseline Differences

In this approach, we identified intrinsic differences between gene expression in sensitive vs. resistant species that did not necessarily depend on stimulation. Only samples incubated for 2 hours without LPS stimulation were considered. This condition most closely reflects conditions in the healthy animal, so is referred to as “baseline”. We identified genes that showed clear differences in expression between all sensitive and all resistant species. This is usually defined as FDR <0.05 between mean expression in the two classes, a 1.5-fold difference in expression between all species in each class, and the gene being present in all but one members of the higher expressing class. However, some flexibility in analysis criteria was employed according to genomic context, evolutionary conservation, understanding of bioinformatic methods, and existing knowledge about specific genes.

B. Fold Change Differences

This approach identified differences in the responses to stimulation, as opposed to intrinsic differences at baseline. Fold change on LPS stimulation was calculated by dividing a gene's expression after stimulation with LPS by the expression after incubation for the same length of time without LPS. Targets are selected from either or both of two different analyses.

In the first analysis, significant (FDR<0.05, fold difference >2) difference was required between the mean fold change across sensitive species and the mean fold change across resistant species at 10 pg/ml LPS at any of the three times. These genes were then manually reviewed prior to inclusion. In the second analysis, pigs were counted as resistant for phylogenetic reasons. In this, approach statistical significance was required in all pairwise comparisons of resistant and sensitive species at any time and any dose of LPS.

### Proteomic Analysis

LC-MS data provided a quantitative peak value for each specific peptide sequence which was linked to the relevant protein, and from which quantitative protein data was generated. Proteins were identified as prospective mediators of resistance or sensitivity if they either 1) achieved a p value <0.05 by ANOVA testing, 2) were detected in at least 3 species of one class but were not detected in any species of the other class, or 3) were not detected in enough species of each class to allow statistical treatment, but nonetheless achieved a >2-fold difference in abundance between classes.

### Validation Assays

### Whole Blood Cytokine Release

Fresh, anonymized, heparinized, human blood from male donors was diluted 1:3 with RPMI containing penicillin and streptomycin. 150 μl diluted blood was added to each well of a 96 well plate then acclimatized to 37° C. and 5% CO₂ for 1 hour. Inhibitors were prepared as described below then added to the diluted blood in 25 μl volume. After a further hour at 37° C. with 5% CO₂, diluted blood was stimulated with LPS diluted in 25 μl RPMI containing penicillin and streptomycin to a final concentration of 1.5 ng/ml. After 18 h at 37° C. and 5% CO₂, diluted blood was centrifuged at 500×g for 5 min and supernatant collected and stored at −20° C. until ELISAs were performed. Data are presented as mean inhibition relative to vehicle control ±95% confidence intervals, n=3-4 independent experiments.

Inhibitors and vehicles used for resuspension were: Compound 26, EMD Millipore, Cat #5.31237.0001, resuspended in DMSO; PF 06650833, R & D Systems, Cat #6373, resuspended in DMSO; OEPC (oleyloxyethyl phosphorylcholine), Santa Cruz Biotechnology, item # sc-200711, resuspended in DMSO; YM26734, Santa Cruz Biotechnology, item # sc-204410, resuspended in DMSO; Isoquercetin, Cayman Chemical, item #24926, resuspended in DMSO; ISA-2011B, Medchem Express, cat # HY-16937, resuspended in DMSO; Benzamil, TOCRIS, Cat #3380/10, resuspended in water; Cell permeable NFAT inhibitor (11R-VIVIT), Tocris, Cat #5710, resuspended in PBS. ELISAs were performed using Duoset reagents from R&D systems, catalogue # DY210 (human TNF), # DY206 (human IL6), and #DY201 (human IL-1β).

### RNAi

PBMCs were isolated from fresh, heparinized, anonymized, human blood by centrifugation on Ficoll 1077 (Sigma) then washed twice with RPMI without antibiotic. PBMCs were resuspended to 1.75×106 per ml in OPTI-MEM I (Gibco), and plated at 175,000 cells per well of a 96 well plate. Cells were transfected by adding 1 μl×100 μM Accell self-delivering siRNA (Horizon Discovery) and maintained at 37° C. with 5% CO2. Catalog numbers for Accell siRNAs were as follows: 23098 (SARM1), 51135 (IRAK4), 7099 (TLR4), D-001910-10-20 (non-targeting pool). After approx. 48 hours, 150 μl RPMI containing 10% FCS was added ±E. coli LPS to a final concentration of 2.5 ng/ml. After a further 4 hours, plates were centrifuged at 500×g for 5 min. 200 μl supernatant was removed and stored at −20° C. until ELISA as described above.

### Endothelial Studies

Exposure of the endothelium to bacterial cells or to LPS results in leakage of plasma into tissues. This finding can be mimicked in vitro using systems that measure the passage of macromolecules or electrical conductance (transepithelial electrical resistance, TER) across a monolayer of endothelial cells.

First, using electric cell-substrate impedance sensing (ECIS), we found that the TER across a monolayer of lung microvascular endothelial cells (LMVECs) from sensitive species drops markedly following stimulation with LPS or heat-killed E. coli 018 K+(HKEC), but LMVECs from resistant species show a much more modest change in TER. This finding is consistent with sensitive species suffering greater plasma leakage.

Second, we also found that human LMVECs grown in serum from resistant species experience a smaller drop in TER than human LMVECs grown in serum from sensitive species. This is consistent with our observation that endotoxin sensitivity of PBMCs depends in part on the serum in which they are grown, with serum from resistant species conferring resistance on cells PBMCs from sensitive animals.

Third, we found that human LMVECs pretreated with HKEC show much reduced sensitivity to subsequent stimulation with HKEC or LPS—this is an example of tolerance.

### Transcriptomic Profiling

To identify candidate mediators of resistance, we performed transcriptomic profiling on these three parings of sensitive and resistant phenotypes, and then identified genes that were either (i) expressed differently at baseline (in the absence of stimulation) or (ii) responded differently to stimulation with LPS or HKEC.

As a form of internal validation, and so as to identify central mediators of endotoxin resistance rather than specific regulators of just one process, we required a significant difference in at least two of the three comparisons for inclusion. Furthermore, the difference had to be in the same direction between different comparisons, viz. expressed more highly in resistant than sensitive in both cases, or vice versa.

1. Comparison of Response of Endothelial Cells from Sensitive and Resistant Species

For this analysis, the following species were used:
Resistant species cells: rat, mouse; and
Sensitive species cells: human, cow.

LMVECs were purchased from PromoCell (human), Alphabioregen (cow), and iXCells (rat). Mouse LMVECs were purchased from Cell Biologics or prepared according to standard methods.

Cells were cultured in EGM-2 MV (Lonza) supplemented with 5% fetal calf serum (FCS) until confluent, then seeded into 12-well plates at 10⁵ cells per well. After a further 3-4 days, when cells were near confluence as assessed by microscopy, LPS and HKEC were added to wells to final concentrations of 0.1 pg/ml and 10⁶ CFU/ml respectively. After 1 or 3 hours, wells were washed once with PBS, lysed using 1 ml Qiazol (Qiagen) and stored at −80° C. Transcriptomic analyses were performed by Expression Analysis. Each condition was performed in triplicate.

RNAseq reads were aligned, quantified, and matched to human genes as for whole blood RNAseq (above). Genes with significantly (FDR <0.05, 2-fold tpm difference) different expression between both sensitive and both resistant species with or without HKEC stimulation at either timepoint, and genes with significantly different responses to stimulation at either timepoint were identified.

2. Study of Human Endothelial Cells Cultured in Serum from Sensitive and Resistant Species

For this analysis, the following sera was used:

Resistant species serum: rat, mouse; and Sensitive species serum: human, cow (fetal calf), rabbit.

We also used sera from the following species: human (prepared in lab), Sprague Dawley rats (prepared in lab), mouse (Equiptech-Bio), New Zealand White Rabbits (prepared in lab), fetal calf serum (Lonza). Human LMVEC were pre-incubated for 1-3 days in EGM-2 supplemented with the specific serum (human, fetal calf, rabbit, mouse, rat) at a concentration of 5% serum. Cells were then harvested, washed, resuspended in EGM-2 with the same species' serum and utilized for plated in 12 well dishes. After 2-3 days, when cells were near confluence as assessed by microscopy, cells were stimulated, harvested, and sequenced as above. The experiment was repeated 3-4 times to control for batch and donor effects. Sequencing reads were aligned and quantified, and statistical analysis was performed in 1 (Comparison of response of endothelial cells from sensitive and resistant species) as above.

3. Comparison of Normal and Tolerant Human Endothelial Cells

For this analysis, the following phenotypes were used:

Resistant phenotype: human LMVECS pretreated with HKEC; and

Sensitive phenotype: human LMVECs without pretreatment.

Human LMVEC were grown in EGM-2 with 5% FCS until confluent, then stimulated with or without 10⁸ HKEC per T75 flask for 6 hours. LMVECs were then washed, detached, and seeded in 12 well plates at 10⁵ cells per well. After 2-3 days, when the monolayer was approaching confluence, wells were stimulated with or without LPS or HKEC for 1 or 3 hours then harvested and sequenced, as above. Each condition was performed in triplicate.

Sequencing reads were aligned and quantified, and statistical analysis was performed as in 1(Comparison of response of endothelial cells from sensitive and resistant species) above. In addition, to remove a potential confounding effect of pretreatment, HKEC response genes where pre-treatment with HKEC appeared to enhance the response to second stimulation were excluded.

### Validation Assays

Where direct chemical modulators of the proposed inflammatory modulators were available, we tested their effect on endothelial permeability using ECIS and/or on the leukocyte inflammatory response using the whole blood assay described above.

Electric cell substrate impedance sensing (ECIS; Applied Biophysics) measures transendothelial resistance (TER) to the flow of electrical current as a surrogate for permeability. Human LMVECs were seeded into wells of 96-well electrodes (50,000 cells/well). When cells reached confluence, they were pre-treated with the indicated inhibitor or vehicle for 1 hour then stimulated with LPS (0.01-1 pg/ml) or HKEC (10⁵-10⁸ CFU/ml). TER was measured repeatedly over a 24 hours period.

Agents used were SJ000291942 (Sigma #SML2087-5MG) and saxagliptin (Santa Cruz Biotechnology #sc-473161), both resuspended in DMSO.

### Results

Results are presented below according to the experiment and analysis approach. Some of the targets identified by each approach have existing links to inflammatory disease while others are previously unidentified. The identification of known mediators not only validates our platform but also validates our identification of the previously unknown targets described herein. Where reagents are available, we have performed proof-of-principle experiments.

A. Whole Blood Transcriptome Analysis—Results from BASELINE Analysis

Forty-three (43) genes are expressed at different levels in whole blood (leukocytes) from sensitive and resistant species without respect to LPS stimulation, indicating intrinsic differences between the species. These intrinsic differences in gene expression may contribute to the intrinsic differences in LPS between species, and that compounds or methods that alter the expression of these genes or the activities of their products constitute novel therapies. This approach is strongly supported by our identification of known mediators of LPS signaling and/or inflammation, including at least one established anti-inflammatory drug target, which increases our confidence in the significance of the other targets identified by our method.

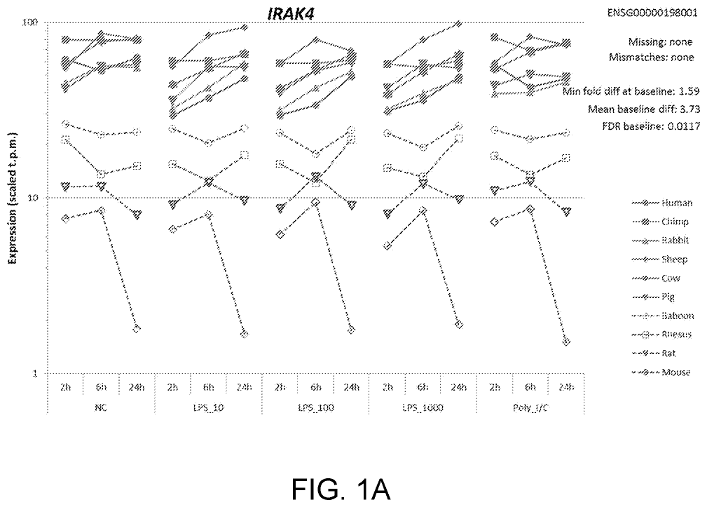

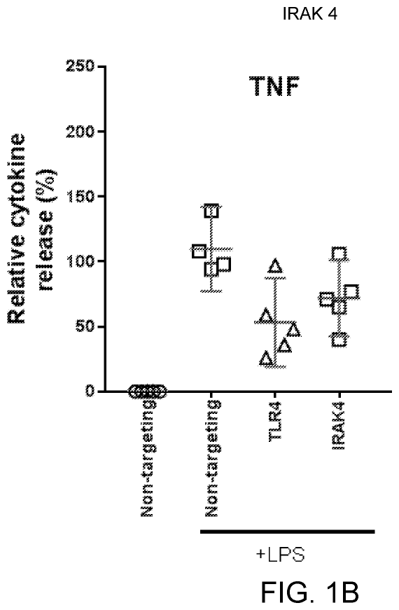

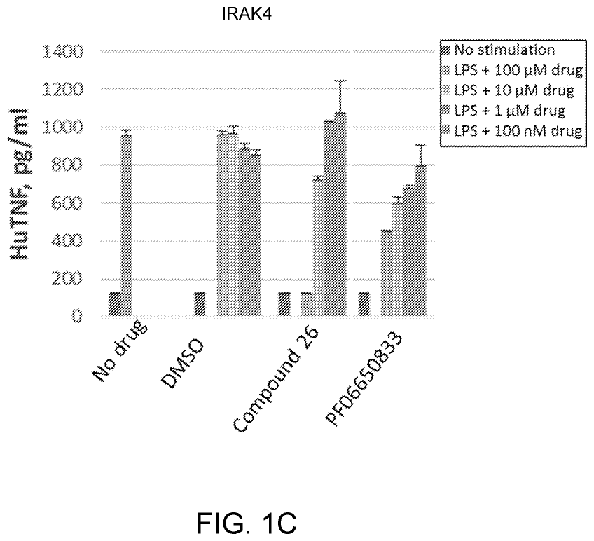

For each target, the gene expression profile (in FIG. 1 through FIG. 43) is given for all species for all time and stimulation conditions used. However, the analysis focused on the condition at the far left: 2 hour incubation without stimulant. We call this the baseline condition, which most closely reflects the basal expression within the living animal. In each graph, resistant species are noted throughout. Mean mRNA expression across 3-5 animals is plotted. The FDR corrected p-value for separation between sensitive and resistant, minimum fold difference (fold difference between expression in sensitive species that expresses most strongly and resistant species that expressed most weakly, or vice versa, as appropriate), and average fold difference (fold difference between the average expression in sensitive species and average expression in resistant species) at baseline (2 hour, no stimulation) is given. Some genes were not annotated in the genome assemblies used or have been duplicated or lost during evolution resulting in mismatches between humans and some species—this is noted using the abbreviations Ch (chimp), Rh (rhesus). B, (baboon), C (cow), Sh (sheep), P (pig), M (mouse), Rt (rat) and Rb (rabbit). The ENSEMBL database accession number (ENSGxxx) for the gene is given.

Where chemical inhibitors are available, the effect of these on cytokine secretion in whole blood assays is shown as percent inhibition. In some cases, the effect of mRNA knockdown in PBMCs on cytokine secretion is shown.

### IRAK4

IRAK4 is a known regulator of inflammatory signaling. It links the TLR adaptor protein Myd88 to other members of the myddosome, and is therefore essential for MAP kinase and IκB activation. It is a target for inflammatory disease, with at least two small molecule inhibitors in clinical trials. Its discovery and successful validation in our system provides evidence of the validity and effectiveness of the methods disclosed herein for target identification (FIGS. 1A, 1B, and 1C).

### TREML1

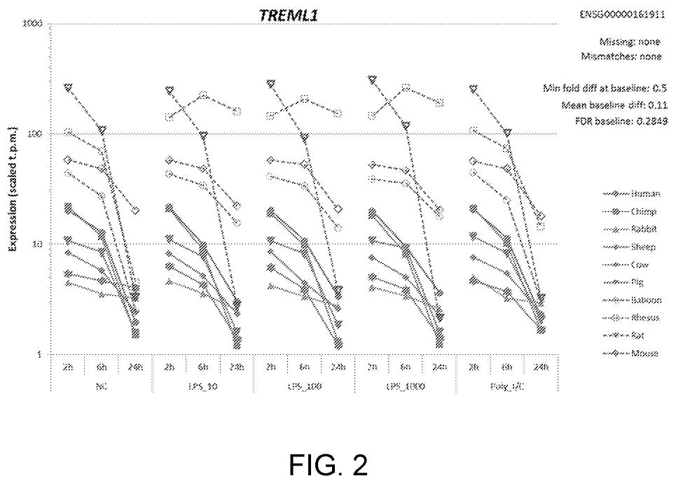

TREML1 is a target for sepsis. It is believed to be secreted by platelets as a competitive inhibitor that prevents the myeloid receptor TREM1 from binding to an unidentified proinflammatory signaling molecule. A peptide mimetic of the putative ligand binding region, which is shared between TREM1 and TREML1 has shown benefit in animal models of endotoxin toxicity and is in clinical trial. TREML1 expression inversely correlates with risk of developing Alzheimer's disease, which is believed to have an inflammatory component. Its identification in our analysis supports the robustness of the disclosed methods to identify correctly mediators of inflammatory disease in the wider sense, and not just immediate participants in LPS signaling (FIG. 2).

### NFATC2

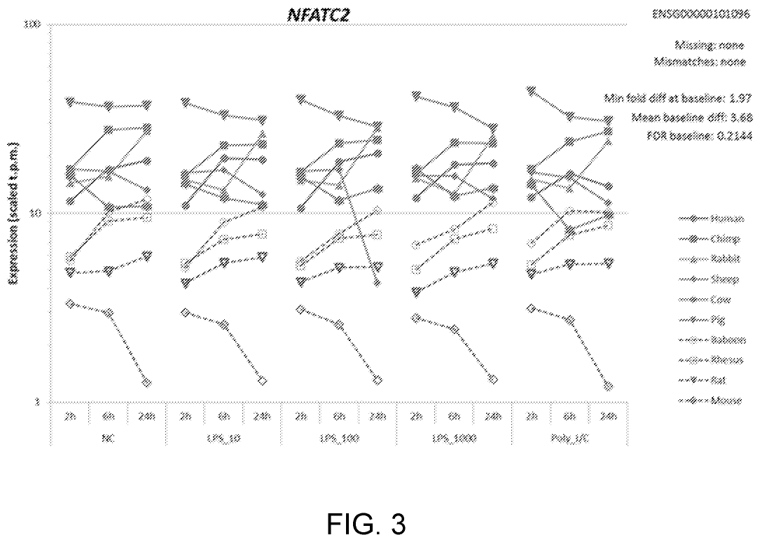

Nuclear factor of activated T cells family members are well established immune mediators. The identification of NFATC2 in our platform (FIG. 3) and its confirmation in the results found in Table 1 below using a pan-NFAT competitive inhibitor (11R-VIVIT) supports the validity of the methods disclosed herein.

TABLE 1

| | % Inhibition of cytokine release, relative to vehicle | | |
|---|---|---|---|
| 11R-VIVIT | TNF | IL6 | 1L1β |
| 50 μM | 79% ± 14.3% | 87% ± 17.9% | 51% ± 26.3% |
| 25 μM | 40% ± 7.1% | 2% ± 32.9% | 48% ± 27.4% |
| 12.5 μM | 23% ± 12.2% | −10% ± 16.7% | 32% ± 13.0% |

### PLA2G10

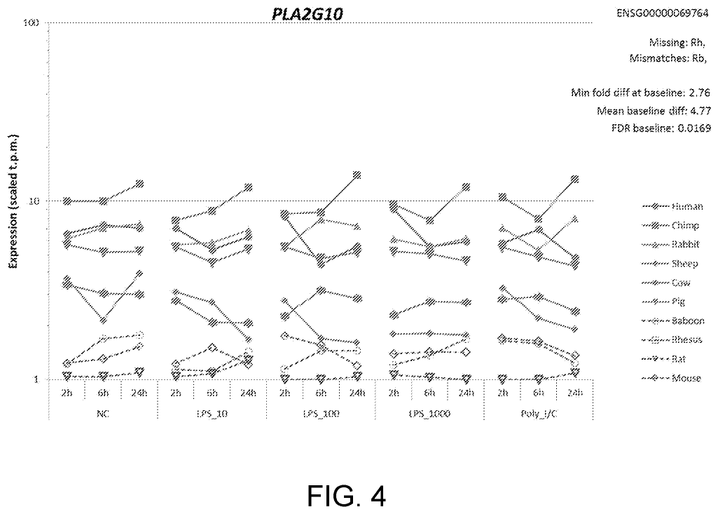

PLA2G10 encodes sPLA2-X a secreted member of the phospholipase A2 family. As such, it has the potential to influence production and metabolism of the known inflammatory mediators: prostaglandin and platelet activating factor. Unlike most other PLA2 enzymes, sPLA2-X is secreted into the bloodstream, where it can both modify inflammation-modulating lipoproteins and act directly on cell surface receptors. Modulation of PLA2G10 or its gene product provides a useful means of treating disorders of immune dysregulation described herein. (FIG. 4). We show, in Table 2 below, that two sPLA2-X inhibitors, OBAA and YM26734, effect dose-dependent inhibition of proinflammatory cytokine release.

TABLE 2

| | % Inhibition of cytokine release, relative to vehicle | | | | |
|---|---|---|---|---|---|
| Drug | OEPC | | YM26734 | | |
| concentration | TNF | IL6 | TNF | IL6 | IL1β |
| 100 μM | 57% ± 6.5% | 83% ± 6.2% | 52% ± 22.7% | 67% ± 15.1% | 78% ± 8.9% |
| 50 μM | 37% ± 14.7% | 65% ± 11.7% | 64% ± 26.1% | 67% ± 21.7% | 73% ± 11.3% |
| 25 μM | 6% ± 17.4% | 39% ± 14.1% | 55% ± 22.6% | 51% ± 29.5% | 65% ± 3.5% |
| 12.5 μM | −9% ± 17.3% | −3% ± 17.6% | 41% ± 17.6% | 35% ± 18.8% | 51% ± 12.0% |

### SARM1

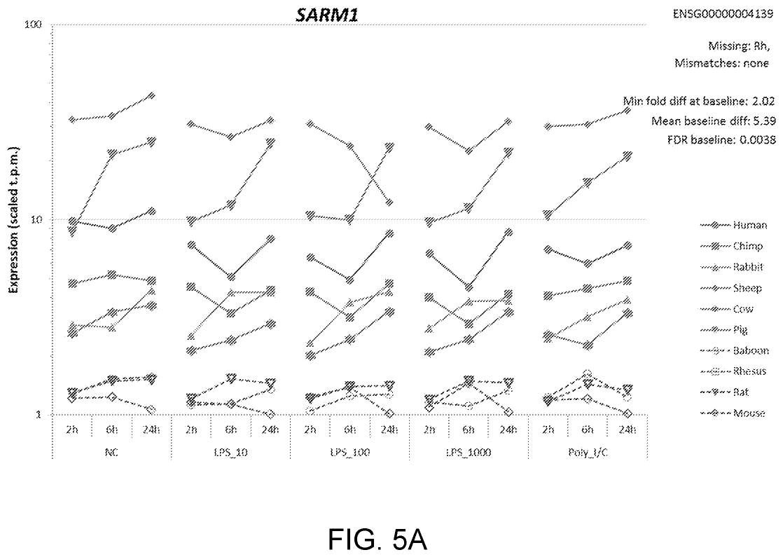

SARM1 encodes SARM, the 5th member of the TLR signalling adaptor family that also includes Myd88, TRIF, TRAM, and MAL. It has been shown to interact with Myd88 in vitro. This places it at a key location in the proinflammatory signal transduction pathway from TLR and IL1β receptors. Molecules that affect its activity are accordingly useful regulators of inflammation. The role of SARM in axon degeneration and neuron survival has been documented and is under development but its role in leukocyte inflammatory signaling has been neglected. Early reports showed that overexpression in HEK293 cells inhibited TLR signaling, but this has been contradicted in bone marrow derived macrophages from knockout mice. It is unclear whether the discrepancy reflects context specific roles or overexpression artefacts in the earlier studies. A recent report proposed that SARM mediates inflammatory cell death following inflammasome activation in murine macrophages, and that this cell death mediates the lethality of murine endotoxemia. However, this report showed that TNF secretion in murine Sarm1 knockout macrophages was no different to wild-type. Its identification using the methods disclosed herein and validation by gene knockdown indicates a role for SARM1 in human inflammation, modulation of which by established methods may underpin new immunomodulatory therapies (FIGS. 5A, 5B).

### PIP5K1A

PIP5K1A encodes phosophinositol phosphate 5-kinase 1α: an enzyme that influences receptor signaling and internalisation, including CD14 and TLR4. Its phosphorylated product, PI(4,5)P2 enables recruitment of TIRAP to the plasma membrane and the initiation of Myd88 dependent signalling. Modulation of its activity is accordingly useful for modulating the immune response.

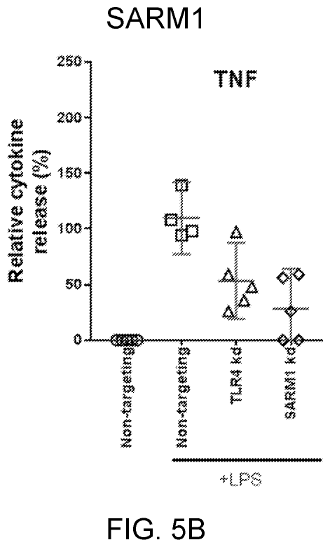

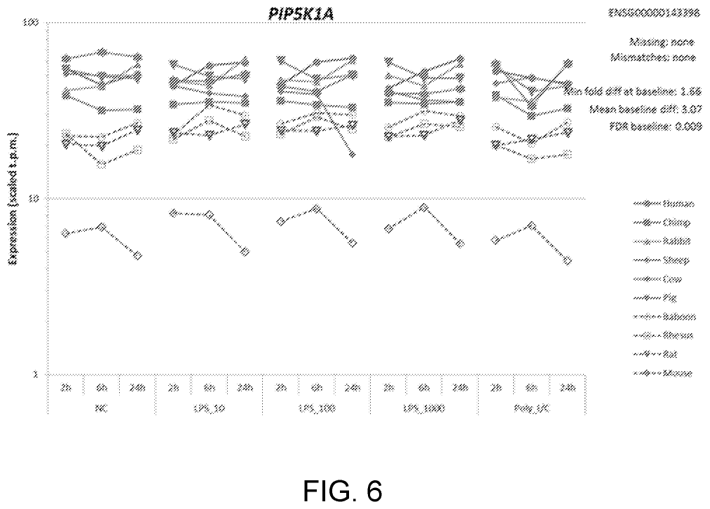

Its identification by the methods disclosed herein indicates a role for PIP5K1A in human inflammation (FIG. 6).

In the table below, we demonstrate that a specific PIP5K1a inhibitor, ISA-2011B (see, e.g., Semenas et al. Proc. Nat'l Acad. Sci. USA 111 (35) E3689-E3698 (2014)), inhibits cytokine release in whole blood assay in response to LPS stimulation in a whole blood assay. ISA-2011B was designed as an anti-cancer agent rather than an anti-inflammatory agent, by inhibiting PIP5K1α-dependent growth factor signaling. The findings below (Table 3) provide indicate that inhibitors of PIP5K1a are also useful as anti-inflammatory agents.

TABLE 3

| | % Inhibition of cytokine release, relative to vehicle | | |
|---|---|---|---|
| ISA-2011B | TNF | IL6 | 1L1β |
| 100 μM | 55% ± 8.8% | 24% ± 19.5% | 86% ± 2.9% |
| 10 μM | 17% ± 7.6% | 6% ± 19.7% | 23% ± 6% |
| 1 μM | 1% ± 11.2% | −7% ± 7.7% | 17% ± 9% |

### PDIA4

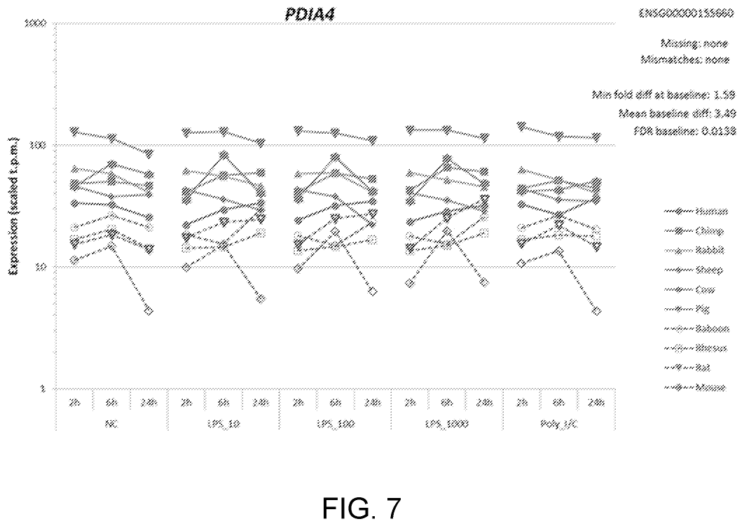

PDIA4 encodes a protein disulfide isomerase, Erp72. It is released by activated platelets and contributes to thrombus formation. It is further useful as a gene target for inflammatory disease. A chemical inhibitor, isoquertecin, showed marginal effect in our in vitro whole blood cytokine release assay (Table 4). This is the only target for which we were able to obtain inhibitors which did not validate persuasively (FIG. 7). It is unclear at this stage whether this indicates a genuine false positive from our platform, poor specificity or other failing in the inhibitor, or a role in inflammation that is not well captured by our whole blood assay. The reported and projected roles of Erp72 in clotting and cell adhesion supports this latter option.

TABLE 4

| | % Inhibition of cytokine release, relative to vehicle | | |
|---|---|---|---|
| Isoquertecin | TNF | IL6 | IL1β |
| 100 μM | 29% ± 10.7% | 26% ± 19.2% | 19% ± 35.6% |
| 50 μM | 23% ± 4.6% | 31% ± 8.6% | 25% ± 17.5% |
| 25 μM | 9% ± 1.1% | 26% ± 6.2% | 15% ± 15.6% |

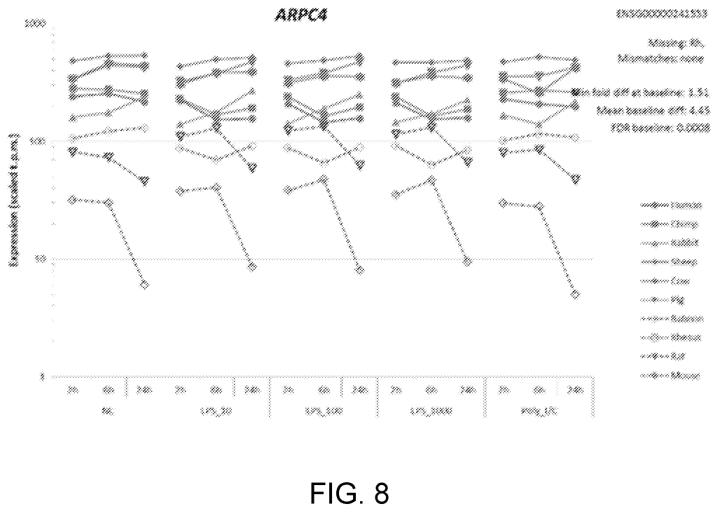

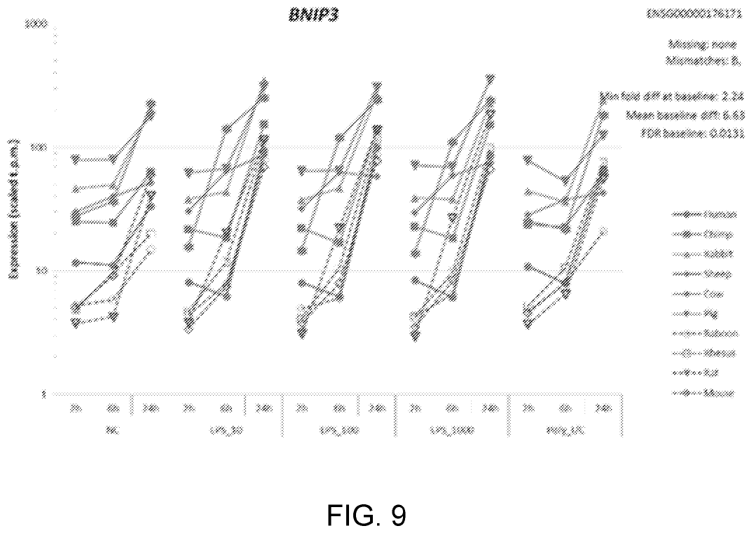

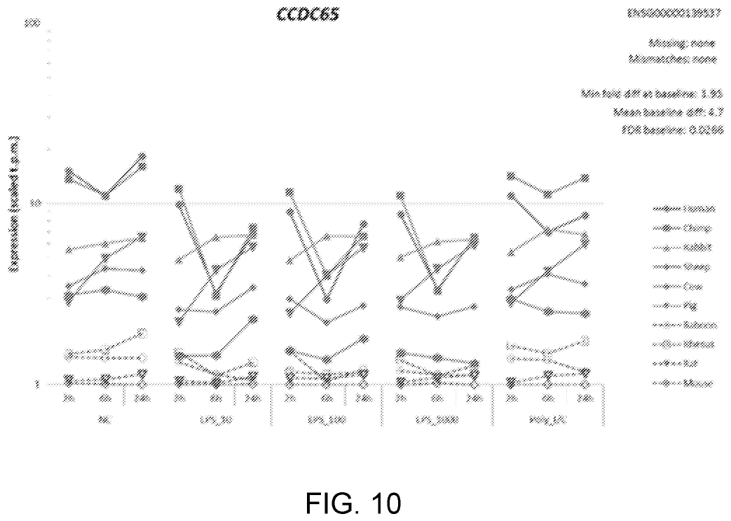

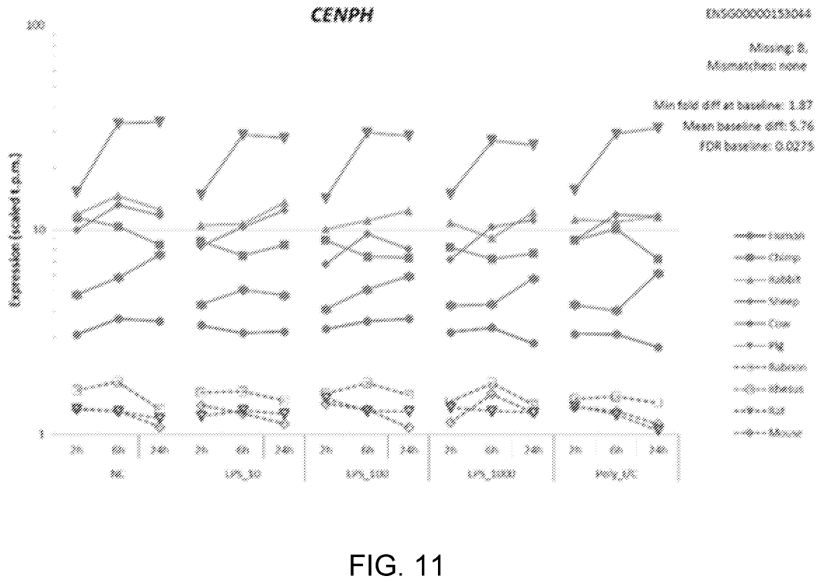

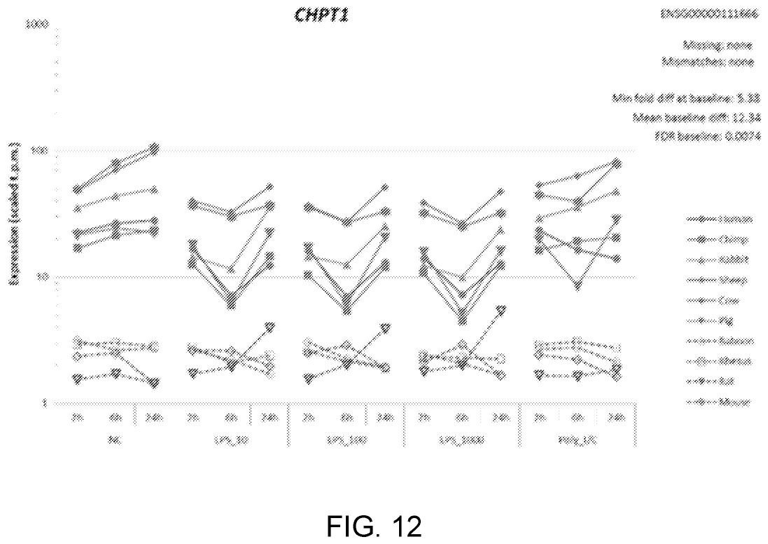

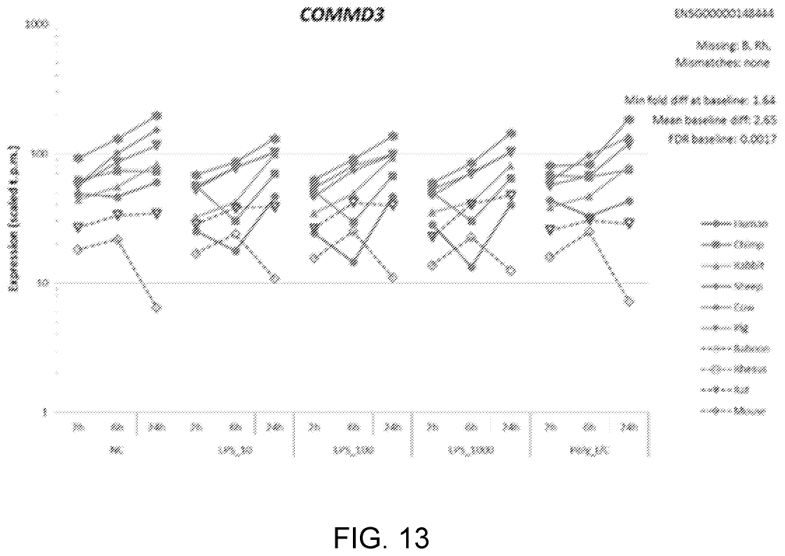

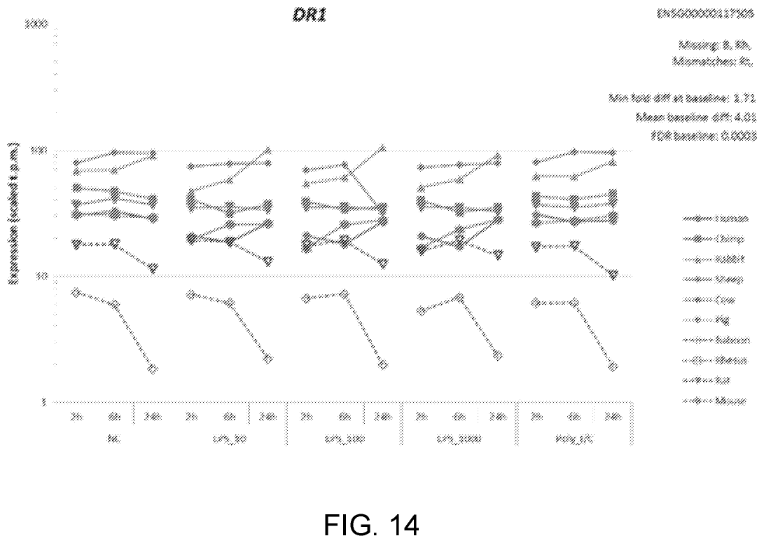

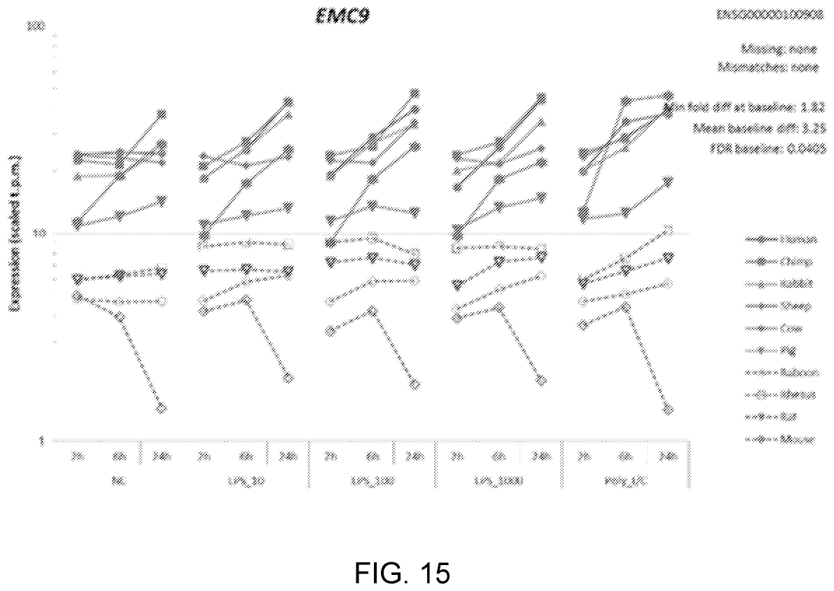

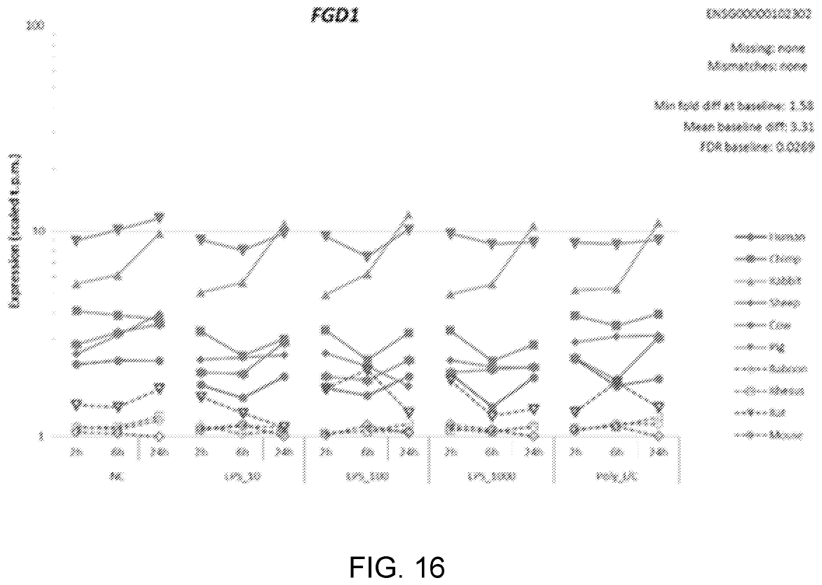

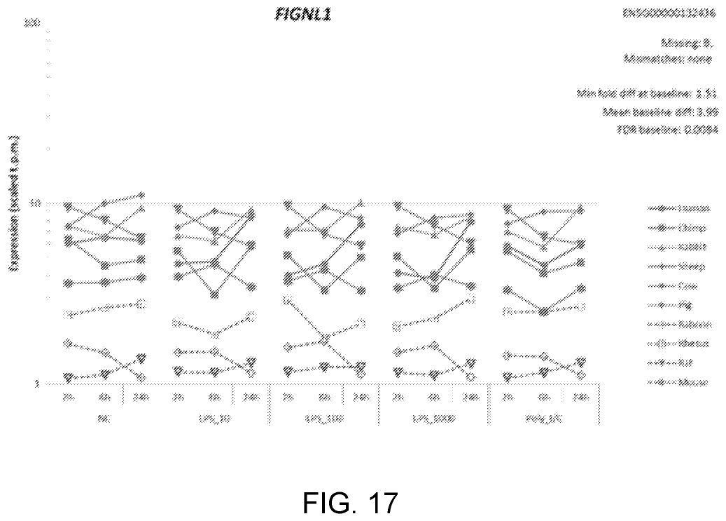

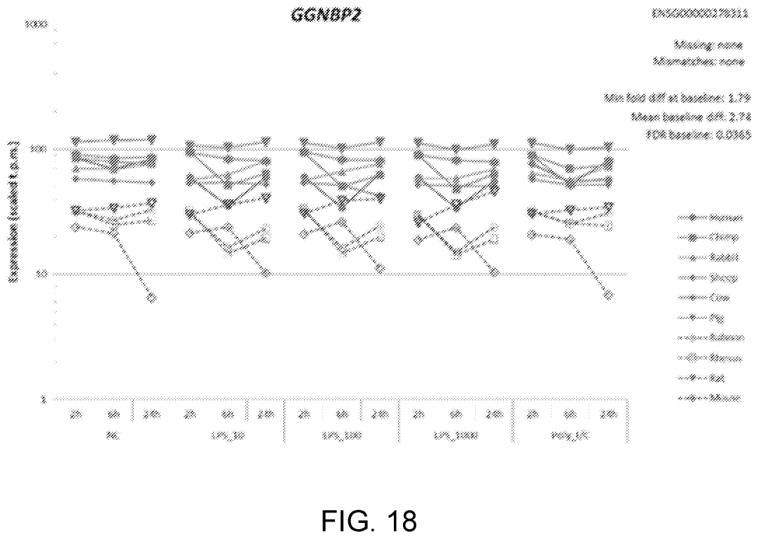

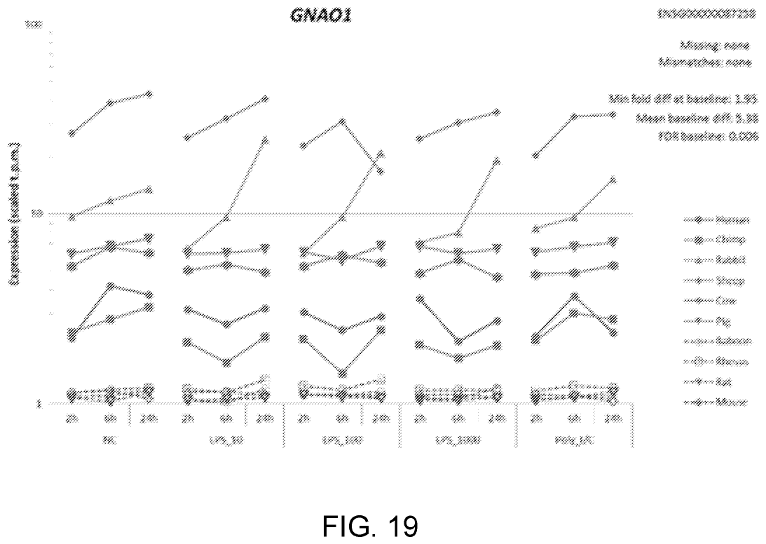

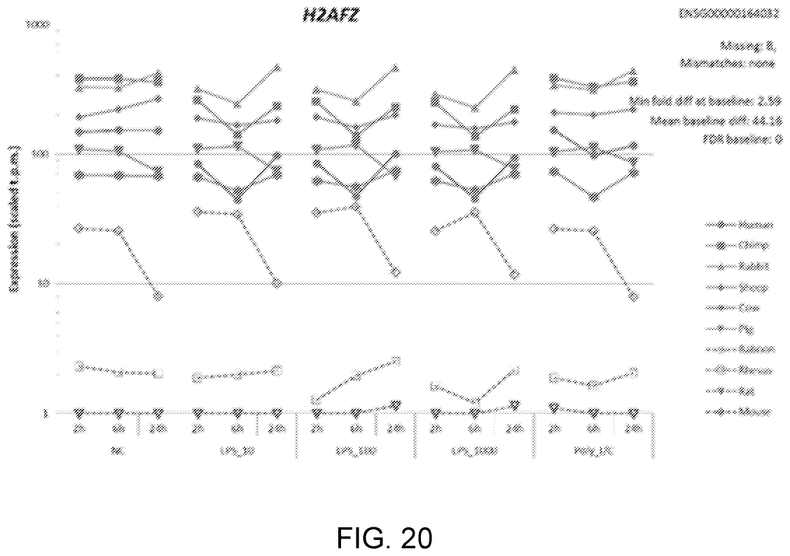

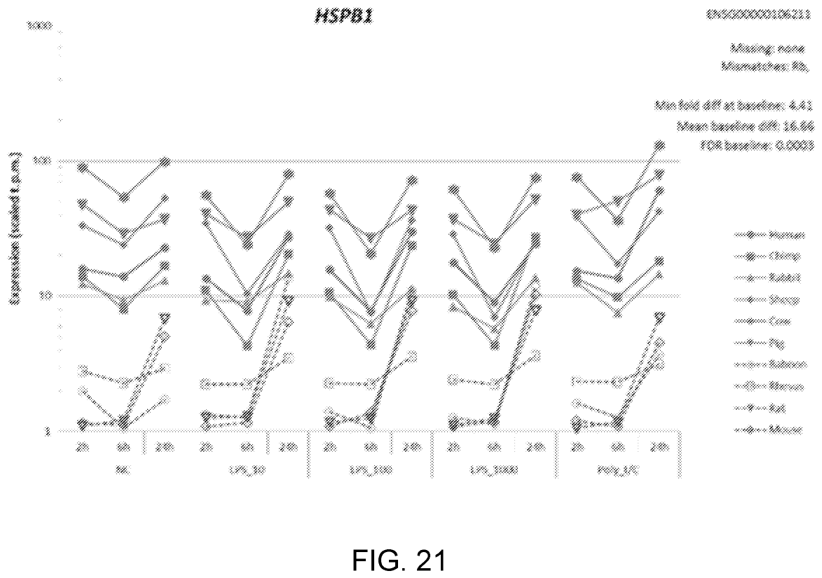

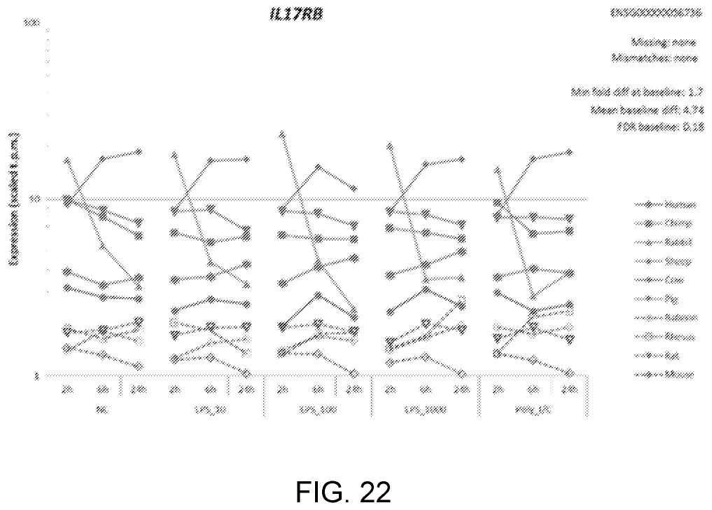

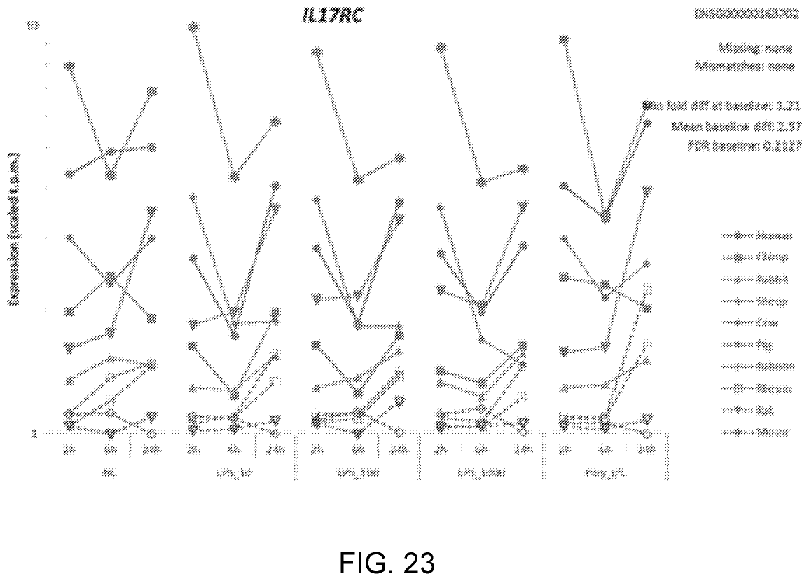

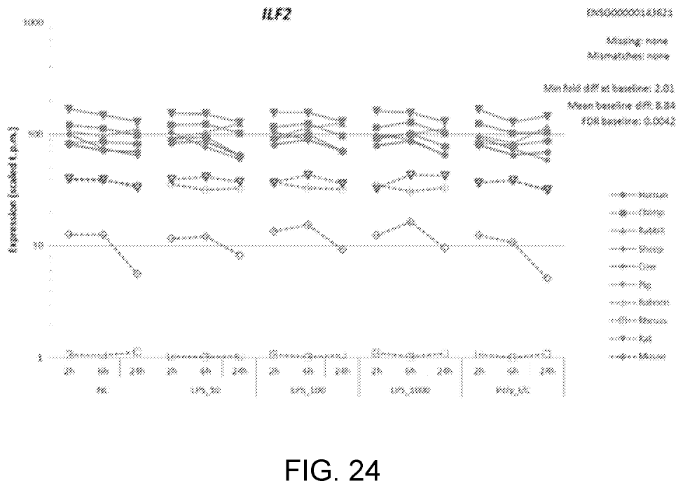

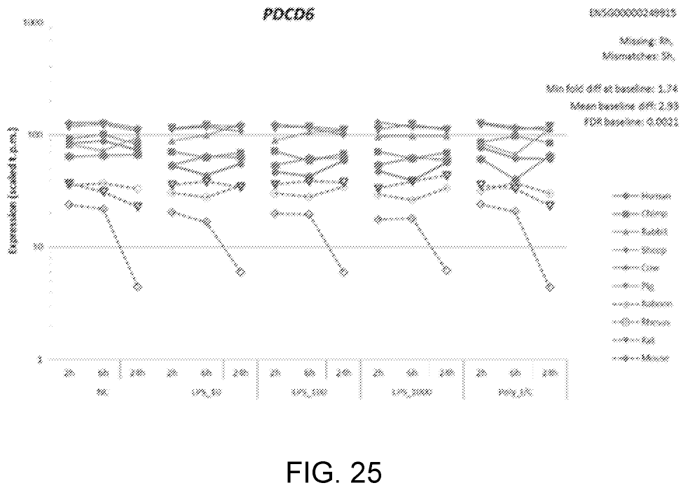

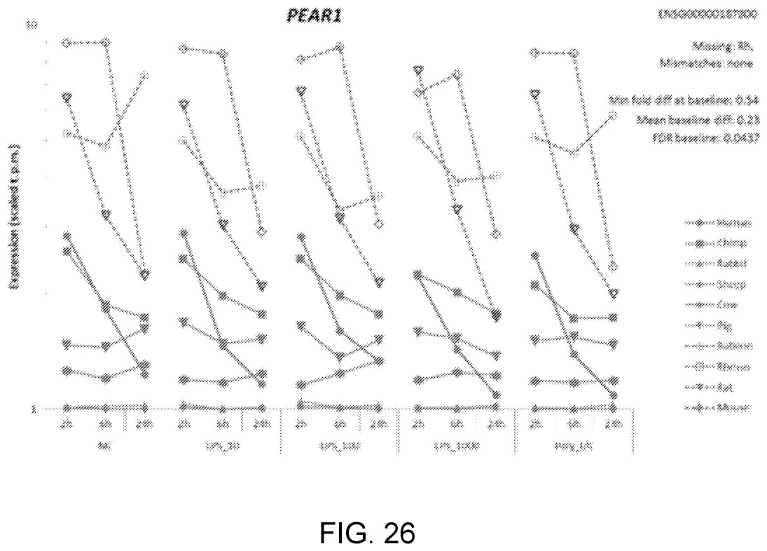

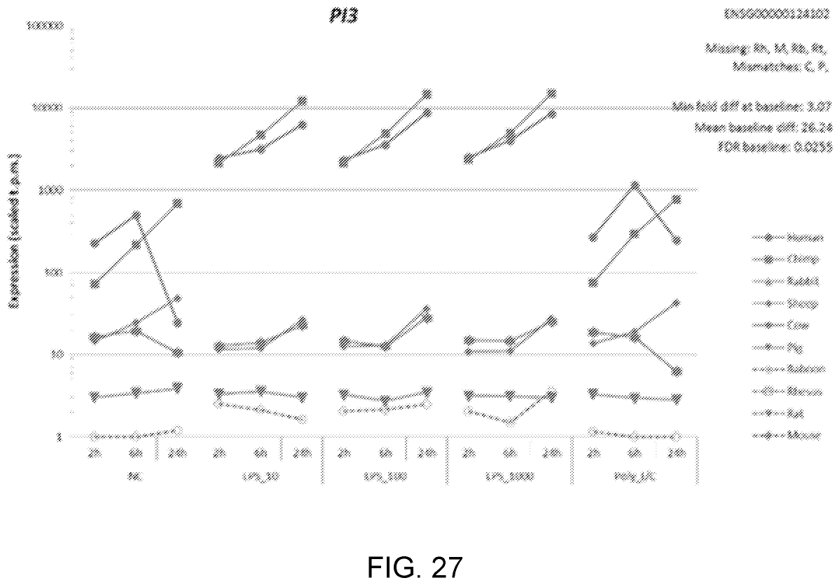

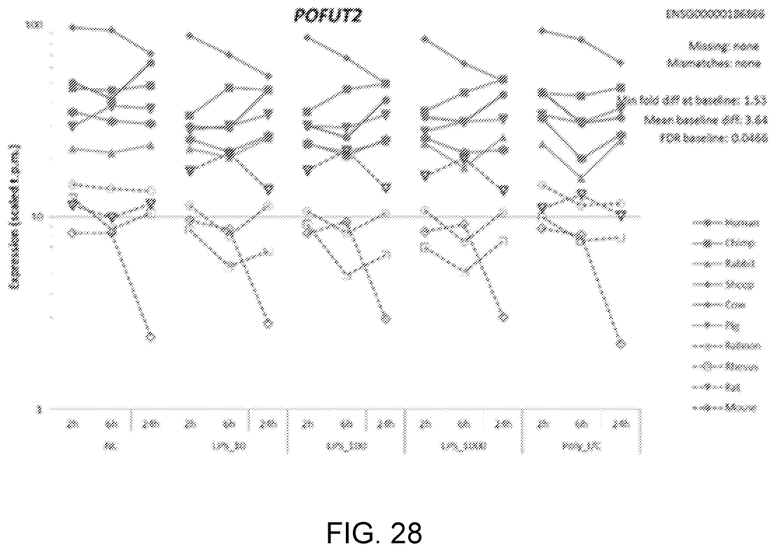

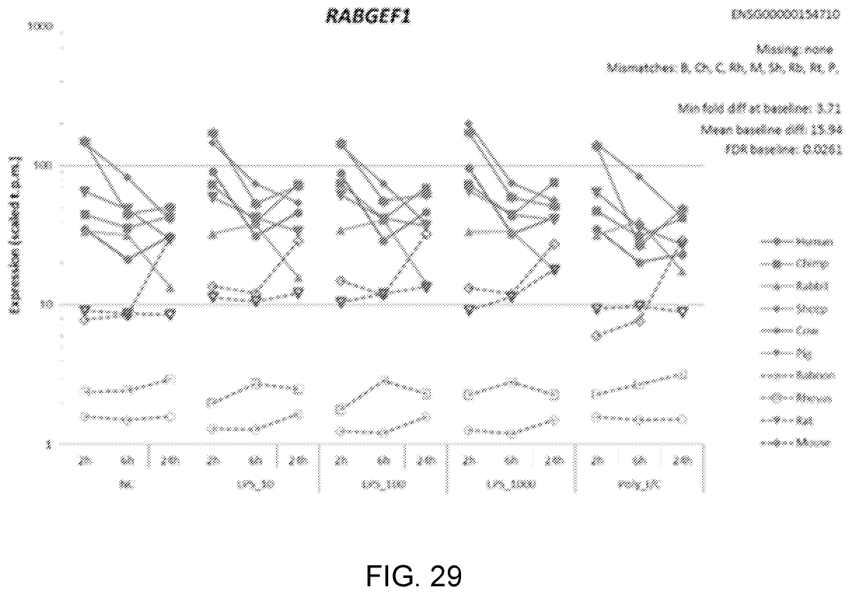

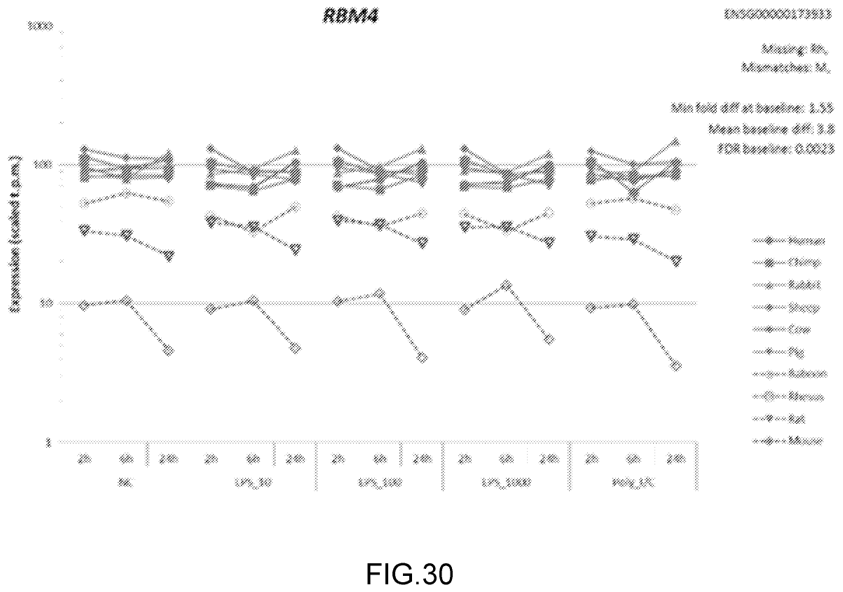

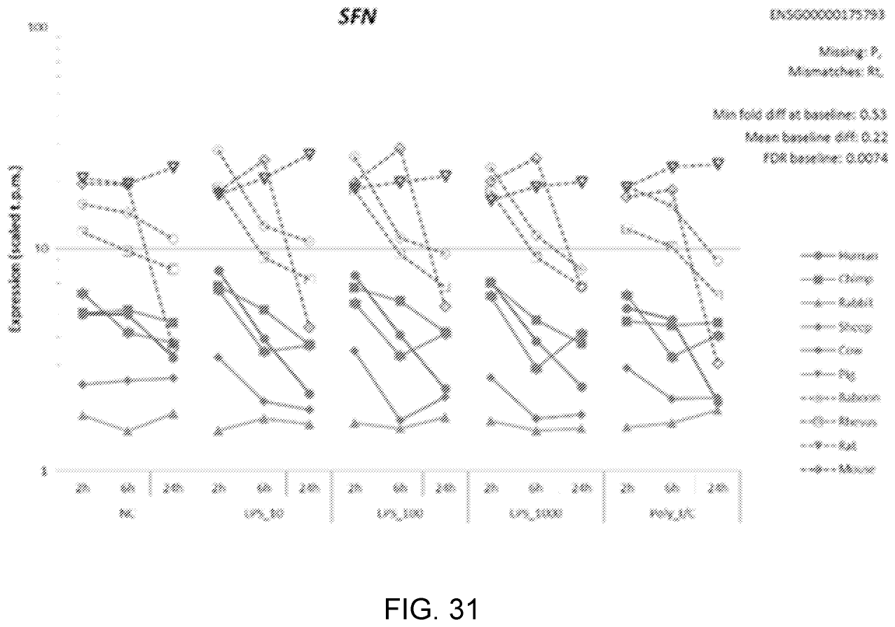

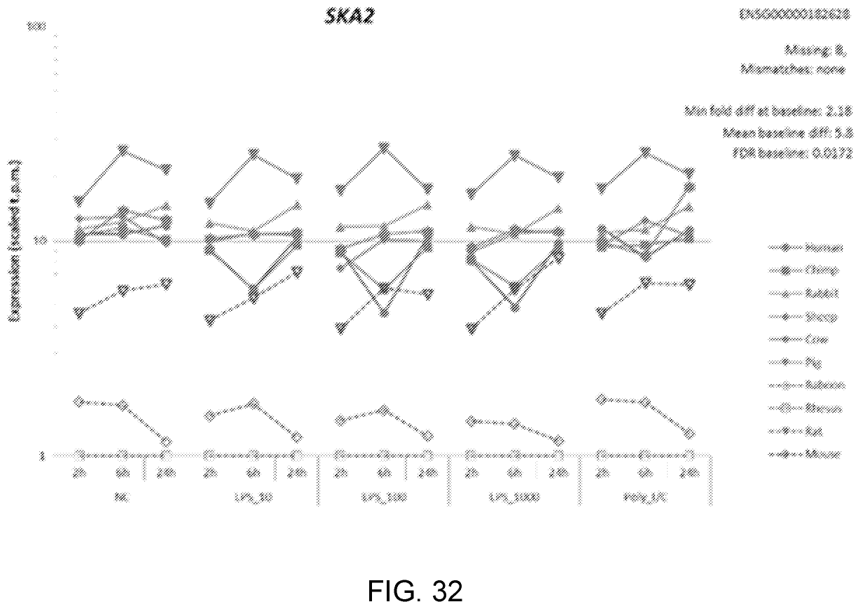

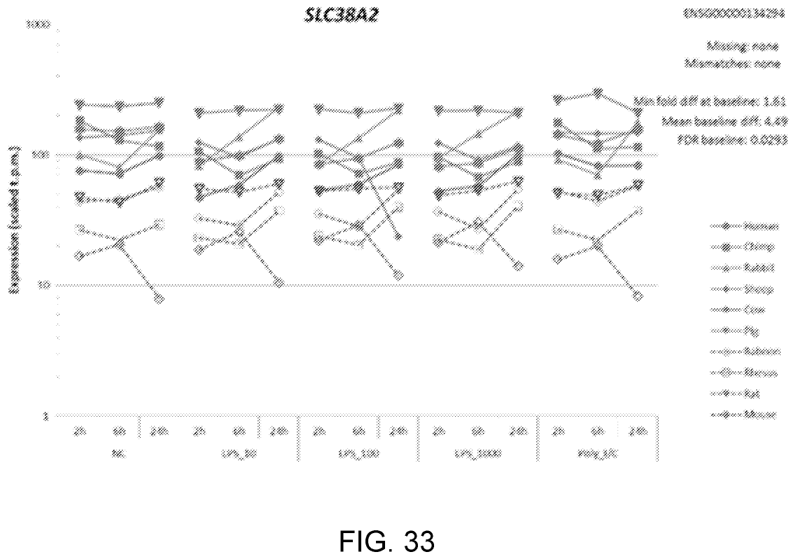

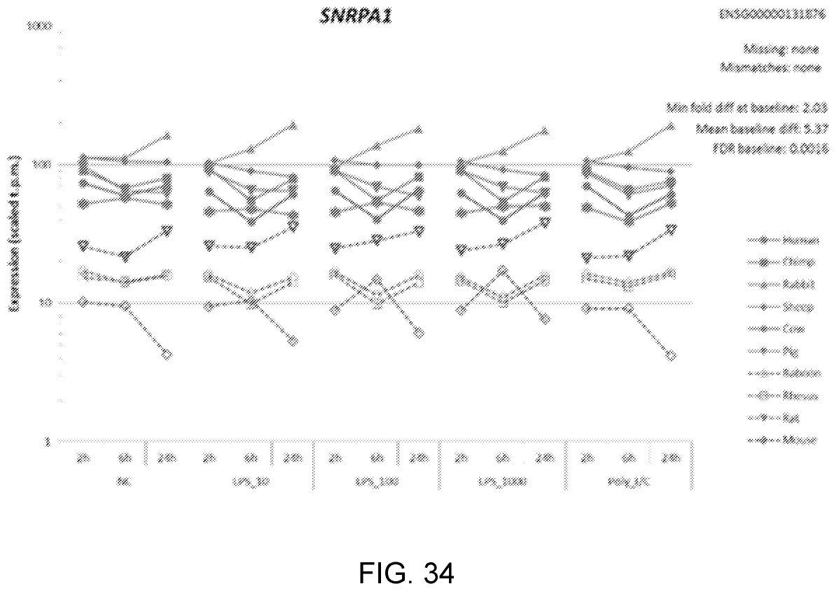

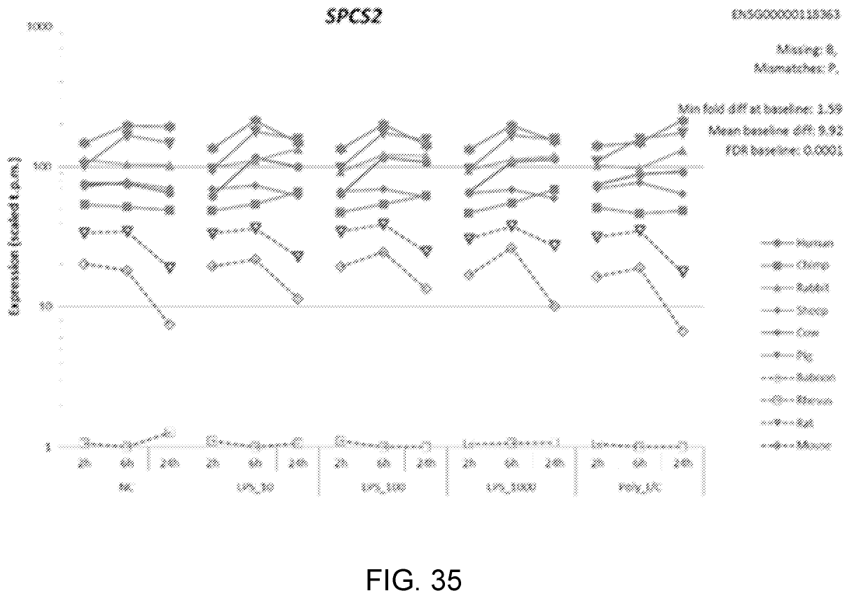

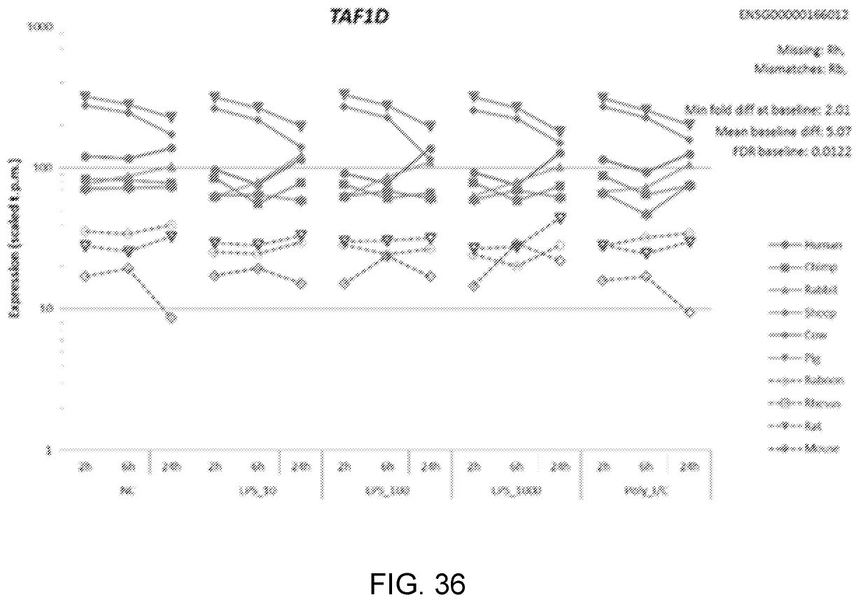

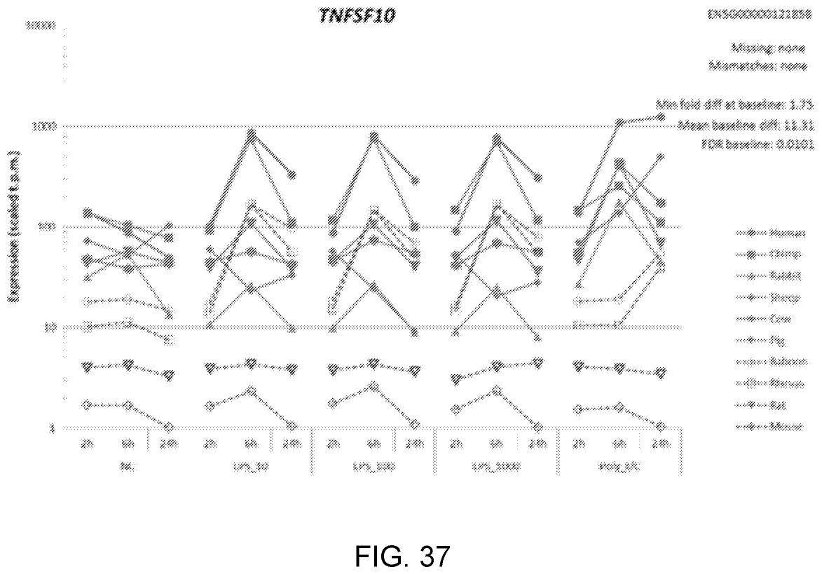

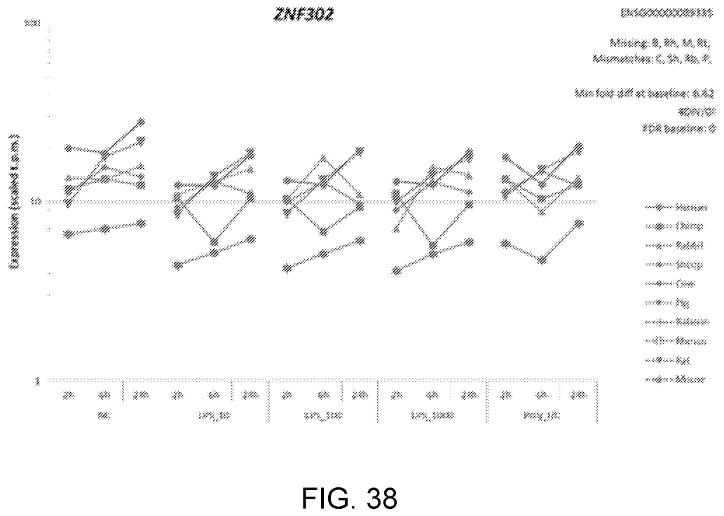

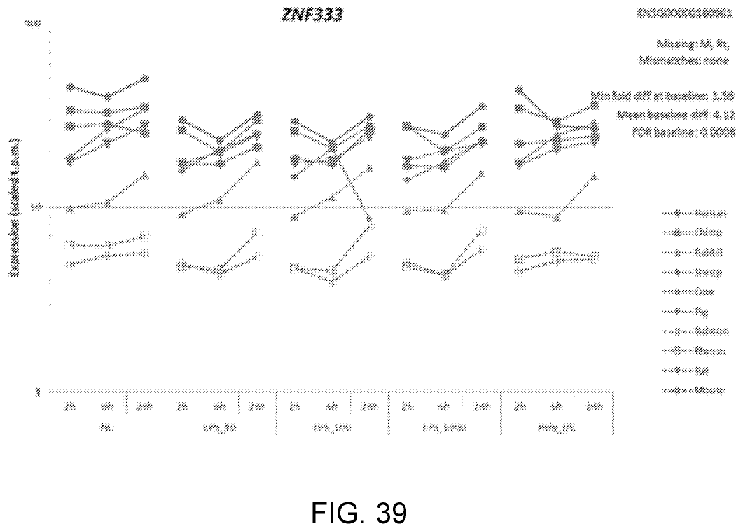

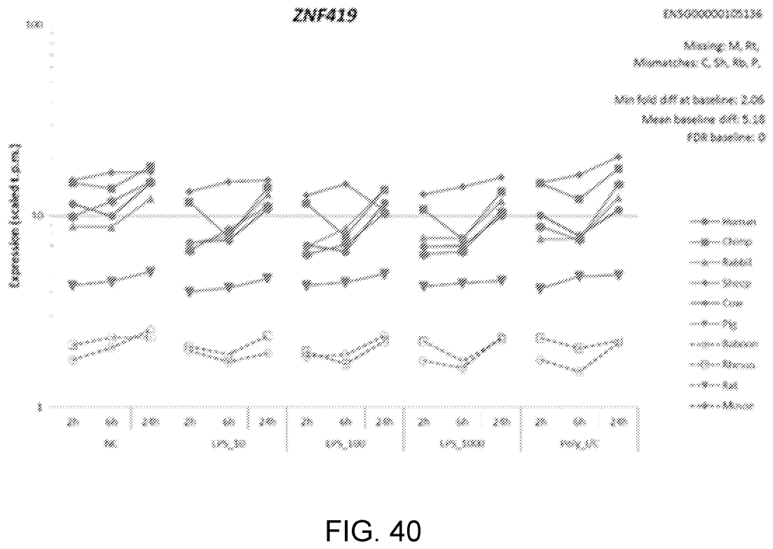

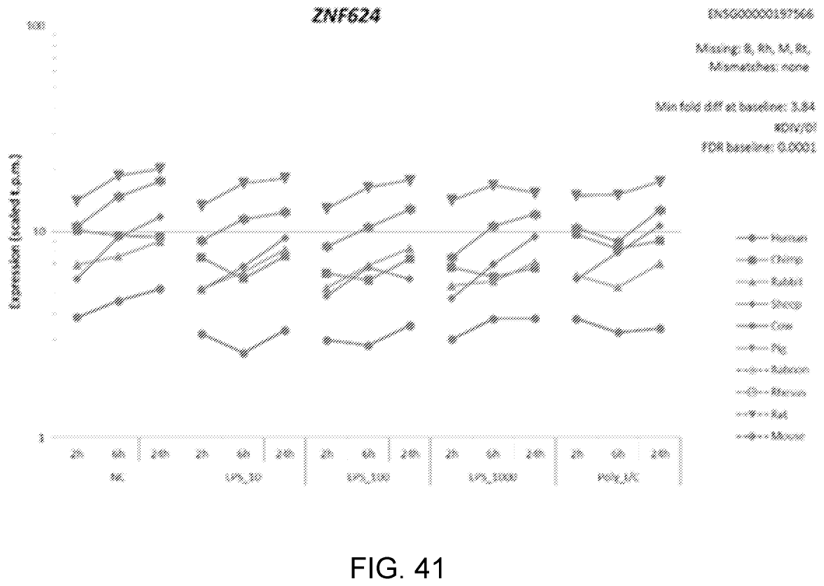

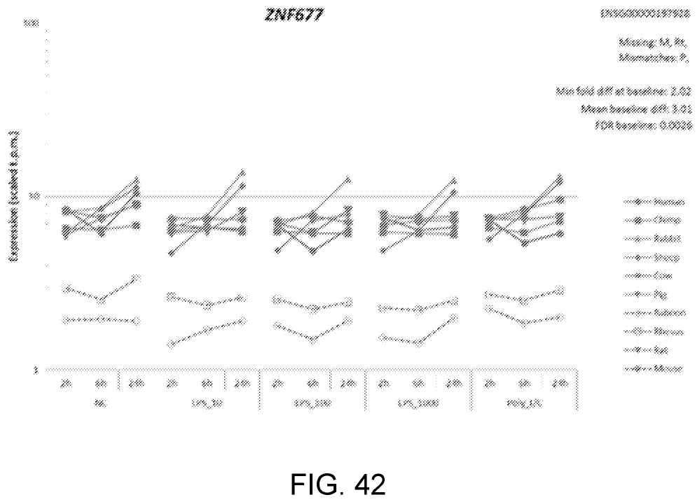

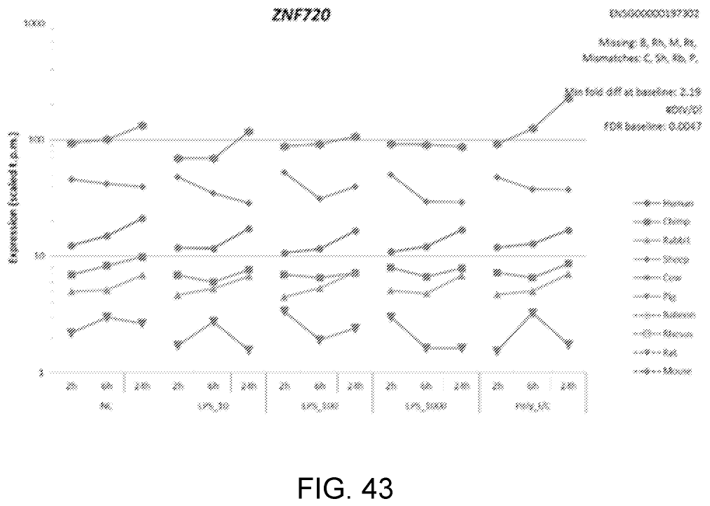

Additional targets which were identified are ARPC4 (FIG. 8), BNIP3 (FIG. 9), CCDC65 (FIG. 10), CENPH (FIG. 11), CHPT1 (FIG. 12), COMMD3 (FIG. 13), DR1 (FIG. 14), EMC9 (FIG. 15), FGD1 (FIG. 16), FIGNL1 (FIG. 17), GGNBP2 (FIG. 18), GNAO1 (FIG. 19), H2AFZ (FIG. 20), HSPB1 (FIG. 21), IL17RB (FIG. 22), IL17RC (FIG. 23), ILF2 (FIG. 24), PDCD6 (FIG. 25), PEAR1 (FIG. 26), PI3 (FIG. 27), POFUT2 (FIG. 28), RABGEF1 (FIG. 29), RBM4 (FIG. 30), SFN (FIG. 31), SKA2 (FIG. 32), SLC38A2 (FIG. 33), SNRPA1 (FIG. 34), SPCS2 (FIG. 35), TAF1D (FIG. 36), TNFSF10 (FIG. 37), ZNF302 (FIG. 38), ZNF333 (FIG. 39), ZNF419 (FIG. 40), ZNF624 (FIG. 41), ZNF677 (FIG. 42), and ZNF720 (FIG. 43).

B. Results from Whole Blood Transcriptome Analysis—Results from FOLD CHANGE Analysis

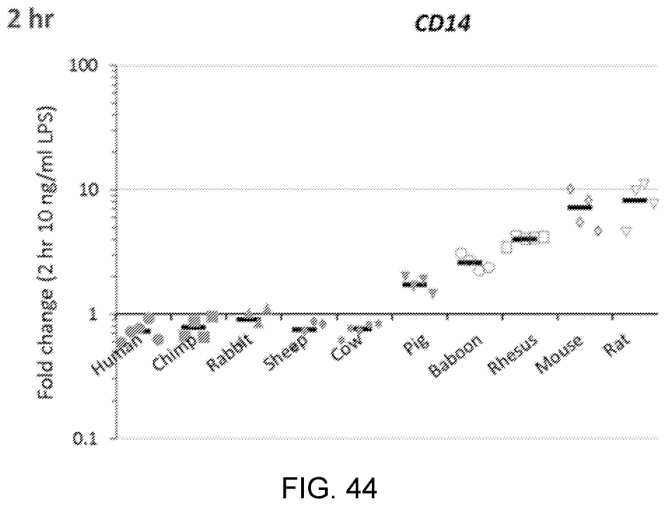

With this analysis, 9 genes responded differently to LPS in sensitive (human, chimp, rabbit, sheep, cow, and pig) vs. resistant species (baboon, rhesus monkey (rhesus macaque), rat, and mouse). The effect of 10 ng/ml LPS on each gene is shown in FIGS. 44-53 for the times when there is significant difference between sensitive and resistant animals. In each case, black bars indicate the mean fold change for each species relative to samples incubated for the same time without LPS. Each data point corresponds to blood from a different animal (or pool of animals, in the case of mice and rats). As previously, sensitive species are noted.

### CD14 and AOAH

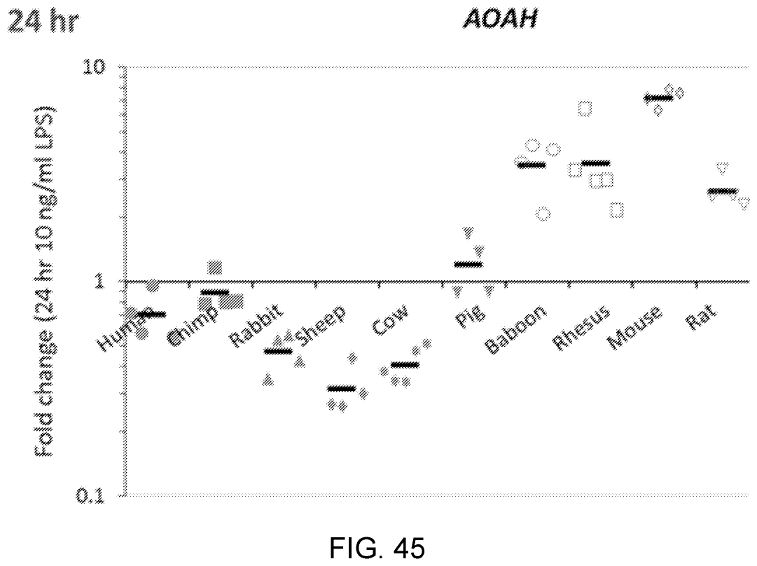

CD14 is known to encode a co-receptor for LPS, and AOAH encodes the principal LPS detoxifying enzyme, acyloxyacyl hydrolase. Referring to FIGS. 44 and 45, the identification of these key proteins involved in LPS metabolism supports our claim for the following 7 gene products.

### SLC8A1

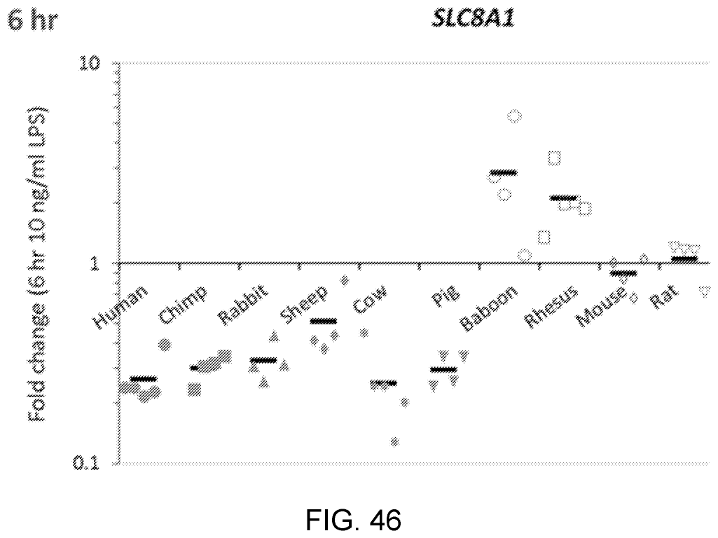

SLC8A1 was identified in our whole blood analysis (FIG. 46). Our methodology is further supported by our finding that benzamil, an inhibitor of Na+/Ca2+ exchangers expected to inhibit the product of SLC8A1, substantially inhibits LPS stimulated TNF secretion in our whole blood assay. These results are presented in Table 5 below. SLC8A1 accordingly was identified in our platform.

TABLE 5

| | % Inhibition of cytokine release, relative to vehicle | | |
|---|---|---|---|
| Benzamil | TNF | IL6 | IL1β |
| 100 μM | 69% ± 6.6% | 65% ± 12.5% | 60% ± 32.8% |
| 50 μM | 23% ± 6.2% | 22% ± 13.2% | 48% ± 33.3% |
| 25 μM | 4% ± 7.9% | 11% ± 9.3% | 32% ± 29.6% |

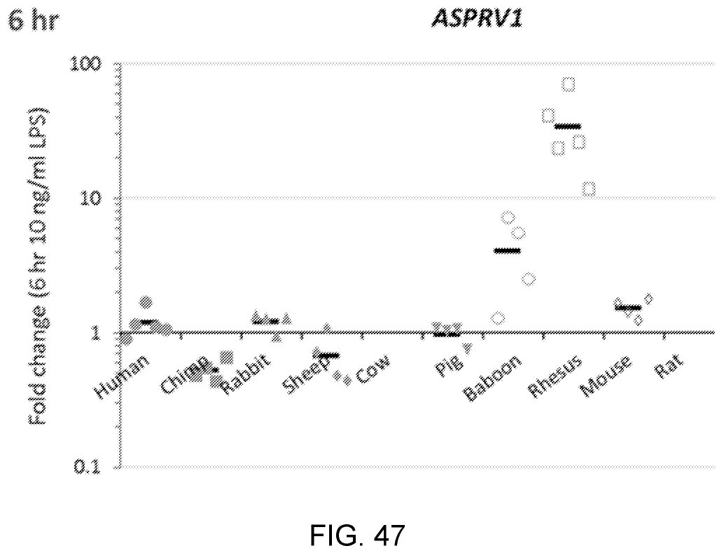

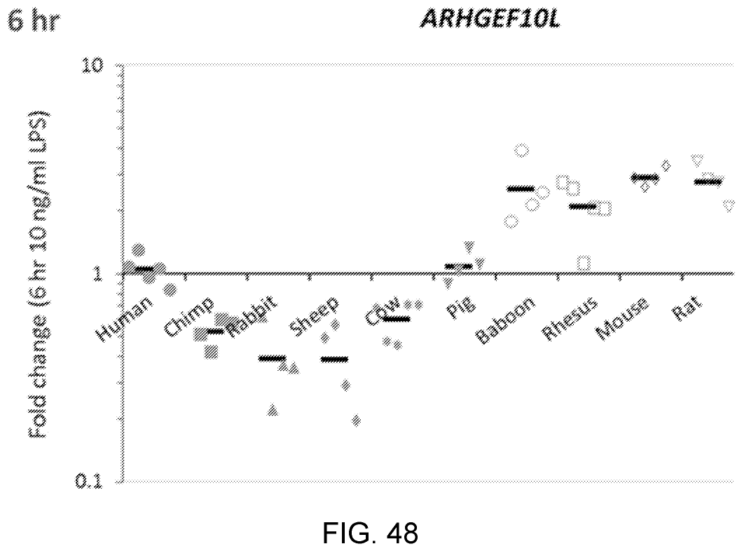

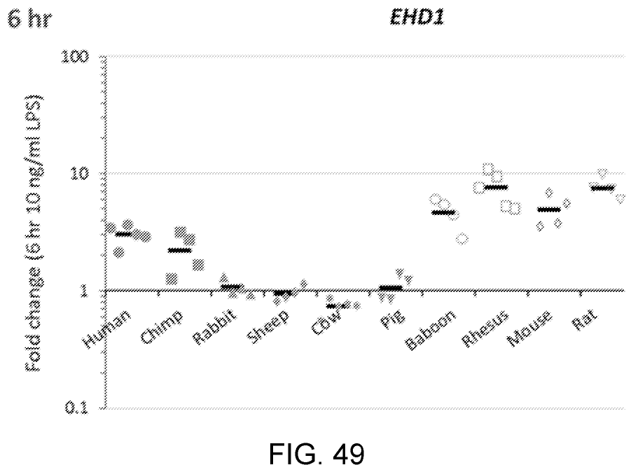

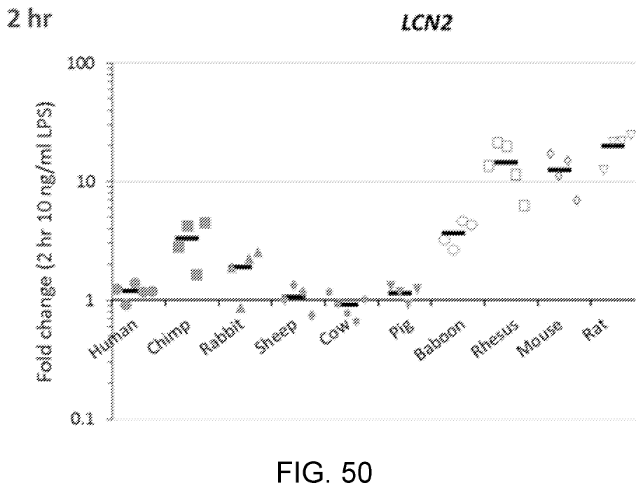

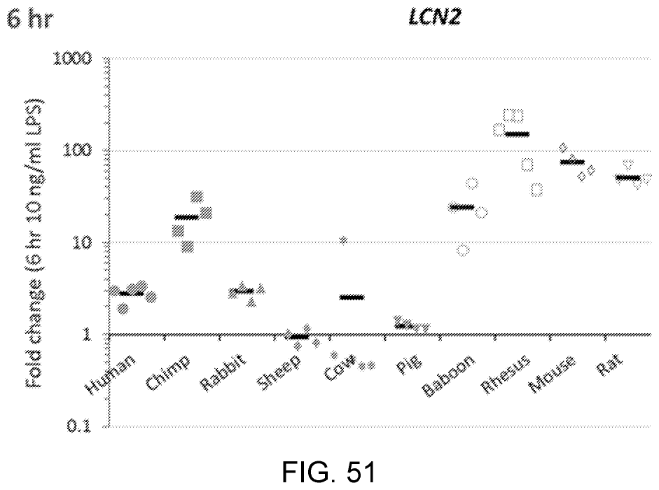

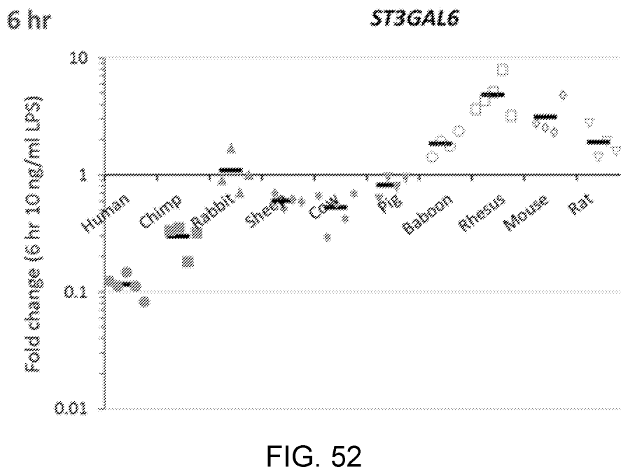

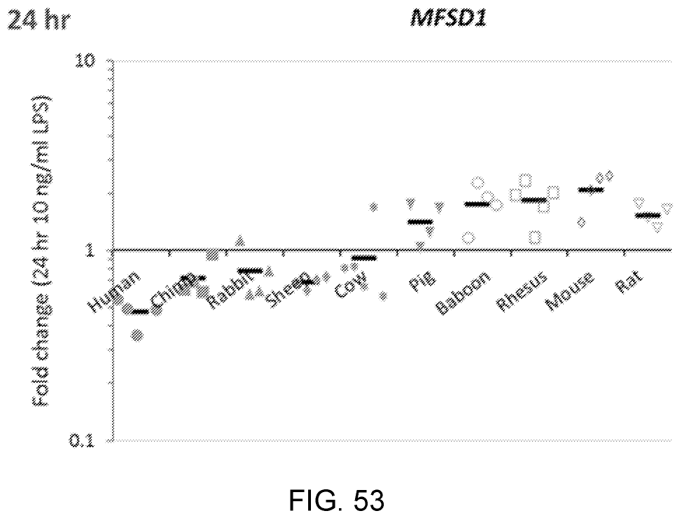

Additional gene targets identified using the FOLD CHANGE analysis are ASPRV1 (FIG. 47), ARHGEF10L (FIG. 48), EHD1 (FIG. 49), LCN2 (FIGS. 50, 51), ST3GAL6 (FIG. 52), and MFSD1 (FIG. 53).

C. Results from Proteomic Analysis

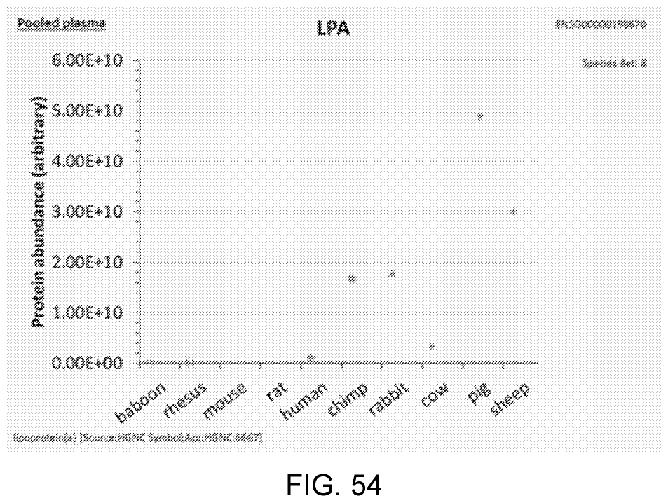

Using this analysis, there were 26 protein targets identified (FIGS. 54 through 79).

The following proteins were part of this group and are provided as examples. In each case, the protein abundance in each species where a given protein was detected is presented as label-free quantitation (LFQ) intensity. As before, resistant species are noted. The corresponding gene name and Ensembl ID is provided, as is the number of species in which the protein was detected.

Lipoprotein (a) (apo(a))

This protein, together with apolipoprotein B, cholesterol, and other lipids, comprises Lp(a)—a low-density lipoprotein particle. Elevated serum Lp(a) is recognized as a risk factor for cardiovascular disease. Both Lp(a) and apo(a) itself are established therapeutic targets for cardiovascular disease. In this context, Lp(a) is believed to promote inflammation by mediating deposition of cholesterol on the arterial endothelium. However, in addition to well-established roles promoting inflammation, Lp(a) may exert anti-inflammatory effects by mediating clearance of both oxidized phospholipids and LPS, and reduced Lp(a) has been reported during late onset neonatal sepsis, indicating a more complicated role in different inflammatory diseases. Our identification of a difference in apo(a) protein abundance expression between plasma from sensitive and resistant species serves as a validation of the disclosed methodology (FIG. 54).

### Adhesion Molecules

Both endothelial cell selective adhesion molecule (ESAM) and platelet and endothelial cell adhesion molecule (PECAM1) are immunoglobulin-type proteins involved in regulating barrier endothelial integrity and leukocyte transmigration. Interpretation of molecular and knockout studies is often confounded by the existence of both transmembrane, signal transducing forms and secreted, serum forms that have the potential to act as decoy inhibitors. However, both are known to act in not only chronic but also acute inflammatory disorders, including by regulation migration of pro-inflammatory leukocytes into the tissues. Consistent with the reduced expression we detect in serum of resistant species, Pecam1 knockout mice show enhanced mortality in the LPS challenge model of sepsis.

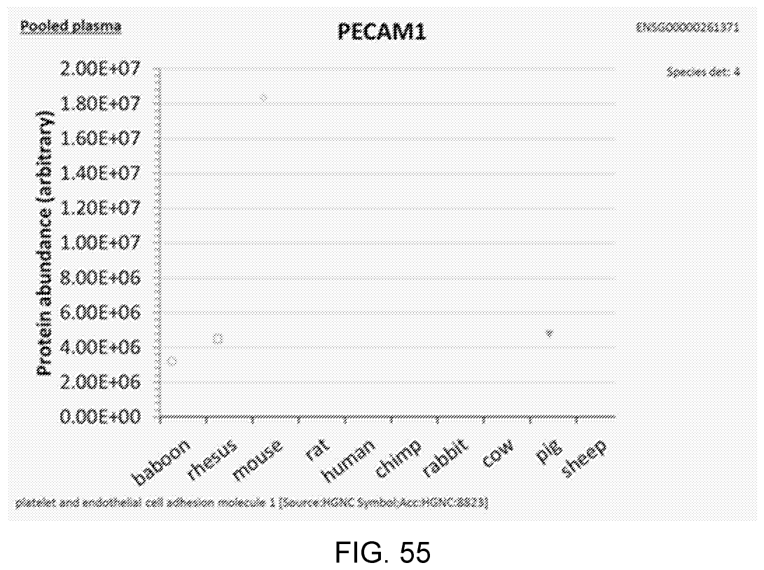

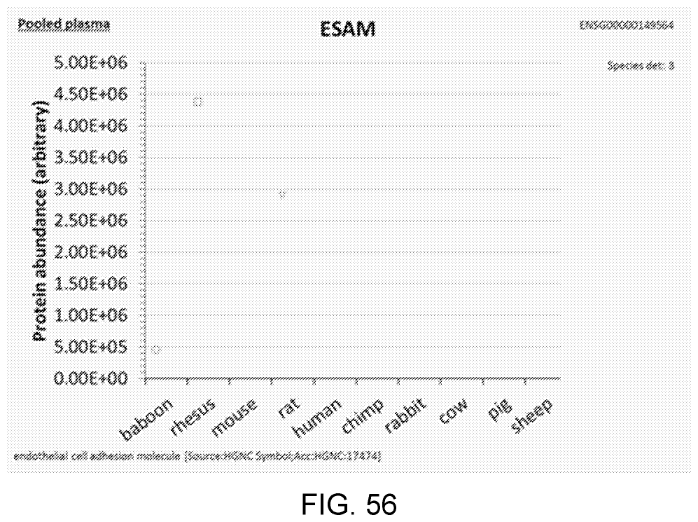

In humans, both sPECAM1 and sESAM have been investigated as biomarkers for sepsis, cardiovascular disease, and other inflammatory diseases. The identification of these known modulators of inflammation in our differential plasma proteomics study validates the disclosed method (FIGS. 55 and 56). Their importance in regulating endothelial permeability reinforces the link between endothelial permeability, inflammation, and serum composition.

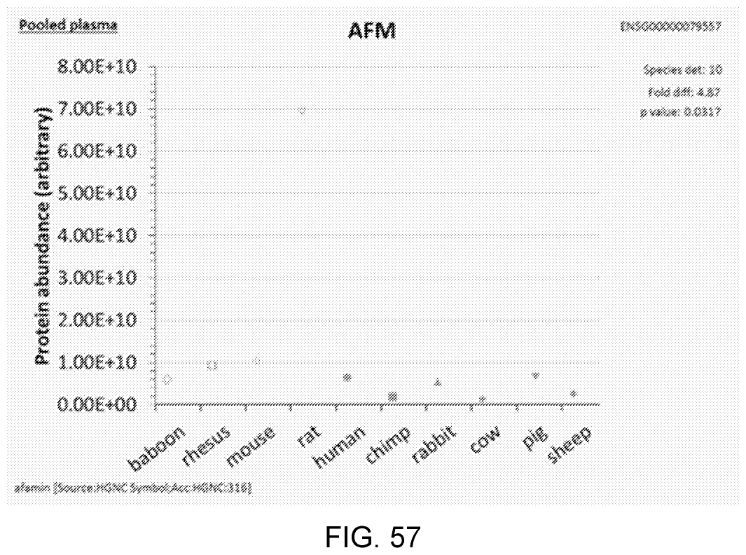

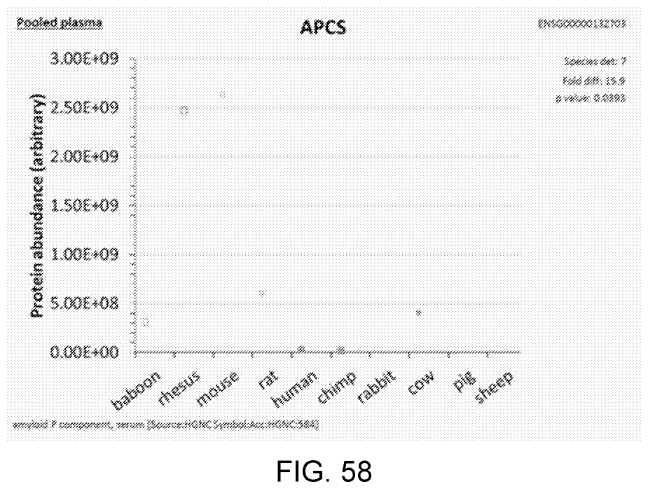

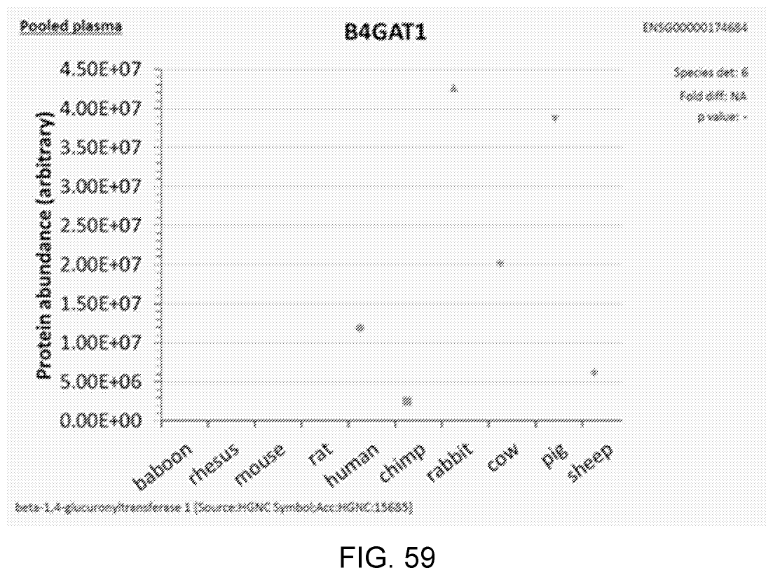

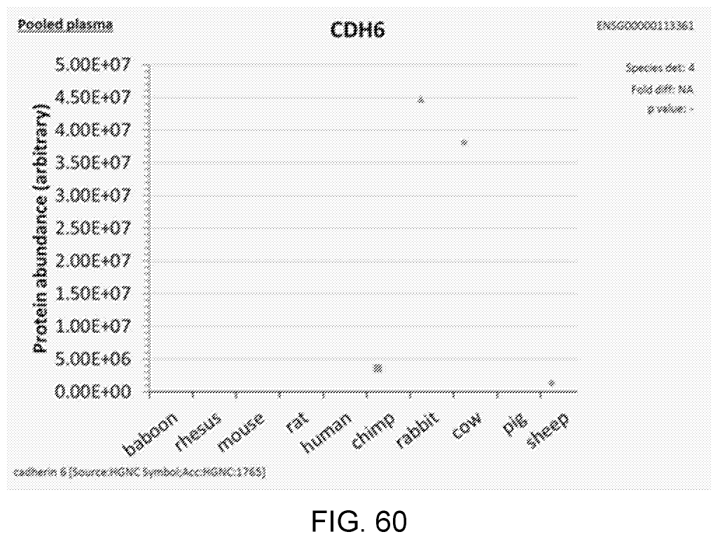

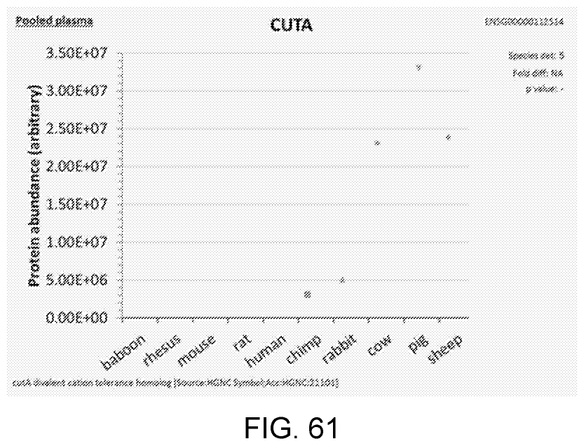

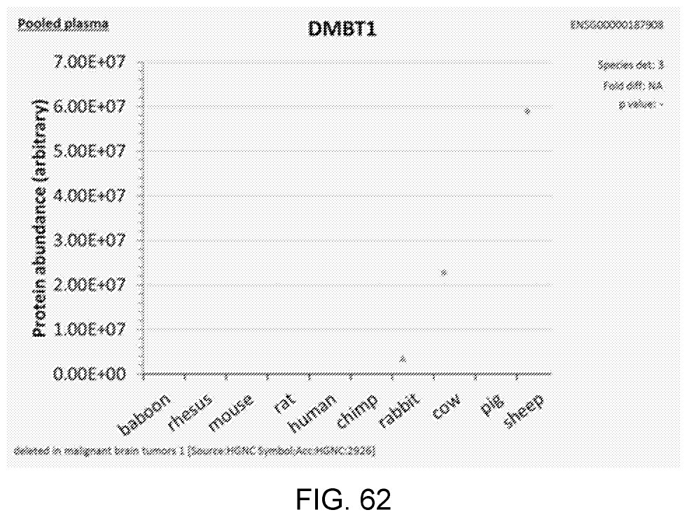

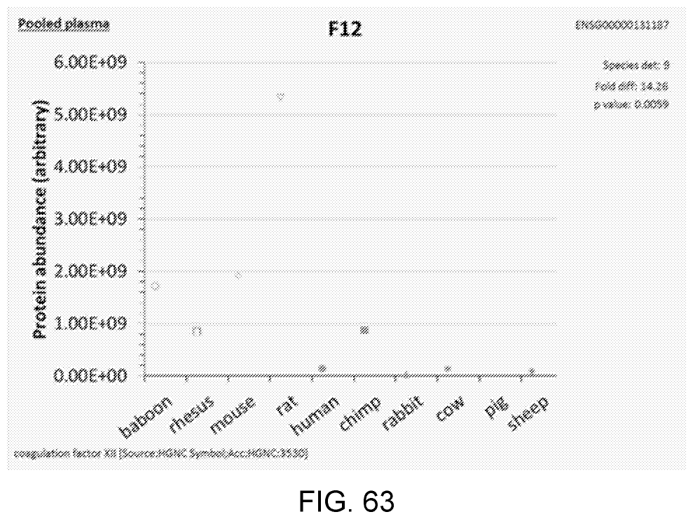

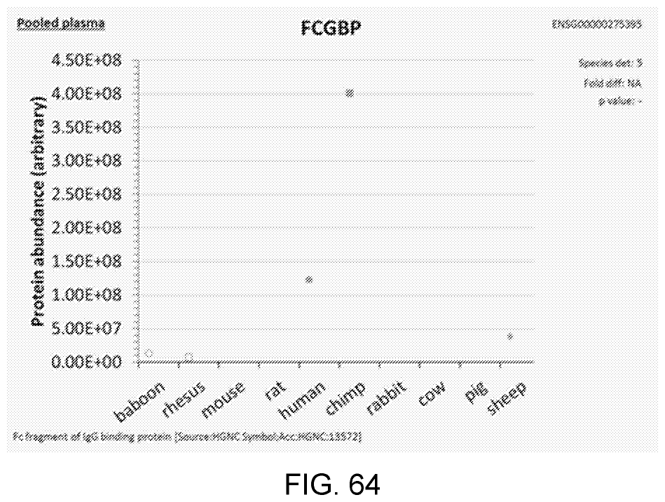

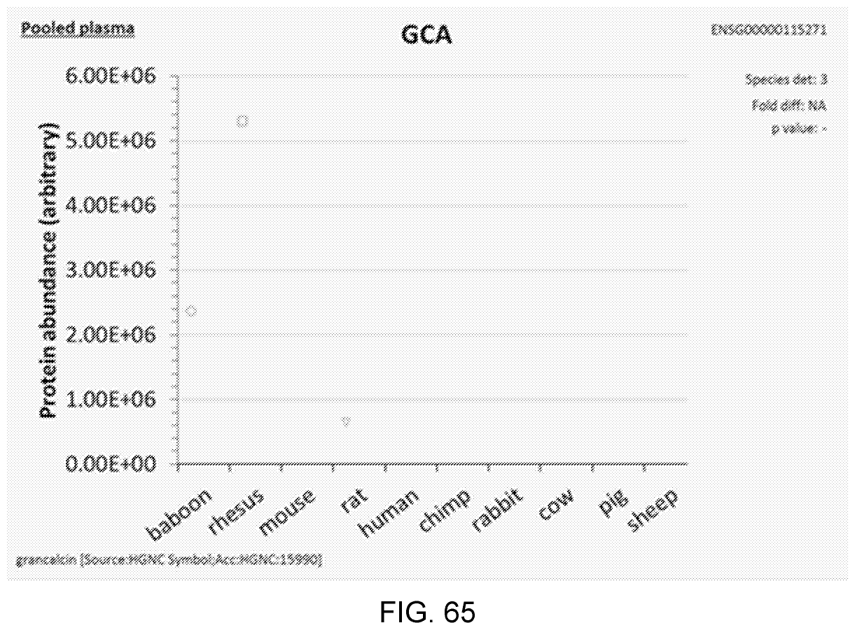

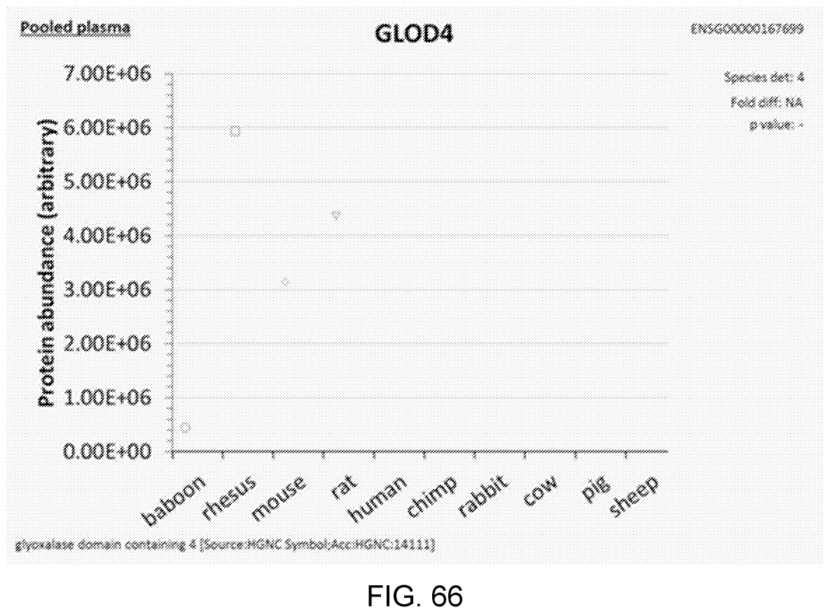

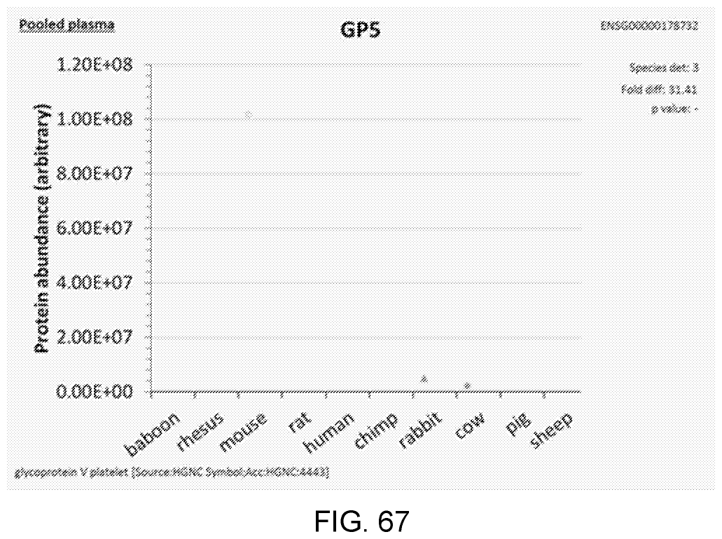

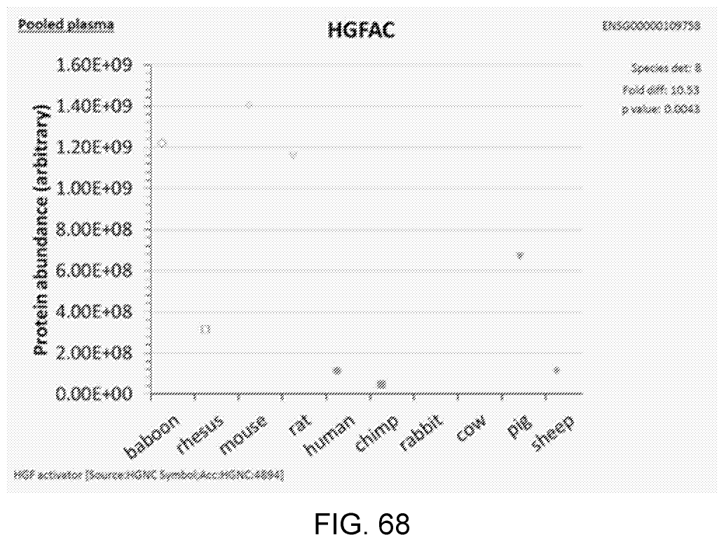

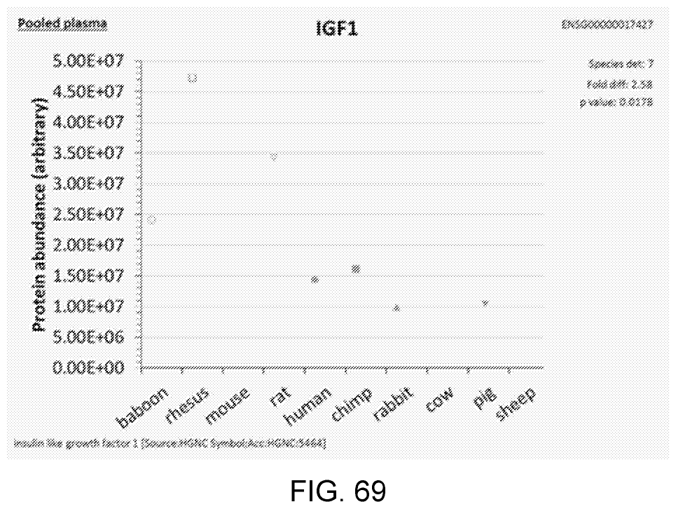

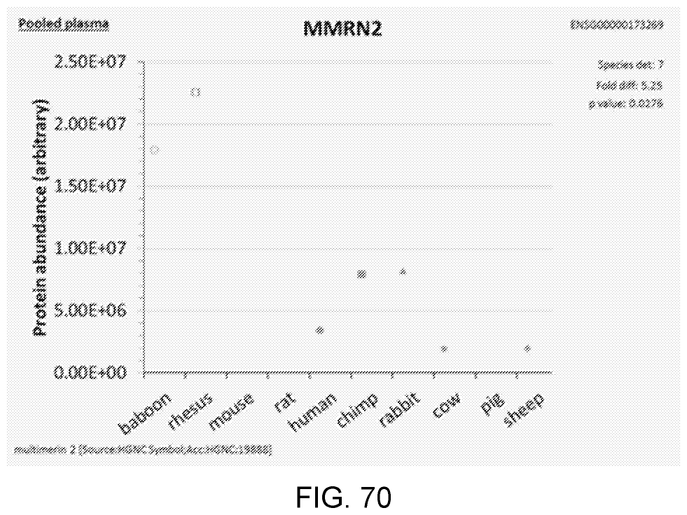

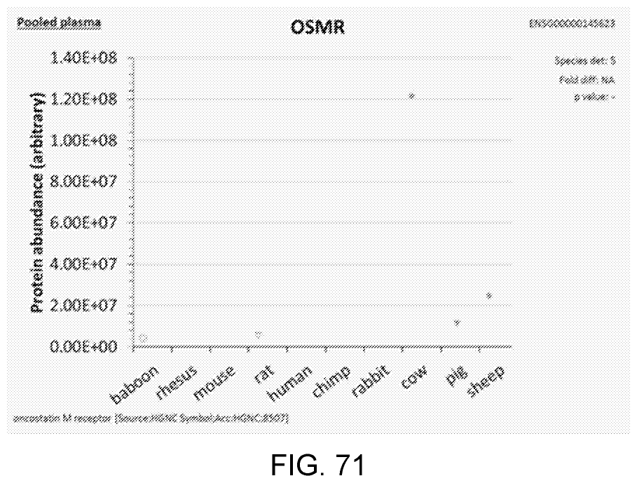

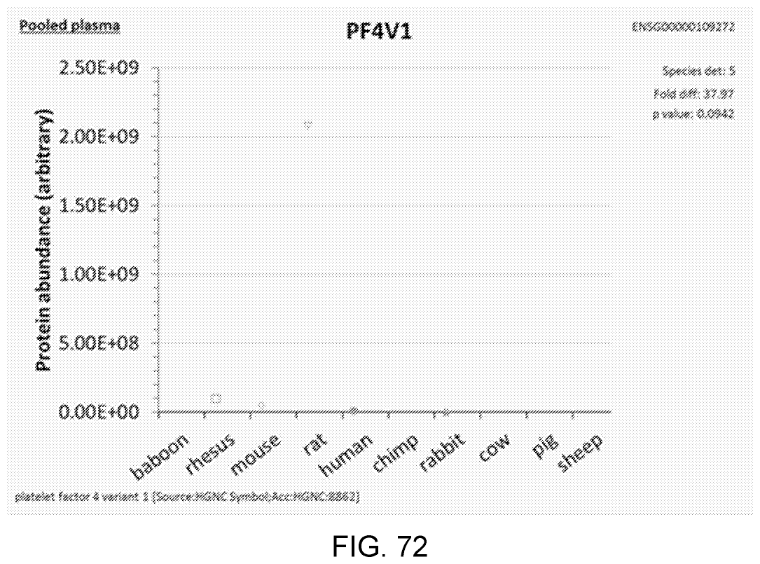

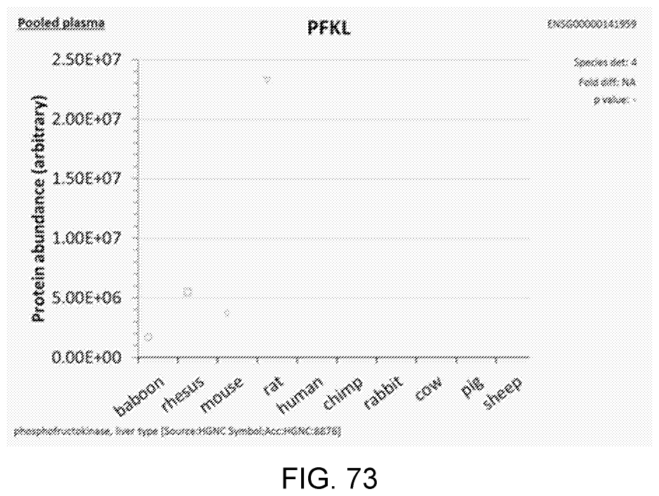

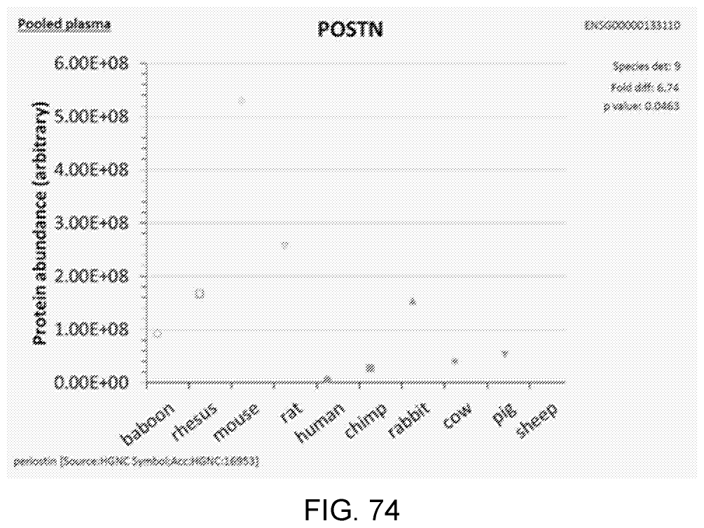

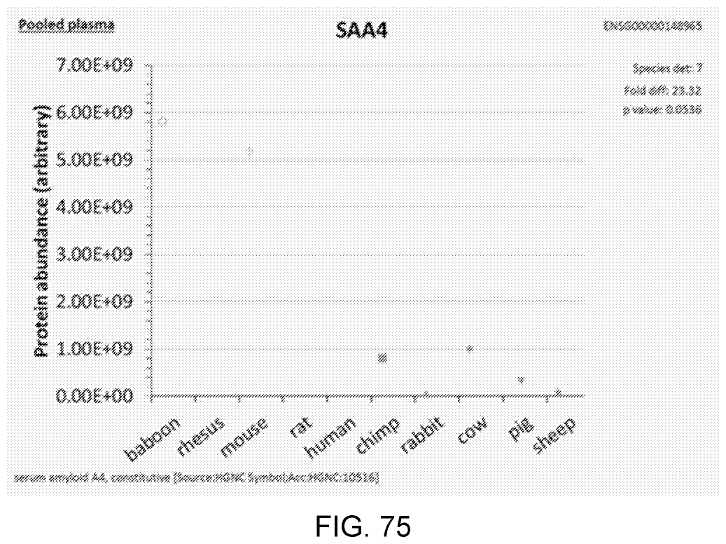

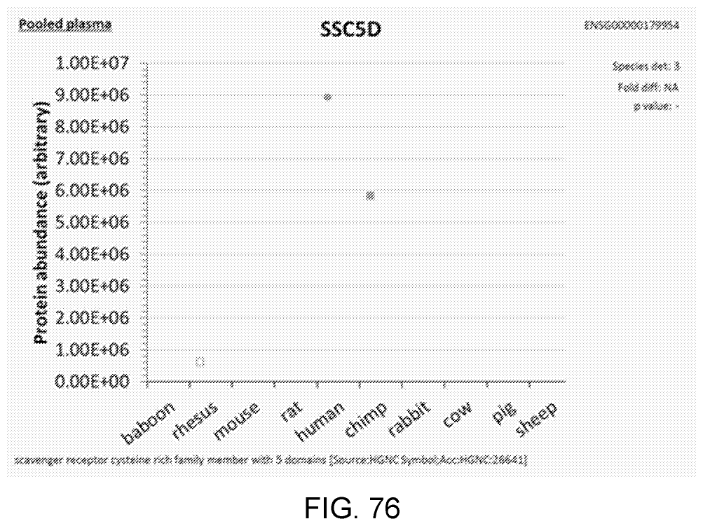

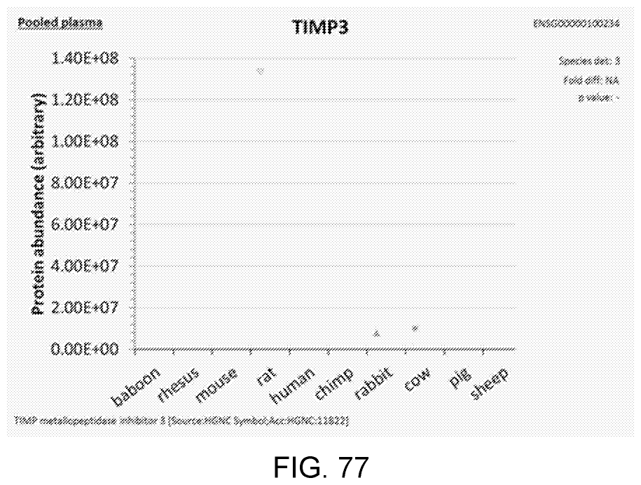

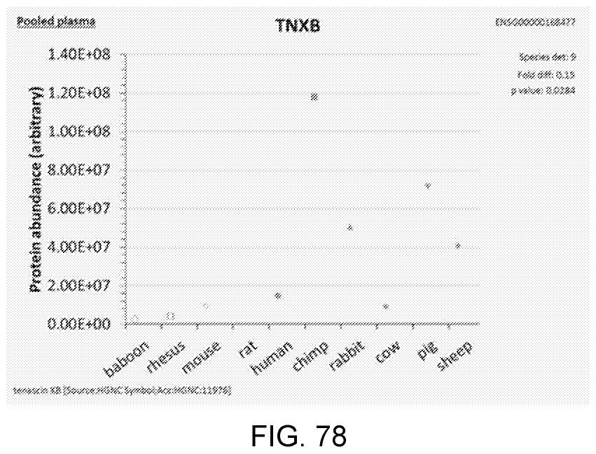

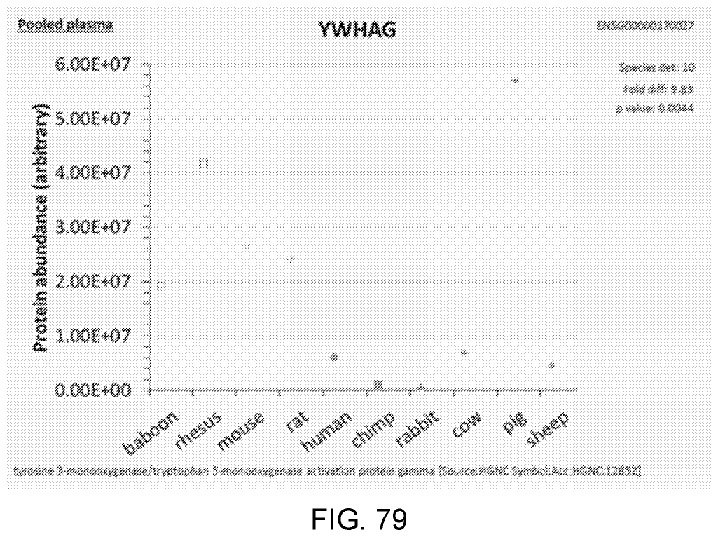

Additional identified targets using this methodology include Afamin (AFM) (FIG. 57), Amyloid P component, serum (APCS) (FIG. 58), Beta-1,4-glucuronyltransferase 1 (B4GAT1) (FIG. 59), Cadherin 6, type 2, K-cadherin (CDH6) (FIG. 60), CutA divalent cation tolerance homolog (E. coli) (CUTA) (FIG. 61), Deleted in malignant brain tumors 1 (DMBT1)(FIG. 62), Coagulation factor XII (Hageman factor) (F12) (FIG. 63), Fc fragment of IgG binding protein (FCGBP) (FIG. 64), Grancalcin (GCA) (FIG. 65), Glyoxalase domain containing 4 (GLOD4)(FIG. 66), Glycoprotein V (platelet) (GP5) (FIG. 67), HGF activator (HGFAC) (FIG. 68), Insulin like growth factor 1 (IGF1) (FIG. 69), Multimerin 2 (MMRN2) (FIG. 70), Oncostatin M receptor (OSMR) (FIG. 71), Platelet factor 4 variant 1 (PF4V1) (FIG. 72), Phosphofructokinase, liver (PFKL) (FIG. 73), Periostin, osteoblast specific factor (POSTN) (FIG. 74), Serum amyloid A4 (SAA4)(FIG. 75), Scavenger receptor cysteine rich family, 5 domains (SSC5D) (FIG. 76), TIMP metallopeptidase inhibitor 3 (TIMP3) (FIG. 77), Tenascin XB (TNXB) (FIG. 78), and Tyrosine 3-monooxygenase/tryptophan 5-monooxygenase activation protein, gamma (YWHAG) (FIG. 79).

### C. Results for Endothelial Analysis

Using this analysis, 38 endothelial genes were identified as targets. These include known mediators of inflammation and endothelial barrier integrity. This evidence provides further validation for our model.

Given the involvement of both endothelial barrier permeability and leukocyte cytokine secretion in the inflammatory process, we tested chemical modulators of some of the identified targets in both the ECIS permeability assay and the whole blood cytokine release assay. Gene expression plots are shown for the two or three RNAseq experiments that showed a difference between sensitive and resistant species. As before, the sensitive condition (species of cell, species of serum, or human cells without HKEC pretreatment) is shown and the resistant condition are noted. Also provided, where appropriate, are the ECIS electrical conductance plots of human lung microvascular endothelial cells (LMVECs) following treatment with specific small molecules and heat killed E. coli (HKEC), and plots of cytokine release from human whole blood following LPS stimulation in the presence of specific drugs.

Examples that provide validation of the method and/or the specific target are shown first.

### DPP4

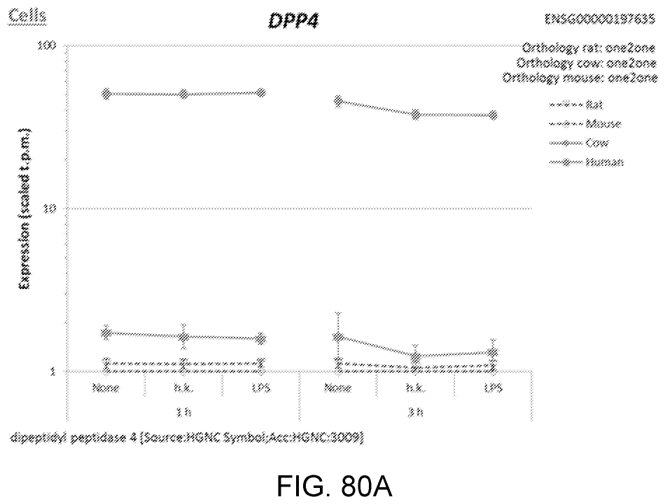

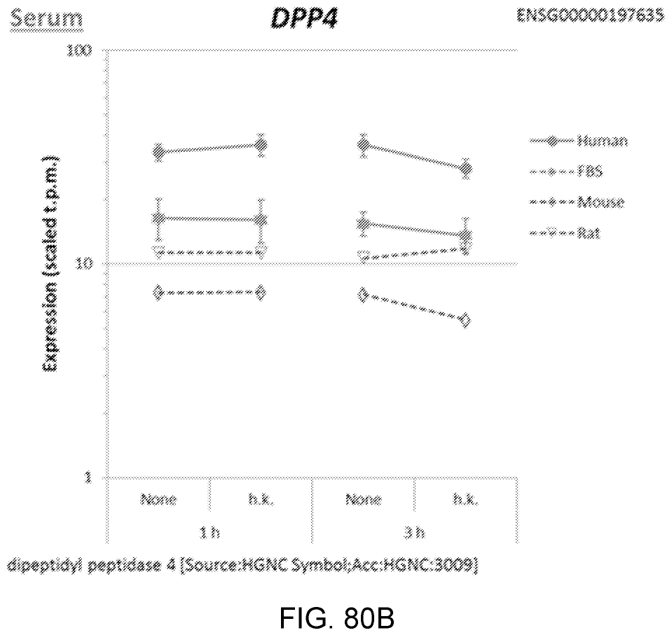

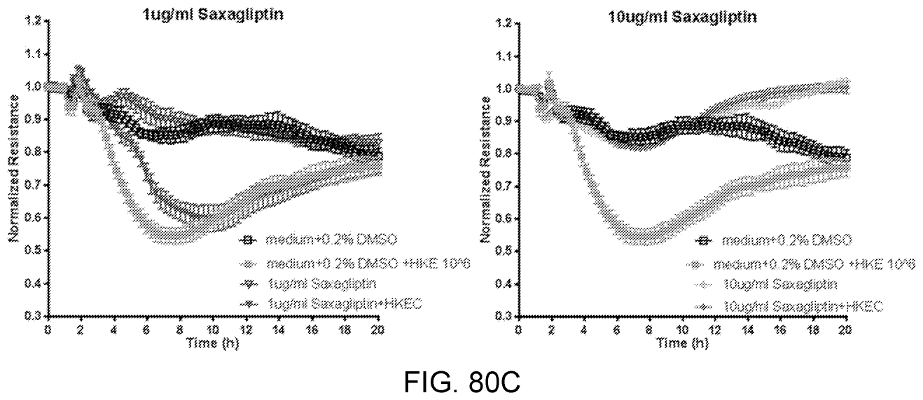

DPP4 encodes dipeptidyl peptidase 4, an established target in type 2 diabetes mellitus that may also be involved in other metabolic diseases. DPP4 inhibitors have been proposed to ameliorate inflammation by mechanisms including inflammasome and TLR4/p38 inhibition. This is consistent with the greater expression we observed in sensitive endothelial cells, especially those from humans relative to rat and mouse. While linked to vascular endothelial function, especially in the context of cardiovascular disease, DPP4 has not been convincingly linked to endothelial permeability during inflammation or other stresses. FIGS. 80A and 80B show DPP4 expression in endothelial cells and in endothelial cells cultured in serum, respectively.

Further, our results in FIG. 80C show that the DPP4 inhibitor, saxagliptin, causes a dose dependent reduction in human LMVEC barrier permeability following HKEC stimulation. These data validate our method for target discovery.

### BMP4

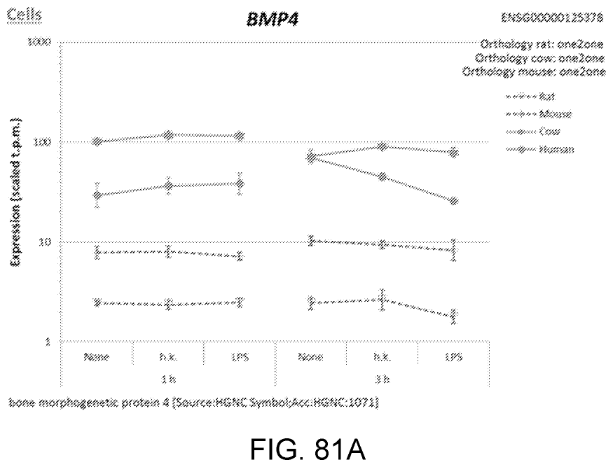

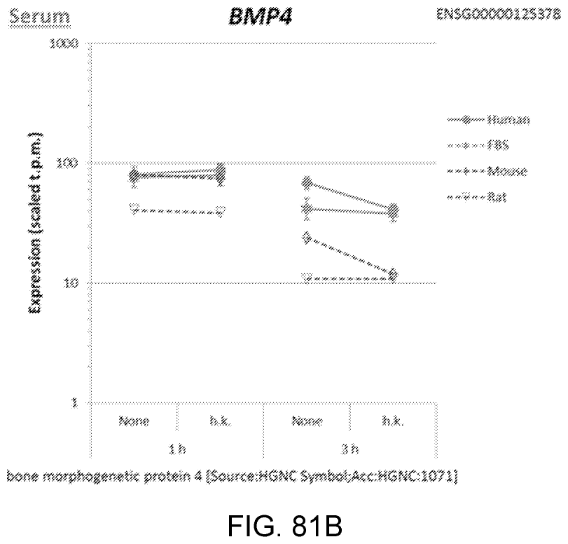

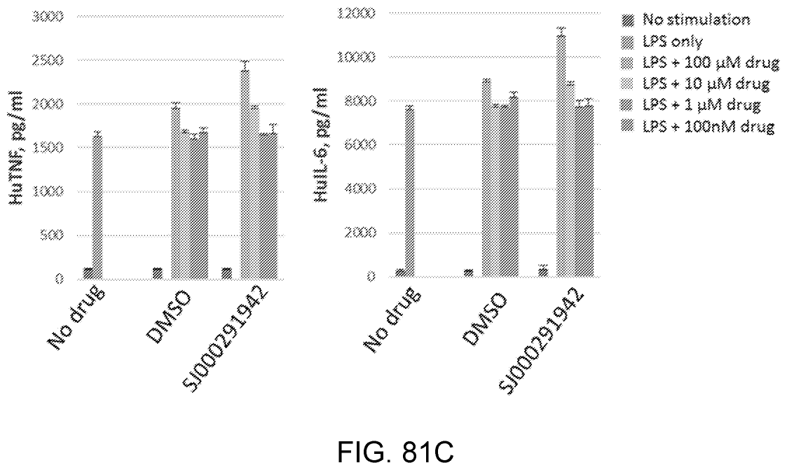

BMP4 is expressed more highly in human endothelial cells grown in serum from sensitive animals and in endothelial cells from sensitive animals (FIGS. 81A and 81B). The gene product, bone morphogenic family 4, is a TGFβ family member with pleiotropic roles in development. This includes endothelial development, where there is good evidence that it promotes barrier permeability. Endothelial-specific gene knockout reduces leukocyte infiltration in thioglycolate elicited peritoneal inflammation model. Furthermore, BMP4 exacerbates cardiovascular disease in the ApoE4 mouse model by mediating inflammation and vascular permeability. Repression of BMP4 expression may contribute to the protective effect of mevastatin in cardiovascular disease.

We have found that an agonist of BMP receptor signaling, SJ000291942, enhances proinflammatory cytokine secretion in our whole blood model (FIG. 81C). Thus, our transcriptomic analysis shows higher expression of BMP4 in sensitive cells, correctly identifying a factor that connects inflammation and endothelial permeability.

### MYLK2 and MYLK4

MYLK2 and MYLK4 encode myosin light chain kinases (MLCKs). Phosphorylation of myosin by MLCKs causes contraction in a range of contractile cells, including endothelial cells, where contraction leads to barrier hyperpermeability. Transient transfection of activated MLCK protein has been shown to increase albumin permeability of a bovine coronary vein endothelial cell monolayer in vitro.

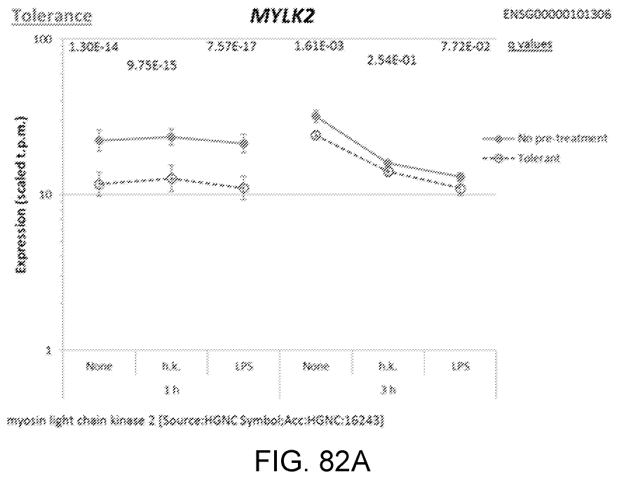

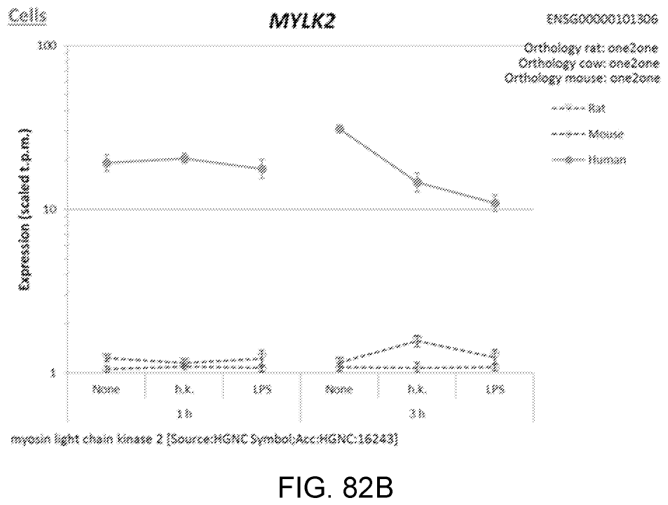

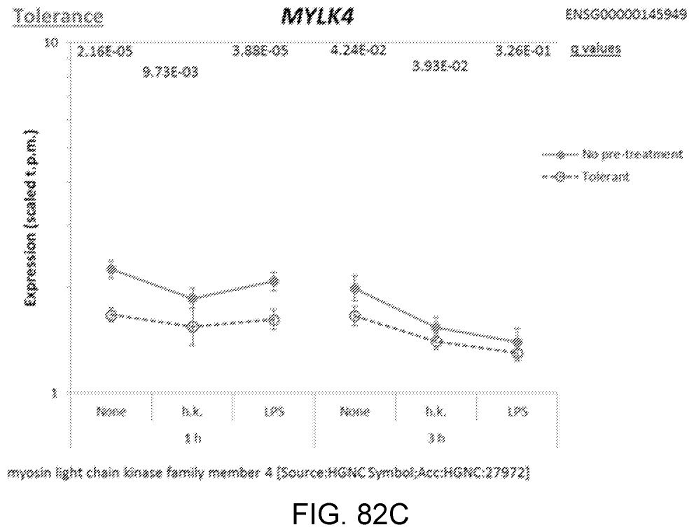

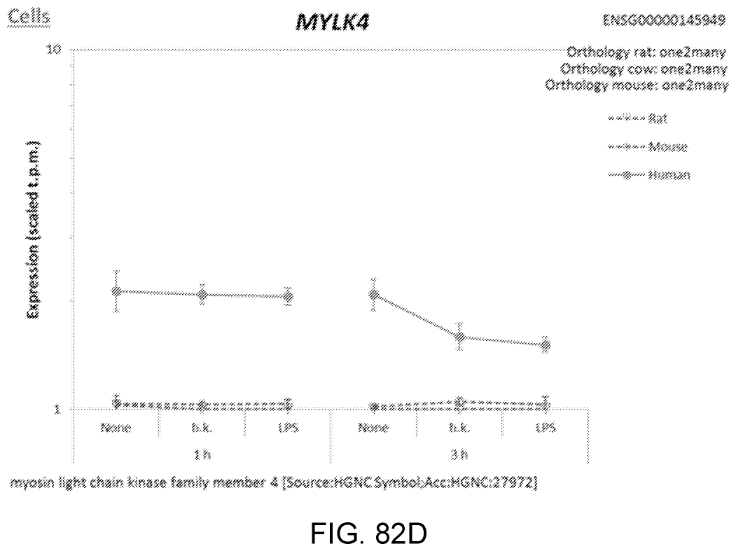

Moreover, chemical MLCK inhibition reduces barrier hyperpermeability in response to treatment of renal endothelial cells with TNF. Thus, the greater expression of two MLCK genes in human LMVECs relative to tolerant or rodent cells is consistent with their greater propensity for hyperpermeability and supports the validity of our approach to identify novel targets for intervention (FIGS. 82A-82D).

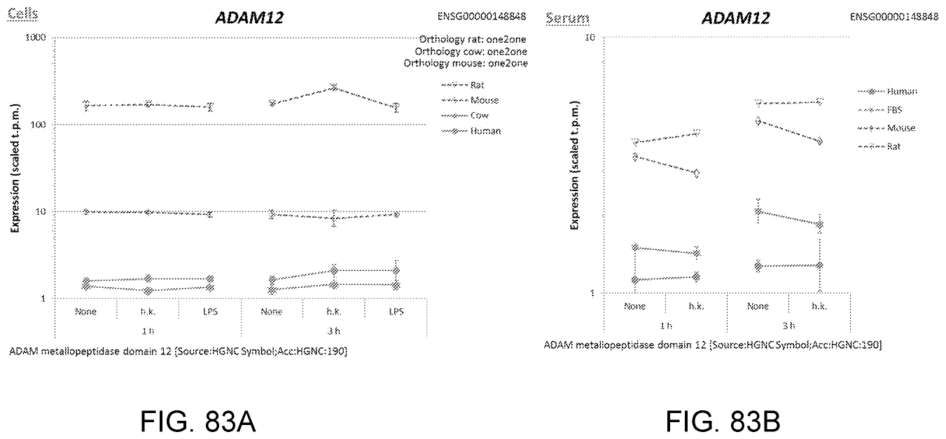

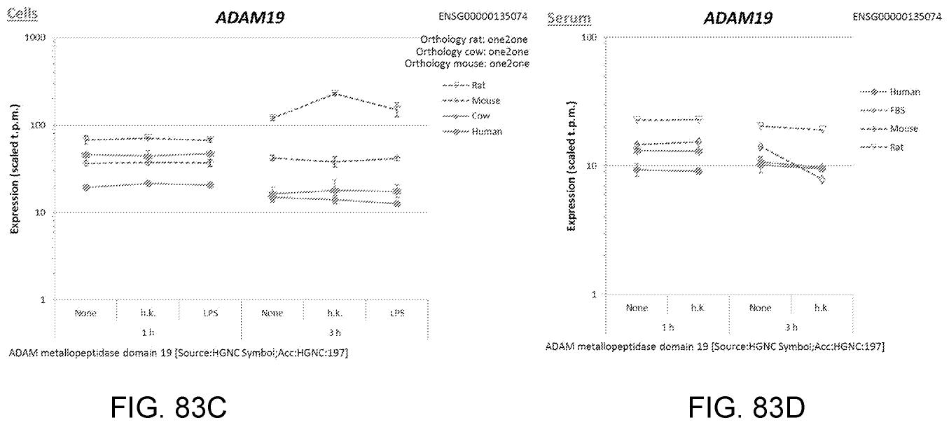

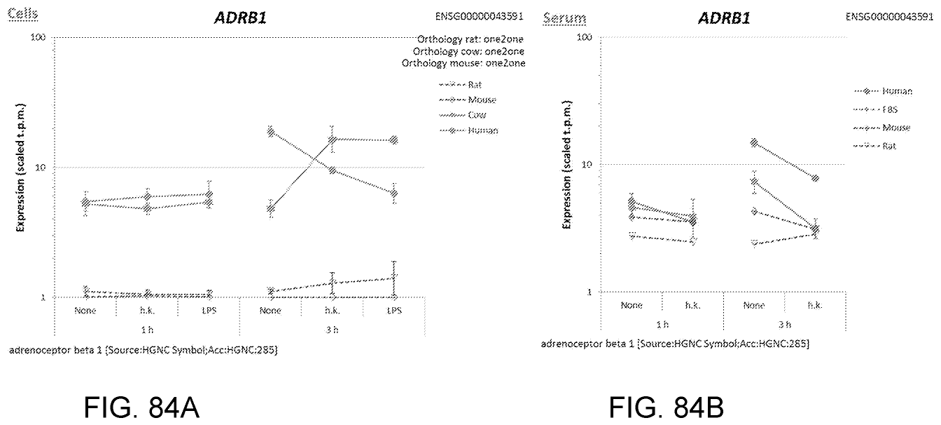

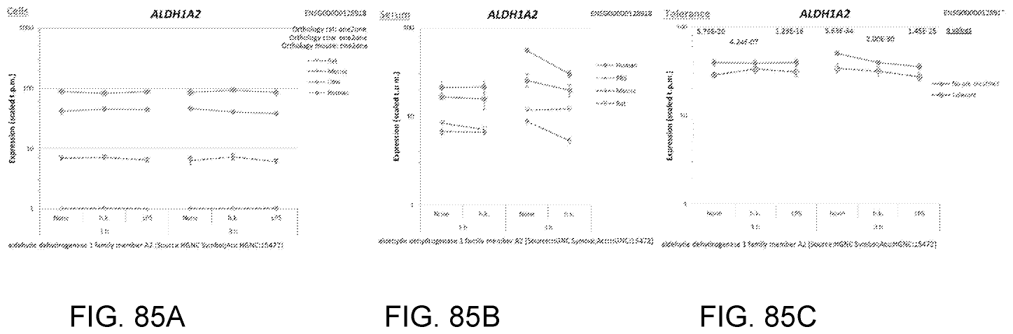

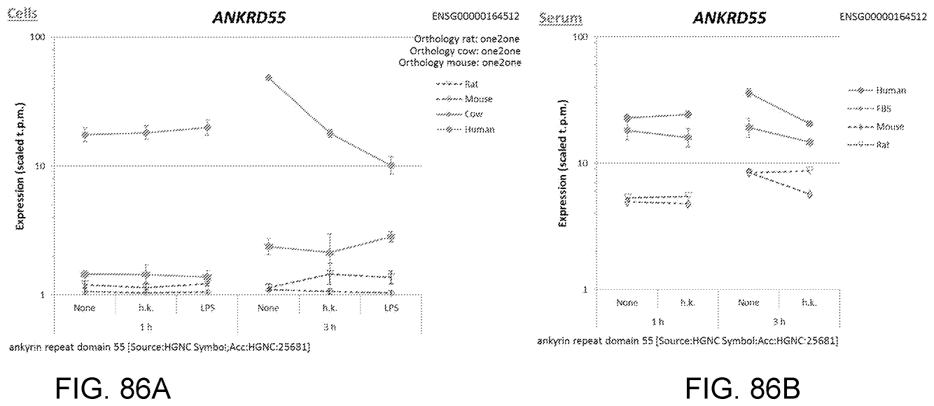

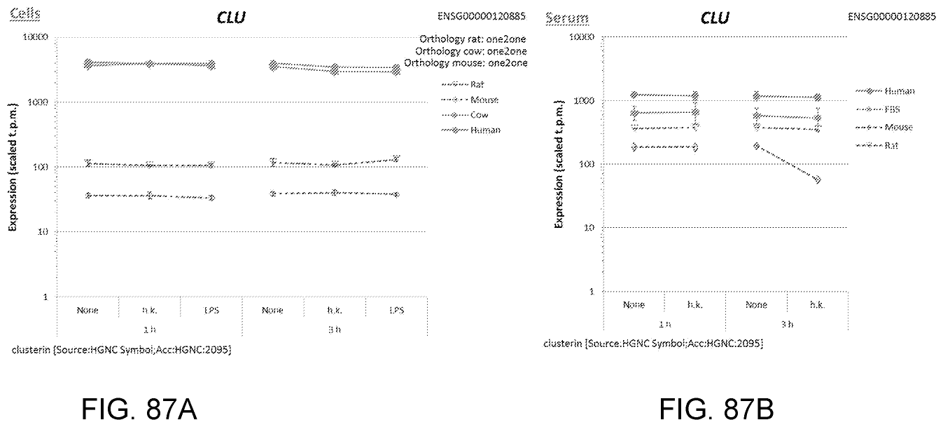

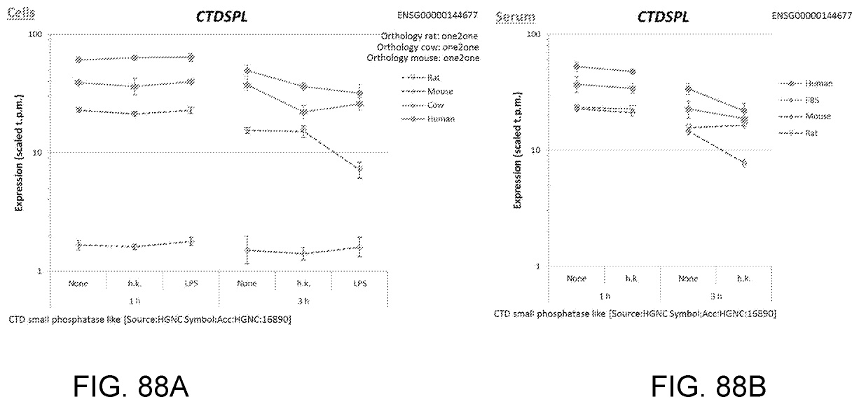

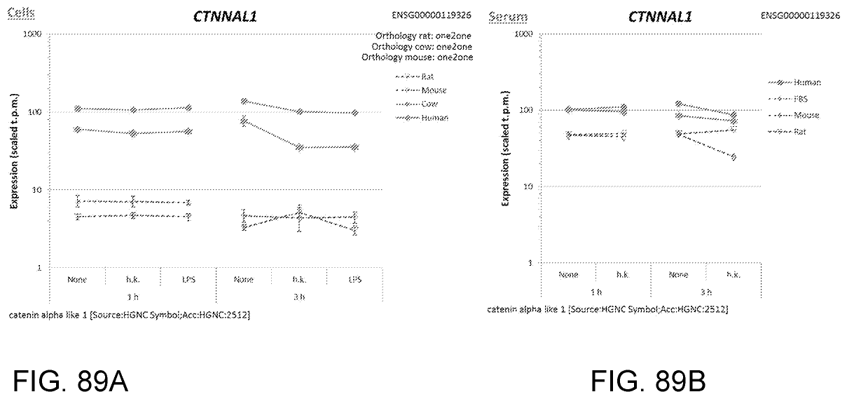

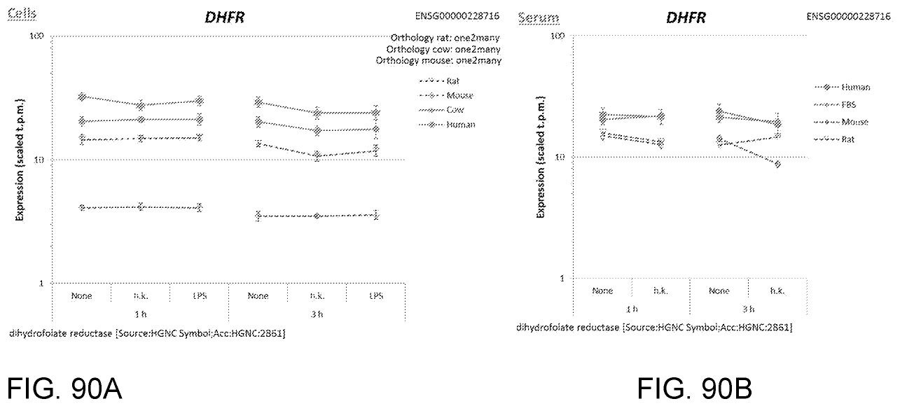

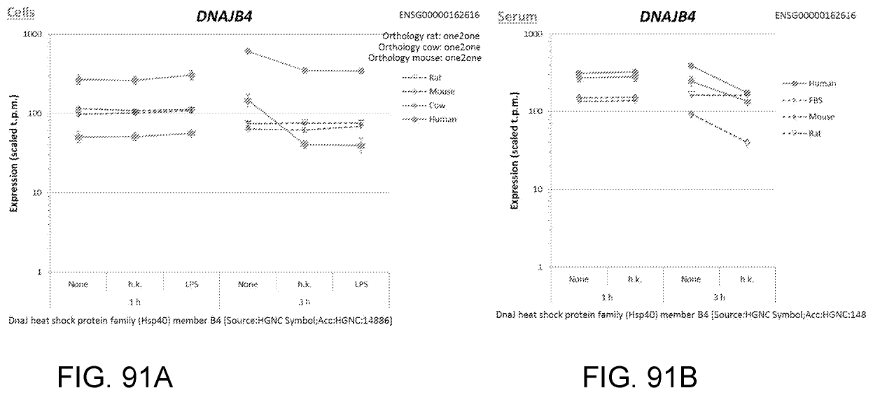

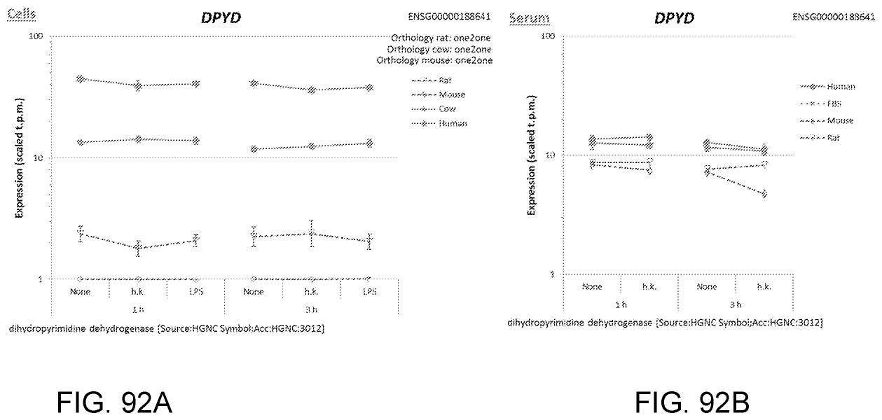

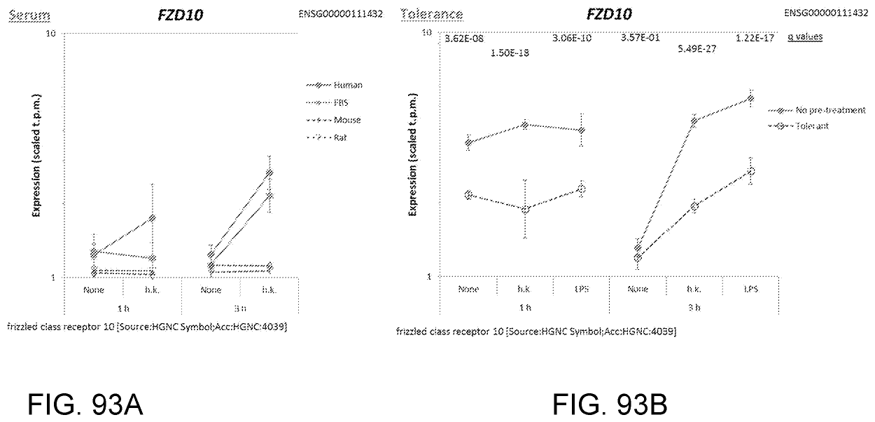

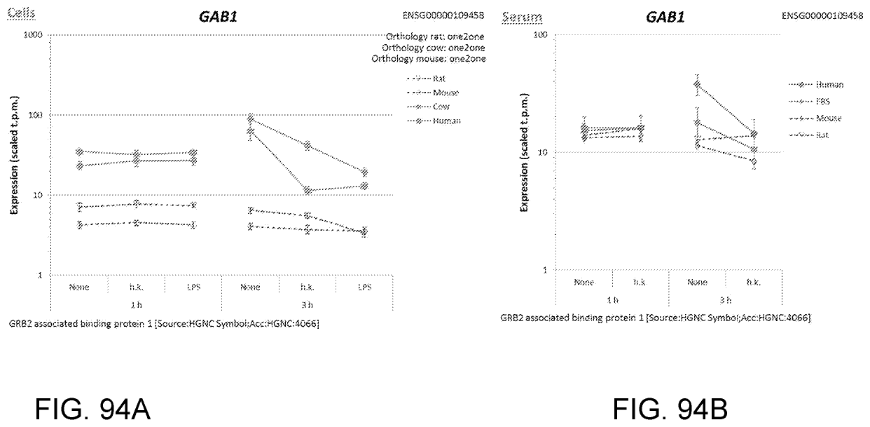

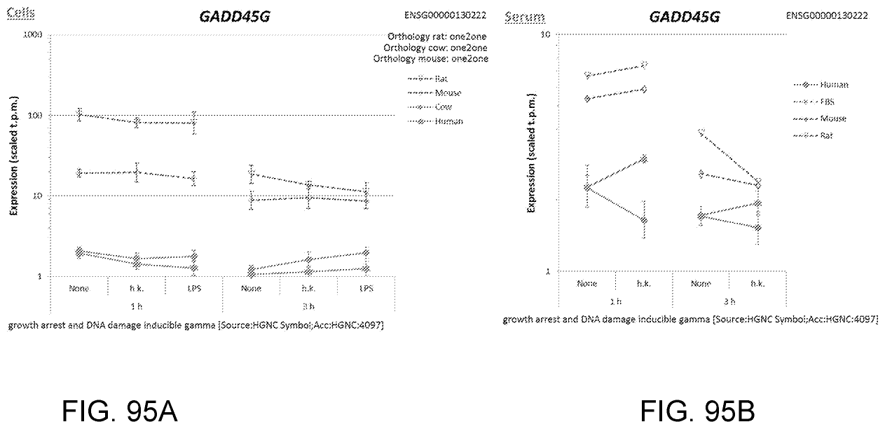

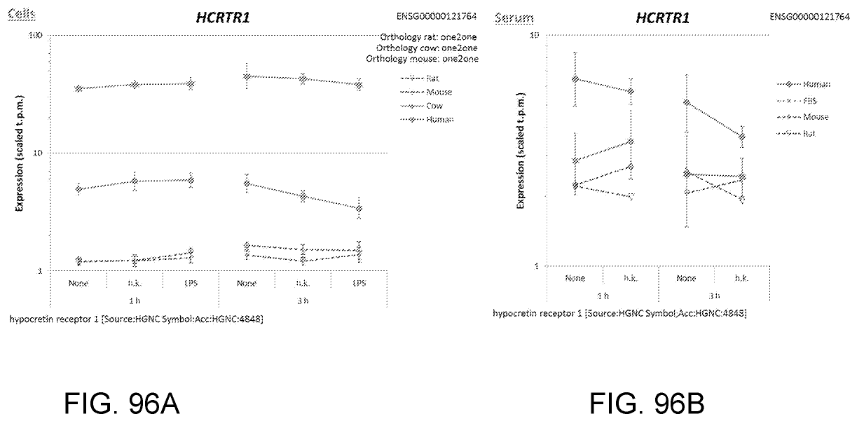

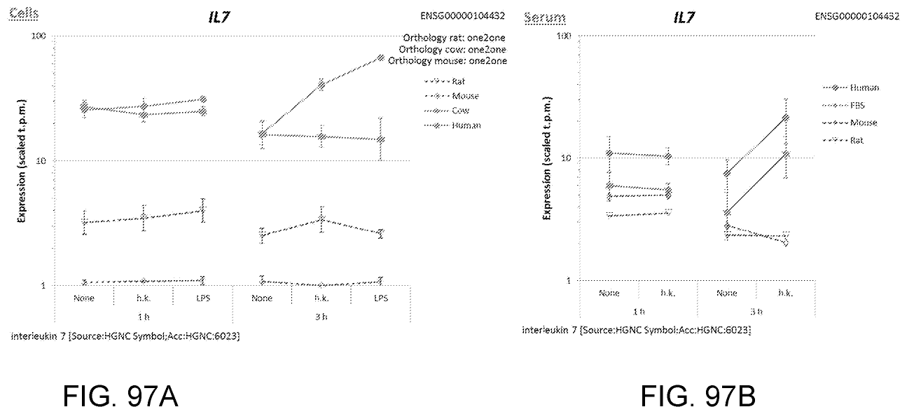

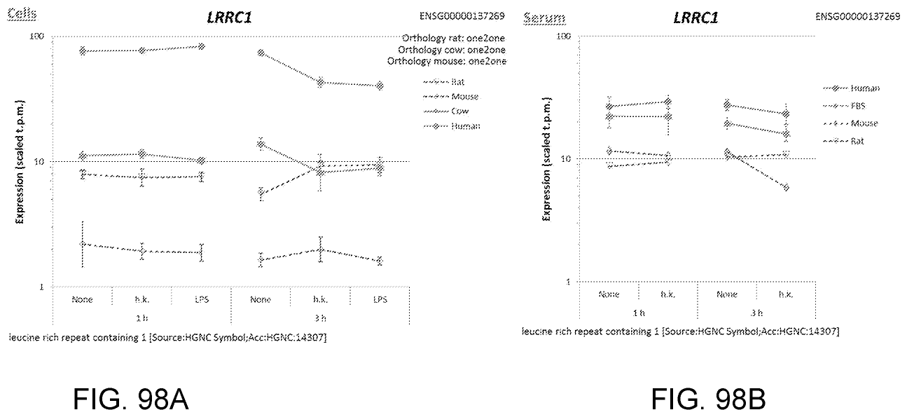

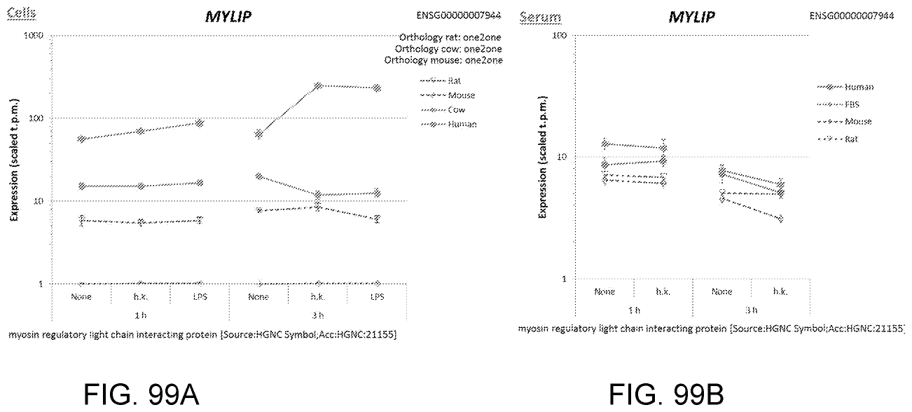

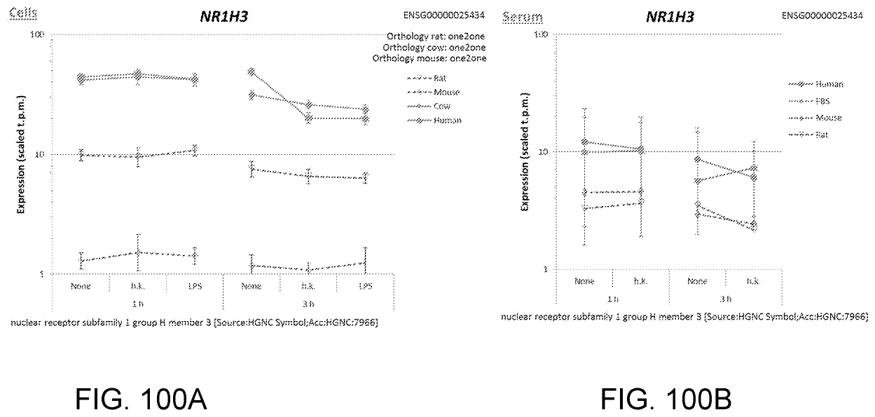

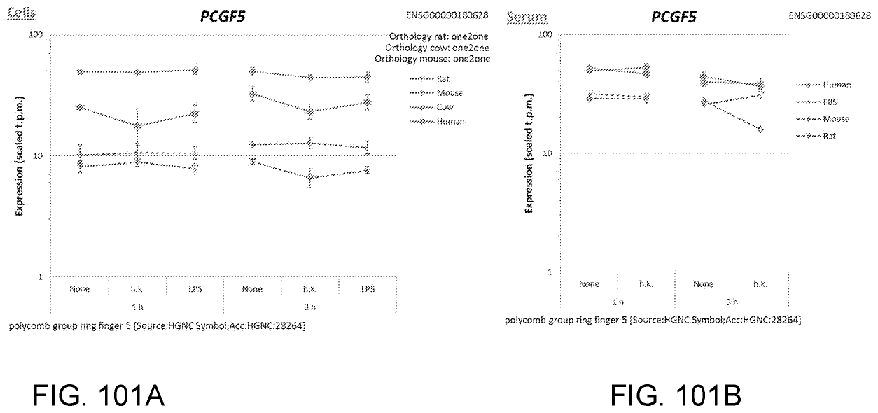

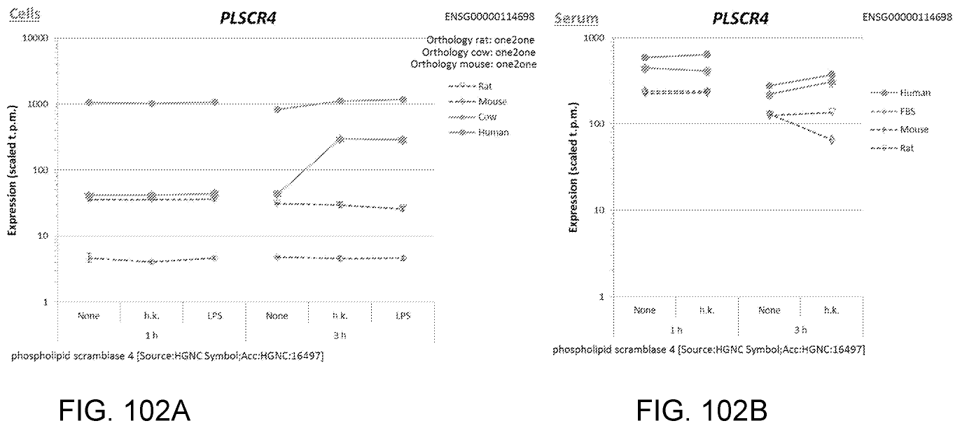

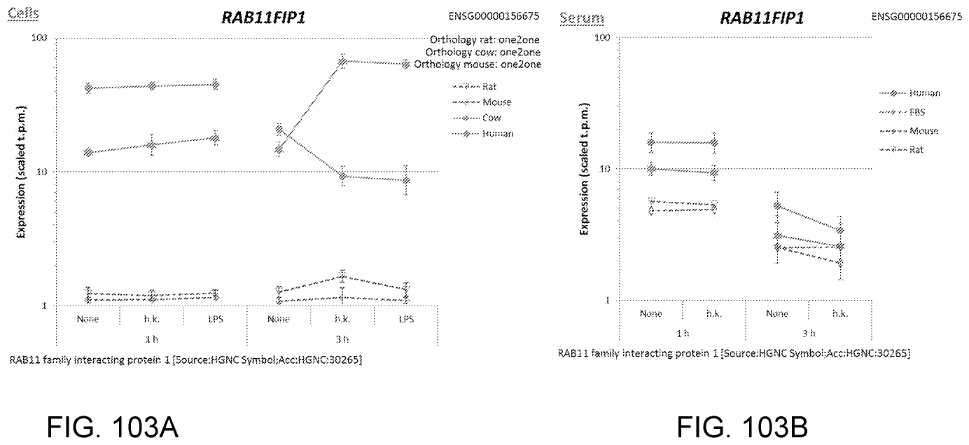

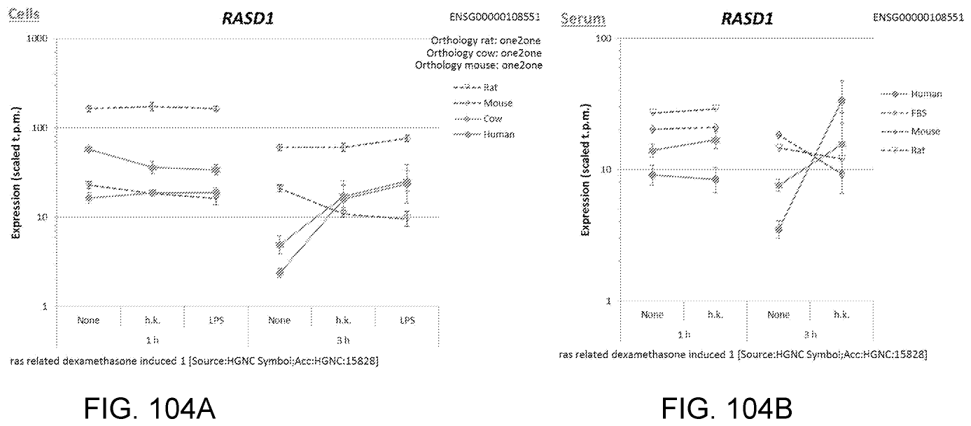

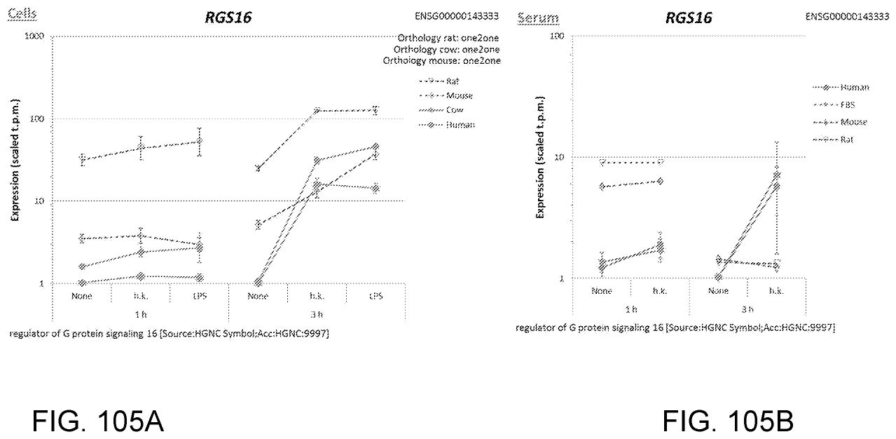

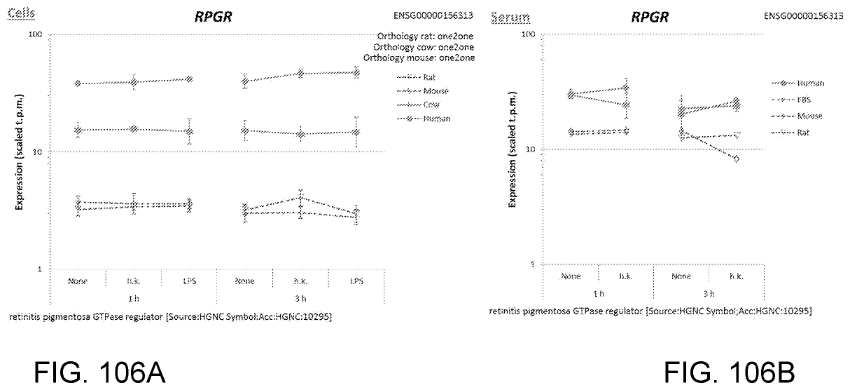

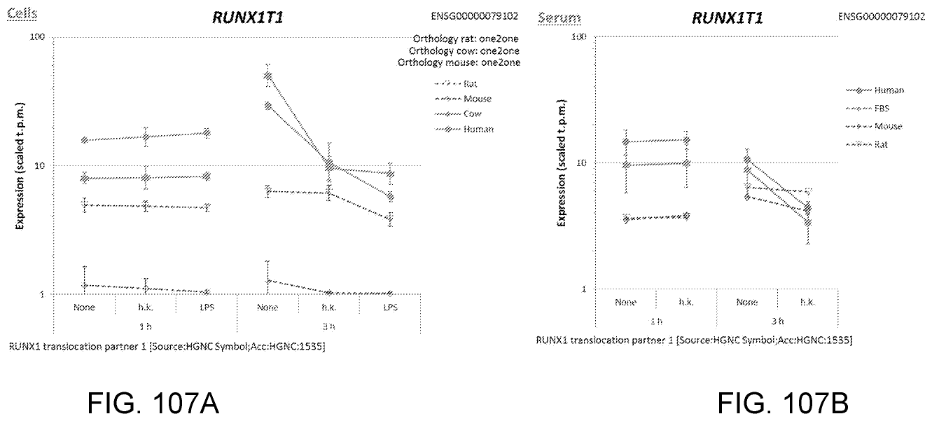

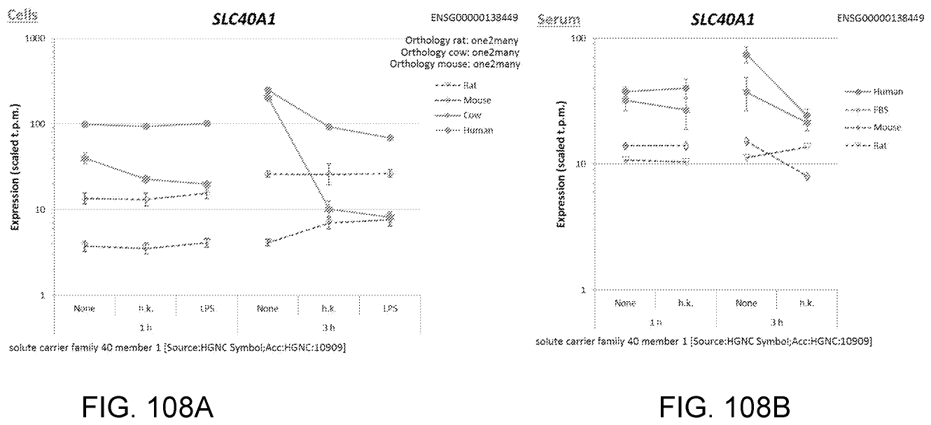

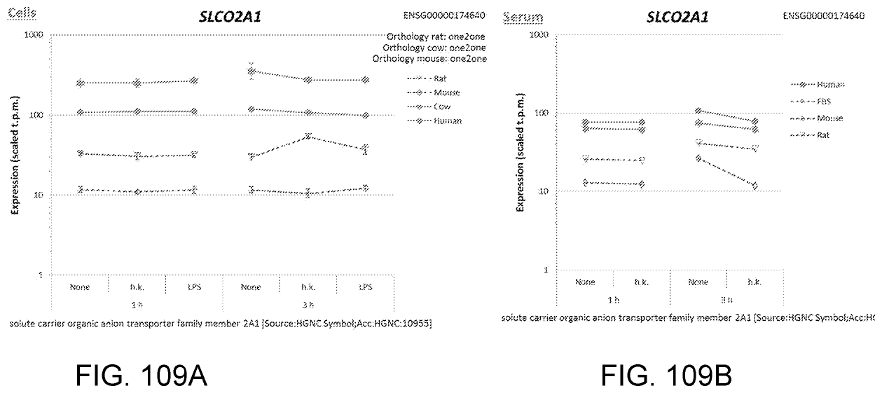

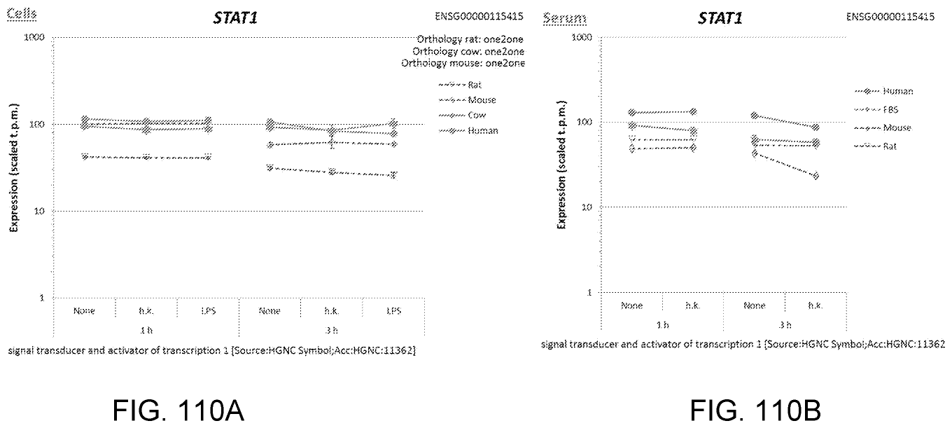

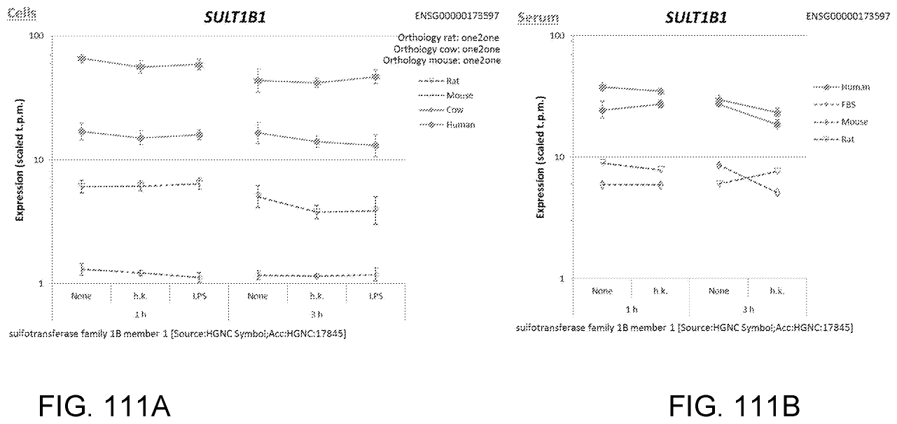

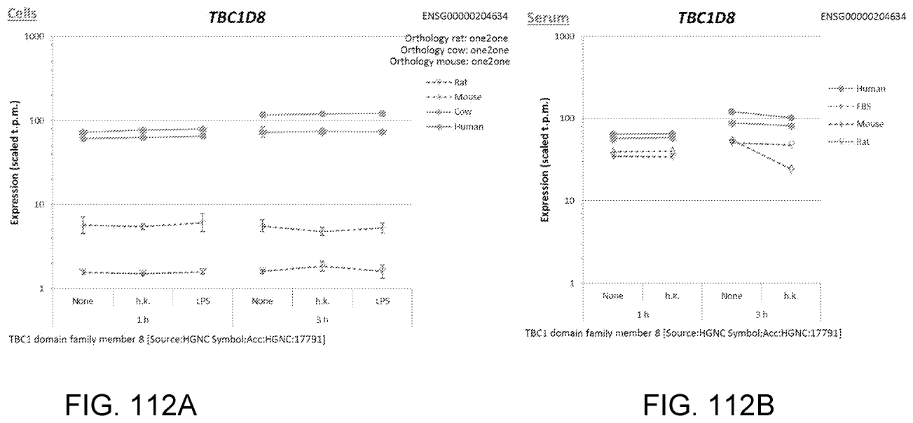

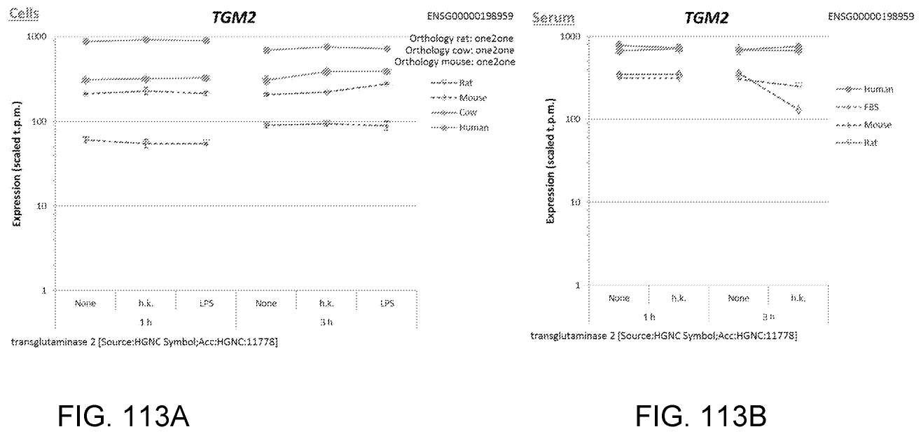

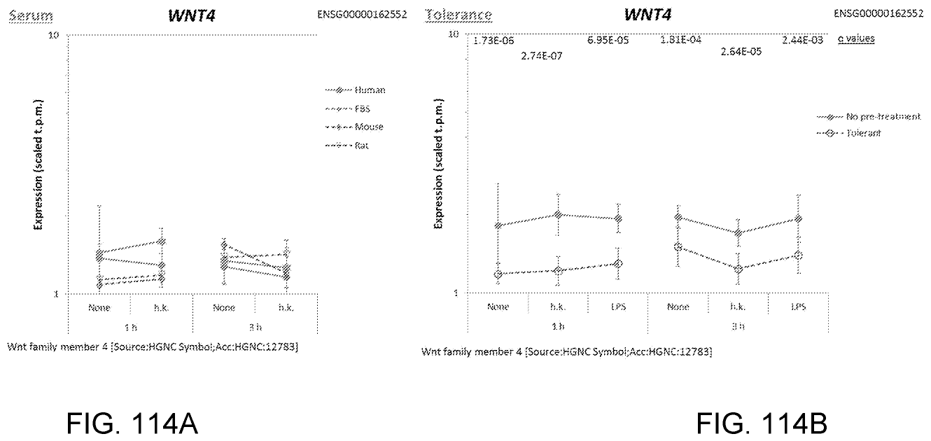

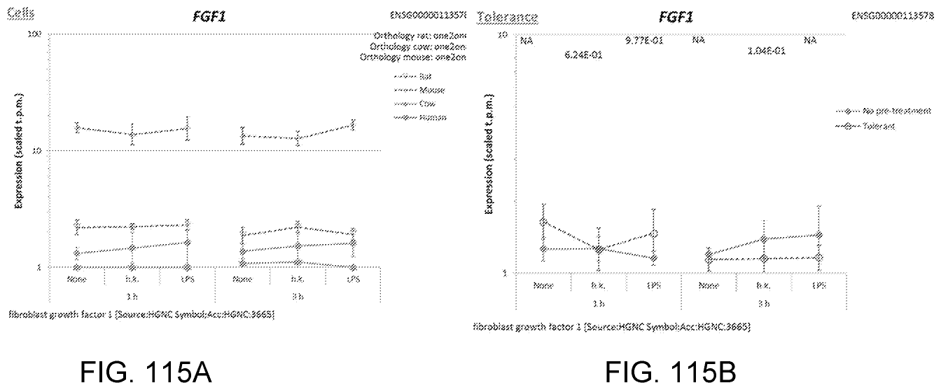

Additional identified targets found using this method include ADAM12 (FIG. 83A-83B), ADAM19 (FIG. 83C-83D), ADRB1(FIG. 84A-84B), ALDH1A2 (FIG. 85A-85C), ANKRD55 (FIG. 86A-86B), CLU (FIG. 87A-87B), CTDSPL (FIG. 88A-88B), CTNNAL1 (FIG. 89A-89B), DHFR (FIG. 90A-90B), DNAJB4 (FIG. 91A-91B), DPYD (FIG. 92A-92B), FZD10 (FIG. 93A-93B), GAB1 (FIG. 94A-94B), GADD45G (FIG. 95A-95B), HCRTR1 (FIG. 96A-96B), IL7 (FIG. 97A-97B), LRRC1 (FIG. 98A-98B), MYLIP (FIG. 99A-99B), NR1H3 (FIG. 100A-100B), PCGF5 (FIG. 101A-101B), PLSCR4 (FIG. 102A-102B), RAB11FIP1 (FIG. 103A-103B), RASD1 (FIG. 104A-104B), RGS16 (FIG. 105A-105B), RPGR (FIG. 106A-106B), RUNX1T1 (FIG. 107A-107B), SLC40A1 (FIG. 108A-108B), SLCO2A1 (FIG. 109A-109B), STAT1 (FIG. 110A-110B), SULT1B1 (FIG. 111A-111B), TBC1D8 (FIG. 112A-112B), TGM2 (FIGS. 113A-113B), and WNT4 (FIGS. 114A-114B), and FGF1 (FIG. 115A-115B).

Genes or gene products associated with a sensitive response are provided in the table below.

TABLE 6

| | | | SEQ |
|---|---|---|---|
| | | | ID |
| | NCBI Name | | NO: |
| 1. | EMC9 | ER Membrane Protein | 1 |
| | [ENSG00000100908] | Complex Subunit 9; Fam158a | |
| 2. | IRAK4 | Interleukin 1 Receptor | 2-3 |
| | [ENSG00000198001] | Associated Kinase 4 | |
| 3. | NFATC2 | Nuclear Factor Of Activated | 4-8 |
| | [ENSG00000101096] | T Cells 2 | |
| 4. | PLA2G10 | Phospholipase A2 Group | 9 |
| | [ENSG00000069764] | X, sPLA₂-X | |
| 5. | SARM1 | Sterile Alpha And TIR Motif | 10-11 |
| | [ENSG00000004139] | Containing 1 | |
| 6. | PIP5K1A | Phosphatidylinositol-4-Phosphate | 12-15 |
| | [ENSG00000143398] | 5-Kinase Type 1 Alpha | |
| 7. | PDIA4 | Protein Disulfide Isomerase | 16 |
| | [ENSG00000155660] | Family A Member 4, Erp72 | |
| 8. | ARPC4 | Actin Related Protein 2/3 | 17-20 |
| | [ENSG00000241553] | Complex Subunit 4 | |
| 9. | BNIP3 | BCL2 Interacting Protein 3 | 21 |
| | [ENSG00000176171] | | |
| 10. | CCDC65 | Coiled-Coil Domain | 22-23 |
| | [ENSG00000139537] | Containing 65 | |
| 11. | CENPH | Centromere Protein H | 24 |
| | [ENSG00000153044] | | |
| 12. | CHPT1 | Choline Phosphotransferase 1 | 25-26 |
| | [ENSG00000111666] | | |
| 13. | COMMD3 | COMM Domain Containing 3 | 27 |
| | [ENSG00000148444] | | |
| 14. | DR1 | Down-Regulator Of | 28 |
| | [ENSG00000117505] | Transcription 1 | |
| 15. | FGD1 | FYVE, RhoGEF And PH | 29 |
| | [ENSG00000102302] | Domain Containing 1 | |
| 16. | FIGNL1 | Fidgetin Like 1 | 30-31 |
| | [ENSG00000132436] | | |
| 17. | GGNBP2 | Gametogenetin Binding | 32-34 |
| | [ENSG00000278311] | Protein 2 | |
| 18. | GNAO1 | G Protein Subunit Alpha O1 | 35-36 |
| | [ENSG00000087258] | | |
| 19. | H2AFZ | H2A Histone Family Member Z | 37 |
| | [ENSG00000164032] | | |
| 20. | HSPB1 | Heat Shock Protein Family B | 38 |
| | [ENSG00000106211] | (Small) Member 1 | |
| 21. | IL17RB | Interleukin 17 Receptor B | 39-40 |
| | [ENSG00000056736] | | |
| 22. | IL17RC | Interleukin 17 Receptor C | 41-48 |
| | [ENSG00000163702] | | |
| 23. | ILF2 | Interleukin Enhancer | 49 |
| | [ENSG00000143621] | Binding Factor 2 | |
| 24. | PDCD6 | Programmed Cell Death 6 | 50-52 |
| | [ENSG00000249915] | | |
| 25. | PI3 | Peptidase Inhibitor 3 | 53 |
| | [ENSG00000124102] | | |
| 26. | POFUT2 | Protein O-Fucosyltransferase 2 | 54-56 |
| | [ENSG00000186866] | | |
| 27. | RABGEF1 | RAB Guanine Nucleotide | |
| | [ENSG00000154710] | Exchange Factor 1 | |
| 28. | RBM4 | RNA Binding Motif Protein 4 | 60-63 |
| | [ENSG00000173933] | | |
| 29. | SKA2 | Spindle And Kinetochore | 64-65 |
| | [ENSG00000182628] | Associated Complex Subunit 2 | |
| 30. | SLC38A2 | Solute Carrier Family 38 | 66-67 |
| | [ENSG00000134294] | Member 2 | |
| 31. | SNRPA1 | Small Nuclear Ribonucleoprotein | 68 |
| | [ENSG00000131876] | Polypeptide A′ | |
| 32. | SPCS2 | Signal Peptidase Complex | 69 |
| | [ENSG00000118363] | Subunit 2 | |
| 33. | TAF1D | TATA-Box Binding Protein | 70 |
| | [ENSG00000166012] | Associated Factor, RNA | |
| | | Polymerase I Subunit D | |
| 34. | TNFSF10 | TNF Superfamily Member | 71-72 |
| | [ENSG00000121858] | 10, TRAIL | |
| 35. | ZNF302 | Zinc Finger Protein 302 | 73-74 |
| | [ENSG00000089335] | | |
| 36. | ZNF333 | Zinc Finger Protein 333 | 75-77 |
| | [ENSG00000160961] | | |
| 37. | ZNF419 | Zinc Finger Protein 419 | 78-83 |
| | [ENSG00000105136] | | |
| 38. | ZNF624 | Zinc Finger Protein 624 | 84-85 |
| | [ENSG00000197566] | | |
| 39. | ZNF677 | Zinc Finger Protein 677 | 86 |
| | [ENSG00000197928] | | |
| 40. | ZNF720 | Zinc Finger Protein 720 | 87-88 |
| | [ENSG00000197302] | | |
| 41. | LPA | Lipoprotein(A), apo(a) | 89 |
| | [ENSG00000198670] | | |
| 42. | B4GAT1 | Beta-1,4-glucuronyltransferase 1 | 90 |
| | [ENSG00000174684] | | |
| 43. | CDH6 | Cadherin 6, type 2, K-cadherin | 91-92 |
| | [ENSG00000113361] | | |
| 44. | CUTA | CutA divalent cation tolerance | 93-95 |
| | [ENSG00000112514] | homolog (E. coli) | |
| 45. | DMBT1 | Deleted in malignant brain | 96-104 |
| | [ENSG00000187908] | tumors 1 | |
| 46. | FCGBP | Fc fragment of IgG binding | 105 |
| | [ENSG00000281123] | protein | |
| 47. | OSMR | Oncostatin M receptor | 106-107 |
| | [ENSG00000145623] | | |
| 48. | SSC5D | Scavenger receptor cysteine rich | 108-109 |
| | [ENSG00000179954] | family, 5 domains | |
| 49. | TNXB | Tenascin XB | 110-113 |
| | [ENSG00000168477] | | |
| 50. | BMP4 | Bone Morphogenetic Protein 4 | 114 |
| | [ENSG00000125378] | | |
| 51. | DPP4 | Dipeptidyl Peptidase 4 | 115 |
| | [ENSG00000197635] | | |
| 52. | MYLK2 | Myosin Light Chain Kinase 2 | 116 |
| | [ENSG00000101306] | | |
| 53. | MYLK4 | Myosin Light Chain Kinase | 117-118 |
| | [ENSG00000145949] | Family Member 4 | |
| 54. | ADRB1 | Adrenoceptor Beta 1 | 119 |
| | [ENSG00000043591] | | |
| 55. | ALDH1A2 | Aldehyde Dehydrogenase 1 | 120-123 |
| | [ENSG00000128918] | Family Member A2 | |
| 56. | ANKRD55 | Ankyrin Repeat Domain 55 | 124-126 |
| | [ENSG00000164512] | | |
| 57. | CLU | Clusterin | 127-132 |
| | [ENSG00000120885] | | |
| 58. | CTDSPL | CTD Small Phosphatase Like | 133-134 |
| | [ENSG00000144677] | | |
| 59. | CTNNAL1 | Catenin Alpha Like 1 | 135-137 |
| | [ENSG00000119326] | | |
| 60. | DHFR | Dihydrofolate Reductase | 138-139 |
| | [ENSG00000228716] | | |
| 61. | DNAJB4 | DnaJ Heat Shock Protein Family | 140 |
| | [ENSG00000162616] | (Hsp40) Member B4 | |
| 62. | DPYD | Dihydropyrimidine | 141-142 |
| | [ENSG00000188641] | Dehydrogenase | |
| 63. | FZD10 | Frizzled Class Receptor 10 | 143 |
| | [ENSG00000111432] | | |
| 64. | GAB1 | GRB2 Associated Binding | 144-145 |
| | [ENSG00000109458] | Protein 1 | |
| 65. | HCRTR1 | Hypocretin Receptor 1 | 146 |
| | [ENSG00000121764] | | |
| 66. | IL7 | Interleukin 7 | 147-149 |
| | [ENSG00000104432] | | |
| 67. | LRRC1 | Leucine Rich Repeat | 150-151 |
| | [ENSG00000137269] | Containing 1 | |
| 68. | MYLIP | Myosin Regulatory Light Chain | 152-153 |
| | [ENSG00000007944] | Interacting Protein | |
| 69. | NR1H3 | Nuclear Receptor Subfamily 1 | 154-156 |
| | [ENSG00000025434] | Group H Member 3 | |
| 70. | PCGF5 | Polycomb Group Ring Finger 5 | 157-158 |
| | [ENSG00000180628] | | |
| 71. | PLSCR4 | Phospholipid Scramblase 4 | 159-160 |
| | [ENSG00000114698] | | |
| 72. | RAB11FIP1 | RAB11 Family Interacting | 161-165 |
| | [ENSG00000156675] | Protein 1 | |
| 73. | RPGR | Retinitis Pigmentosa | 166-171 |
| | [ENSG00000156313] | GTPase Regulator | |
| 74. | RUNX1T1 | RUNX1 Partner Transcriptional | 172-177 |
| | [ENSG00000079102] | Co-Repressor 1 | |
| 75. | SLC40A1 | Solute Carrier Family 40 | 178 |
| | [ENSG00000138449] | Member 1 | |
| 76. | SLCO2A1 | Solute Carrier Organic Anion | 179 |
| | [ENSG00000174640] | Transporter Family Member 2A1 | |
| 77. | STAT1 | Signal Transducer And Activator | 180-181 |
| | [ENSG00000115415] | Of Transcription 1 | |
| 78. | SULT1B1 | Sulfotransferase Family 1B | 182 |
| | [ENSG00000173597] | Member 1 | |
| 79. | TBC1D8 | TBC1 Domain Family | 183-184 |
| | [ENSG00000204634] | Member 8 | |
| 80. | TGM2 | Transglutaminase 2 | 185-187 |
| | [ENSG00000198959] | | |
| 81. | WNT4 | Wnt Family Member 4 | 188-189 |
| | [ENSG00000162552] | | |

Genes or gene products associated with a resistant response are provided in the table below.

TABLE 7

| | | | SEQ |
|---|---|---|---|
| | NCBI Name | | ID NO: |
| 1. | ARHGEF10L | Rho Guanine Nucleotide | 190-194 |
| | [ENSG00000074964] | Exchange Factor 10 | |
| | | Like; GrinchGEF | |
| 2. | SLC8A1 | Solute Carrier Family 8 | 195-199 |
| | [ENSG00000183023] | Member A1; NCX1 | |
| 3. | TREML1 | Triggering Receptor | 200-202 |
| | [ENSG00000161911] | Expressed On Myeloid | |
| | | Cells Like 1 | |
| 4. | PEAR1 | Platelet Endothelial | 203 |
| | [ENSG00000187800] | Aggregation Receptor 1 | |
| 5. | SFN | Stratifin | 204-205 |
| | [ENSG00000175793] | | |
| 6. | CD14 | CD14 Molecule | 206 |
| | [ENSG00000170458] | | |
| 7. | AOAH | Acyloxyacyl Hydrolase | 207-208 |
| | [ENSG00000136250] | | |
| 8. | ASPRV1 | Aspartic Peptidase | 209 |
| | [ENSMUSG00000033508] | Retroviral Like 1 | |
| 9. | EHD1 | EH Domain Containing 1 | 210 |
| | [ENSG00000110047] | | |
| 10. | LCN2 | Lipocalin 2 | 211 |
| | [ENSMUSG00000026822] | | |
| 11. | ST3GAL6 | ST3 Beta-Galactoside Alpha- | 212-213 |
| | [ENSG00000064225] | 2,3-Sialyltransferase 6 | |
| 12. | MFSD1 | Major Facilitator Superfamily | 214-219 |
| | [ENSG00000118855] | Domain Containing 1 | |
| 13. | PECAM1 | Platelet and Endothelial Cell | 220-225 |
| | [ENSG00000261371 | Adhesion Molecule 1 | |
| 14. | ESAM | Endothelial Cell | 226-227 |
| | [ENSG00000149564] | Adhesion Molecule | |
| 15. | AFM | Afamin | 228 |
| | [ENSG00000079557] | | |
| 16. | APCS | Amyloid P component, | 229 |
| | [ENSG00000132703] | serum | |
| 17. | F12 | Coagulation factor XII | 230 |
| | [ENSG00000131187] | (Hageman factor) | |
| 18. | GCA | Grancalcin | 231 |
| | [ENSG00000115271] | | |
| 19. | GLOD4 | Glyoxalase domain | 232-234 |
| | [ENSG00000167699] | containing 4 | |
| 20. | GPS | Glycoprotein V (platelet) | 235 |
| | [ENSG00000178732] | | |
| 21. | HGFAC | HGF activator | 236 |
| | [ENSG00000109758] | | |
| 22. | IGF1 | Insulin like growth factor 1 | 237-240 |
| | [ENSG00000017427] | | |
| 23. | MMRN2 | Multimerin 2 | 241 |
| | [ENSG00000173269] | | |
| 24. | PF4V1 | Platelet Factor 4 Variant 1 | 242 |
| | [ENSG00000109272] | | |
| 25. | PFKL | Phosphofructokinase, liver | 243-244 |
| | [ENSG00000141959] | | |
| 26. | POSTN | Periostin, osteoblast | 245-251 |
| | [ENSG00000133110] | specific factor | |
| 27. | SAA4 | Serum amyloid A4 | 252 |
| | [ENSG00000148965] | | |
| 28. | TIMP3 | TIMP metallopeptidase | 253 |
| | [ENSG00000100234] | inhibitor 3 | |
| 29. | YWHAG | Tyrosine 3-monooxygenase/ | 254 |
| | [ENSG00000170027] | tryptophan 5-monooxygenase | |
| | | activation protein, gamma | |
| 30. | ADAM12 | ADAM Metallopeptidase | 255-258 |
| | [ENSG00000148848] | Domain 12 | |
| 31. | ADAM19 | ADAM Metallopeptidase | 259-260 |
| | [ENSG00000135074] | Domain 19 | |
| 32. | FGF1 | Fibroblast growth factor 1 | 261-262 |
| | [ENSG00000113578] | | |
| 33. | GADD45G | Growth Arrest and DNA | 263 |
| | [ENSG00000130222] | Damage Inducible Gamma | |
| 34. | RASD1 | Ras Related Dexamethasone | 264-265 |
| | [ENSG00000108551] | Induced 1 | |
| 35. | RGS16 | Regulator of G Protein | 266 |
| | [ENSG00000143333] | Signaling 16 | |

All publications (including patents and patent applications including U.S. provisional application Ser. Nos. 62/794,386 and 62/930,813) mentioned in this specification are herein incorporated by reference to the same extent as if each individual publication or patent was specifically and individually indicated to be incorporated by reference.

### Other Embodiments

From the foregoing description, it will be apparent that variations and modifications may be made to the invention described herein to adopt it to various usages and conditions. Such embodiments are also within the scope of the following claims.

What is claimed is:

1. A method for treating a disease of immune dysregulation in a mammal, the method comprising administering to the mammal a therapeutically effective amount of a modulating agent or subjecting the mammal to a modulating process, wherein the modulating agent or process alters the expression or activity of a gene target associated with a sensitive response or a resistant response to an inflammatory stimulus.

2. A method for altering an immune response in a mammal, the method comprising administering to the mammal a therapeutically effective amount of a modulating agent or subjecting the mammal to a modulating process, wherein the modulating agent or process alters the expression or activity of a gene target associated with a sensitive response or a resistant response to an inflammatory stimulus.

3. The method of claim 1 or 2, wherein the gene target is a gene or gene product associated with a sensitive response.

4. The method of claim 3, wherein the gene is EMC9 or the gene product is an RNA or polypeptide encoded by EMC9.

5. The method of claim 1 or 2, wherein the gene target is a gene or gene product associated with a resistant response.

6. The method of claim 5, wherein the gene is ARHGEF10L or the gene product is an RNA or polypeptide encoded by ARHGEF10L.

7. The method of claim 1, wherein the modulating agent or process increases the expression or activity of the gene target.

8. The method of claim 7, wherein the modulating agent is an activator of the gene target, an agonist antibody of the gene target, a cell expressing the gene target, a mimetic of the gene target, a derivative or recombinant form of the gene target, or a soluble form of the gene target.

9. The method of claim 7, wherein the modulating process is overexpression of the gene target or overexpression or depletion of a signal, signaling regulator, or receptor associated with the gene target.

10. The method of claim 1, wherein the modulating agent or process decreases the expression or activity of the gene target.

11. The method of claim 10, wherein the modulating agent is an inhibitor of the gene target, an inhibiting or neutralizing antibody of the gene target, a moiety blocking a receptor associated with the gene target, or a RNAi molecule targeting the gene target.

12. The method of claim 10, wherein the modulating process is depletion of cells expressing the gene target, overexpression or depletion of a signal, signaling regulator, or receptor associated with the gene target, depletion of a ligand of the gene target, or genetic ablation of the gene target.

13. The method of claim 1, wherein the modulating agent is a protein, a peptide, a polynucleotide, a small molecule, or a chemical.

14. The method of claim 1, wherein the modulating agent is delivered orally, by injection, by a lipid-based carrier, or by a nanoparticle-type carrier.

15. The method of claim 1, wherein the modulating agent is delivered by an expression vector or plasmid containing a gene insert that codes for the immunomodulant agent.

16. The method of claim 1, wherein the modulating agent is associated with a gene editing technology.

17. The method of claim 1, wherein the inflammatory stimulus is a bacterial lipopolysaccharide (LPS).

18. The method of claims 1 and 3, wherein the disease of immune dysregulation is an inflammatory disease.

19. The method of claim 18, wherein the inflammatory disease is a dermatological disorder.

20. The method of claim 19, wherein the dermatological disorder is atopic dermatitis, alopecia areata, bulloid pemphigus, chronic eczema, dermatomyositis, erythema nodosum, epidermolysis bullosa, hydradenitis suppurativa, lichen planus, pemphigus vulgaris, psoriasis, pyoderma gangrenosum, scleroderma, or vitiligo.

21. The method of claim 18, wherein the inflammatory disease is sepsis, achalasia, acute or ischemic colitis, acute respiratory distress syndrome, allergy, allograft rejection, alveolitis, Alzheimer's disease, a neurological disease associated with amyloidosis, amebiasis, anaphylactic shock, angiitis, ankylosing spondylitis, appendicitis, arteritis, arthralgia, arthritides, asthma, atherosclerosis, Behcet's syndrome, Berger's disease, bronchiolitis, bronchitis, burns, cachexia, candidiasis, cerebral embolism, cerebral infarction, cholangitis, cholecystitis, chronic fatigue syndrome, celiac disease, Crohn's disease, congestive heart failure, Crohn's disease, cystic fibrosis, Dengue fever, dermatitis, dermatomyositis, disseminated bacteremia, diverticulitis, duodenal ulcers, emphysema, encephalitis, endocarditis, endotoxic shock, enteritis, eosinophilic granuloma, epididymitis, epiglottitis, fasciitis, filariasis, gastric ulcers, Goodpasture's syndrome, gout, graft-versus-host disease, granulomatosis, Guillan-Barre syndrome, hay fever, hepatitis, hepatitis B virus infection, hepatitis C virus infection, herpes infection, HIV infection, Hodgkin's disease, hydatid cysts, hyperpyrexia, immune complex disease, influenza, juvenile idiopathic arthritis, malaria, meningitis, multiple sclerosis, a multiple sclerosis-associated demyelination disease, myasthenia gravis, myocardial ischemia, myocarditis, neuralgia, neuritis, organ ischemia, organ necrosis, osteomyelitis, Paget's disease, pancreatitis, ulcerative pancreatitis, pseudomembranous colitis, pancreatitis, paralysis, peptic ulcers, periarteritis nodosa, pericarditis, periodontal disease, peritonitis, pharyngitis, pleurisy, pneumonitis, pneumonoultramicroscopicsilicovolcanokoniosis, polymyalgia rheumatica, prostatitis, psoriatic arthritis, pseudomembranous Reiter's syndrome, reperfusion injury, respiratory syncytial virus infection, rheumatic fever, rheumatoid arthritis, rhinitis, sarcoidosis, septic abortion, septicemia, sinusitis, forms of cancer having an inflammatory component, spinal cord injury, sunburn, synovitis, systemic lupus erythematosus, systemic lupus erythrocytosis, thrombophlebitis, thyroiditis, Type I diabetes, ulcerative colitis, urethritis, urticaria, uveitis, vaginitis, vasculitis, warts, wheals, or Whipple's disease.

22. The method of claim 18, wherein the inflammatory disease is sepsis.

23. The method of claims 1 and 3, wherein the disease of immune dysregulation is secondary induced inflammation.

24. The method of claim 23, wherein the secondary induced inflammation is associated with a bacterial infection, a viral infection, a fungal infection, or a parasitic infection.

25. The method of claim 18, wherein the inflammatory disease is allograft rejection and wherein the composition further comprises an immunosuppressant used to inhibit allograft rejection.

26. The method of claim 18, wherein the inflammatory disease is a xenograft rejection and wherein the composition further comprises an immunosuppressant used to treat xenograft rejection.

27. The method of claim 2, where the mammal has a cancer and the alteration of the expression or activity of the gene target increases an innate immune response of the mammal.

28. The method of claim 1, wherein the mammal is a human.

29. A method for identifying one or more effective therapeutic intervention targets for a disease of immune dysregulation, the method comprising:
(iii) measuring a whole transcriptome gene expression profile in leukocytes from a whole blood sample of a mammal, wherein the mammal or the whole blood sample has been treated with a pro-inflammatory stimulus or has not been treated with an inflammatory stimulus and wherein the mammal has an in vivo innate immune response that is resistant or sensitive, and
(iv) identifying the whole-transcriptome gene expression profile as associated with innate immune resistance or sensitivity based on the in vivo innate immune response of the mammal,
wherein the gene expression profiles are associated with innate immune resistance or sensitivity and are potential therapeutic targets for diseases of innate immune dysregulation.

30. A method for identifying a therapeutic intervention target for a disease of immune dysregulation, the method comprising:
(a) determining a gene expression profile in a blood sample of a first mammal, wherein the first mammal has an in vivo innate immune response that is resistant,
(b) determining a gene expression profile in a blood sample of a second mammal, wherein the second mammal has an in vivo innate immune response that is sensitive,
(c) identifying a gene or gene target having differential expression between the first mammal and the second mammal, and
(d) identifying said gene or gene target as associated with a resistant response or a sensitive response based on said differential expression,
wherein a gene or gene target associated with a resistant response or a sensitive response is identified as a therapeutic intervention target for a disease of immune dysregulation.

31. The method of claim 30, wherein the first mammal and the second mammal, or the whole blood samples thereof, have not been treated with an inflammatory stimulus.

32. The method of claim 30, wherein the first mammal and the second mammal, or the whole blood samples thereof, have been treated with an inflammatory stimulus and the differential expression is differential expression following exposure to the inflammatory stimulus.

33. The method of claim 32, wherein the inflammatory stimulus is a toxin such as LPS or a viral mimic.

34. The method of claim 32, wherein the viral mimic is polyinosinic:polycytidylic acid (Poly(I:C)).

35. The method of claim 30, wherein the first mammal is one or more of baboon, rhesus monkey (rhesus macaque), rat, or mouse.

36. The method of claim 30, wherein the second mammal is one or more of human, chimp, rabbit, sheep, cow, or pig.

37. A method for assessing an immune response of a mammal, the method comprising determining the expression of a gene target associated with a sensitive response or a resistant response to an inflammatory stimulus in the mammal, wherein the expression of the gene target identifies the immune response of the mammal as a sensitive response or a resistant response.

38. The method of claim 37, wherein the gene target is a gene or gene product associated with a resistant response.

39. The method of claim 38, wherein the gene is one or more of ARHGEF10L, SLC8A1, TREML1, PEAR1, SFN, CD14, AOAH, ASPRV1, EHD1, LCN2, ST3GAL6, MFSD1, PECAM1, ESAM, AFM, APCS, F12, GCA, GLOD4, GP5, HGFAC, IGF1, MMRN2, PF4V1, PFKL, POSTN, SAA4, TIMP3, YWHAG, ADAM12, ADAM19, GADD45G, FGF1, RASD1 or RGS16, or the gene product is an RNA or polypeptide encoded by one or more of the aforementioned genes.

40. The method of claim 39, wherein the expression level of one or more of ARHGEF10L, SLC8A1, TREML1, PEAR1, SFN, CD14, AOAH, ASPRV1, EHD1, LCN2, ST3GAL6, MFSD1, PECAM1, ESAM, AFM, APCS, F12, GCA, GLOD4, GP5, HGFAC, IGF1, MMRN2, PF4V1, PFKL, POSTN, SAA4, TIMP3, YWHAG, ADAM12, ADAM19, GADD45G, FGF1, RASD1 or RGS16 is determined.

41. The method of claim 39, wherein the expression level of one or more of ARHGEF10L, SLC8A1, TREML1, PEAR1, SFN, CD14, AOAH, ASPRV1, EHD1, LCN2, ST3GAL6, MFSD1, PECAM1, ESAM, AFM, APCS, or F12 is determined.

42. The method of claim 37, wherein the gene target is a gene or gene product associated with a sensitive response.

43. The method of claim 41, wherein the gene target is gene is one or more of EMC9, IRAK4, NFATC2, PLA2G10, SARM1, PIP5K1A, PDIA4, ARPC4, BNIP3, CCDC65, CENPH, CHPT1, COMMD3, DR1, FGD1, FIGNL1, GGNBP2, GNAO1, H2AFZ, HSPB1, IL17RB, IL17RC, ILF2, PDCD6, PI3, POFUT2, RABGEF1, RBM4, SKA2, SLC38A2, SNRPA1, SPCS2, TAF1D, TNFSF10, ZNF302, ZNF333, ZNF419, ZNF624, ZNF677, ZNF720, LPA, B4GAT1, CDH6, CUTA, DMBT1, FCGBP, OSMR, SSC5D, TNXB, BMP4, DPP4, MYLK2, MYLK4, ADRB1, ALDH1A2, ANKRD55, CLU, CTDSPL, CTNNAL1, DHFR, DNAJB4, DPYD, FZD10, GAB1, HCRTR1, IL7, LRRC1, MYLIP, NR1H3, PCGF5, PLSCR4, RAB11FIP1, RPGR, RUNX1T1, SLC40A1, SLCO2A1, STAT1, SULT1B1, TBC1D8, TGM2 or WNT4, or the gene product is an RNA or polypeptide encoded by one or more of the aforementioned genes.

44. The method of claim 43, wherein the expression level of one or more of EMC9, IRAK4, NFATC2, PLA2G10, SARM1, PIP5K1A, PDIA4, ARPC4, BNIP3, CCDC65, CENPH, CHPT1, COMMD3, DR1, FGD1, FIGNL1, GGNBP2, GNAO1, H2AFZ, HSPB1, IL17RB, IL17RC, ILF2, PDCD6, PI3, POFUT2, RABGEF1, RBM4, SKA2, SLC38A2, SNRPA1, SPCS2, TAF1D, TNFSF10, ZNF302, ZNF333, ZNF419, ZNF624, ZNF677, ZNF720, LPA, B4GAT1, CDH6, CUTA, DMBT1, FCGBP, OSMR, SSC5D, TNXB, BMP4, DPP4, MYLK2, MYLK4, ADRB1, ALDH1A2, ANKRD55, CLU, CTDSPL, CTNNAL1, DHFR, DNAJB4, DPYD, FZD10, GAB1, HCRTR1, IL7, LRRC1, MYLIP, NR1H3, PCGF5, PLSCR4, RAB11FIP1, RPGR, RUNX1T1, SLC40A1, SLCO2A1, STAT1, SULT1B1, TBC1D8, TGM2 or WNT4 is determined.

45. The method of claim 43, wherein the expression level of one or more of EMC9, IRAK4, NFATC2, PLA2G10, SARM1, PIP5K1A, PDIA4, ARPC4, BNIP3, CCDC65, CENPH, CHPT1, COMMD3, DR1, FGD1, FIGNL1, GGNBP2, GNAO1, H2AFZ, HSPB1, IL17RB, IL17RC, ILF2, PDCD6, PI3, POFUT2, RABGEF1, RBM4, SKA2, SLC38A2, SNRPA1, SPCS2, TAF1D, TNFSF10, ZNF302, ZNF333, ZNF419, ZNF624, ZNF677, ZNF720, LPA, B4GAT1, CDH6, CUTA, DMBT1, or FCGBP is determined.

46. The method of claim 43, wherein the expression level of one or more of EMC9, IRAK4, NFATC2, PLA2G10, SARM1, PIP5K1A, PDIA4, ARPC4, BNIP3, CCDC65, CENPH, CHPT1, COMMD3, DR1, FGD1, FIGNL1, GGNBP2, GNAO1, H2AFZ, or HSPB1 is determined.

47. The method of claim 37, wherein the expression level is an mRNA expression level.

48. The method of claim 47, wherein the mRNA expression level is determined by PCR, RT-PCR, RNA-seq, gene expression profiling, serial analysis of gene expression, or microarray analysis.

49. The method of claim 48, wherein the mRNA expression level is determined by RNA-seq.

50. The method of claim 37, wherein the expression level is a protein expression level.

51. The method of claim 50, wherein the protein expression is determined by western blot, immunohistochemistry, or mass spectrometry.

52. A method for treating a disease of immune dysregulation in a mammal, the method comprising administering to the mammal a therapeutically effective amount of a modulating agent or subjecting the mammal to a modulating process, wherein the modulating agent or process alters the expression or activity of EMC9 to an inflammatory stimulus.

53. A method for altering an immune response in a mammal, the method comprising administering to the mammal a therapeutically effective amount of a modulating agent or subjecting the mammal to a modulating process, wherein the modulating agent or process alters the expression or activity of EMC9 to an inflammatory stimulus.

54. The method of claim 52 or 53, wherein the modulating agent or process increases the expression or activity of EMC9.

55. The method of claim 54, wherein the modulating agent is an activator of EMC9, an agonist antibody of EMC9, a cell expressing EMC9, a mimetic of EMC9, a derivative or recombinant form of EMC9, or a soluble form of EMC9.

56. The method of claim 55, wherein the modulating process is overexpression of EMC9 or overexpression or depletion of a signal, signaling regulator, or receptor associated with EMC9.

57. The method of claim 52, wherein the modulating agent or process decreases the expression or activity of EMC9.

58. The method of claim 57, wherein the modulating agent is an inhibitor of EMC9, an inhibiting or neutralizing antibody of EMC9, a moiety blocking a receptor associated with the gene target, or a RNAi molecule targeting EMC9.

59. The method of claim 58, wherein the modulating process is depletion of cells expressing EMC9, overexpression or depletion of a signal, signaling regulator, or receptor associated with EMC9, depletion of a ligand of EMC9, or genetic ablation of EMC9.

60. The method of claim 52 or claim 53, wherein the modulating agent is a protein, a peptide, a polynucleotide, a small molecule, or a chemical.

61. The method of claim 52 or claim 53, wherein the modulating agent is delivered orally, by injection, by a lipid-based carrier, or by a nanoparticle-type carrier.

62. The method of claim 52 or claim 53, wherein the modulating agent is delivered by an expression vector or plasmid containing a gene insert that codes for the modulating agent.

63. The method of claim 52 or claim 53, wherein the modulating agent is associated with a gene editing technology.

64. The method of claim 52 or claim 53, wherein the inflammatory stimulus is a bacterial lipopolysaccharide (LPS).

65. The method of claim 52 or claim 54, wherein the disease of immune dysregulation is an inflammatory disease.

66. The method of claim 65, wherein the inflammatory disease is a dermatological disorder.

67. The method of claim 66, wherein the dermatological disorder is atopic dermatitis, alopecia areata, bulloid pemphigus, chronic eczema, dermatomyositis, erythema nodosum, epidermolysis bullosa, hydradenitis suppurativa, lichen planus, pemphigus vulgaris, psoriasis, pyoderma gangrenosum, scleroderma, or vitiligo.

68. The method of claim 65, wherein the inflammatory disease is sepsis, achalasia, acute or ischemic colitis, acute respiratory distress syndrome, allergy, allograft rejection, alveolitis, Alzheimer's disease, a neurological disease associated with amyloidosis, amebiasis, anaphylactic shock, angiitis, ankylosing spondylitis, appendicitis, arteritis, arthralgia, arthritides, asthma, atherosclerosis, Behcet's syndrome, Berger's disease, bronchiolitis, bronchitis, burns, cachexia, candidiasis, cerebral embolism, cerebral infarction, cholangitis, cholecystitis, chronic fatigue syndrome, celiac disease, Crohn's disease, congestive heart failure, Crohn's disease, cystic fibrosis, Dengue fever, dermatitis, dermatomyositis, disseminated bacteremia, diverticulitis, duodenal ulcers, emphysema, encephalitis, endocarditis, endotoxic shock, enteritis, eosinophilic granuloma, epididymitis, epiglottitis, fasciitis, filariasis, gastric ulcers, Goodpasture's syndrome, gout, graft-versus-host disease, granulomatosis, Guillan-Barre syndrome, hay fever, hepatitis, hepatitis B virus infection, hepatitis C virus infection, herpes infection, HIV infection, Hodgkin's disease, hydatid cysts, hyperpyrexia, immune complex disease, influenza, juvenile idiopathic arthritis, malaria, meningitis, multiple sclerosis, a multiple sclerosis-associated demyelination disease, myasthenia gravis, myocardial ischemia, myocarditis, neuralgia, neuritis, organ ischemia, organ necrosis, osteomyelitis, Paget's disease, pancreatitis, ulcerative pancreatitis, pseudomembranous colitis, pancreatitis, paralysis, peptic ulcers, periarteritis nodosa, pericarditis, periodontal disease, peritonitis, pharyngitis, pleurisy, pneumonitis, pneumonoultramicroscopicsilicovolcanokoniosis, polymyalgia rheumatica, prostatitis, psoriatic arthritis, pseudomembranous Reiter's syndrome, reperfusion injury, respiratory syncytial virus infection, rheumatic fever, rheumatoid arthritis, rhinitis, sarcoidosis, septic abortion, septicemia, sinusitis, forms of cancer having an inflammatory component, spinal cord injury, sunburn, synovitis, systemic lupus erythematosus, systemic lupus erythrocytosis, thrombophlebitis, thyroiditis, Type I diabetes, ulcerative colitis, urethritis, urticaria, uveitis, vaginitis, vasculitis, warts, wheals, or Whipple's disease.

69. The method of claim 68, wherein the inflammatory disease is sepsis.

70. The method of claim 52 and claim 54, wherein the disease of immune dysregulation is secondary induced inflammation.

71. The method of claim 70, wherein the secondary induced inflammation is associated with a bacterial infection, a viral infection, a fungal infection, or a parasitic infection.

72. The method of claim 65, wherein the inflammatory disease is allograft rejection and wherein the composition further comprises an immunosuppressant used to inhibit allograft rejection.

73. The method of claim 65, wherein the inflammatory disease is a xenograft rejection and wherein the composition further comprises an immunosuppressant used to inhibit xenograft rejection.

74. The method of claim 53, where the mammal has a cancer and the alteration of the expression or activity of the gene target increases an innate immune response of the mammal

75. The method of claim 52, wherein the mammal is a human.

76. A method for treating a disease of immune dysregulation in a subject, said method comprising administering to the subject a therapeutically effective amount of a gliptin, benzamil, or ISA2011B.

77. The method of claim 76, wherein the gliptin is vildagliptin, saxagliptin, alogliptin, linagliptin, sitagliptin, gemigliptin, anagliptin, and teneligliptin.

78. The method of claim 77, wherein saxagliptin is administered.

79. The method of claim 76, wherein benzamil is administered.

80. The method of claim 76, wherein ISA2011B is administered.

81. The method of claim 76, wherein the disease of immune dysregulation is an inflammatory disease.

82. The method of claim 81, wherein the inflammatory disease is a dermatological disorder.

83. The method of claim 82, wherein the dermatological disorder is atopic dermatitis, alopecia areata, bulloid pemphigus, chronic eczema, dermatomyositis, erythema nodosum, epidermolysis bullosa, hydradenitis suppurativa, lichen planus, pemphigus vulgaris, psoriasis, pyoderma gangrenosum, scleroderma, or vitiligo.

84. The method of claim 81, wherein the inflammatory disease is sepsis, achalasia, acute or ischemic colitis, acute respiratory distress syndrome, allergy, allograft rejection, alveolitis, Alzheimer's disease, a neurological disease associated with amyloidosis, amebiasis, anaphylactic shock, angiitis, ankylosing spondylitis, appendicitis, arteritis, arthralgia, arthritides, asthma, atherosclerosis, Behcet's syndrome, Berger's disease, bronchiolitis, bronchitis, burns, cachexia, candidiasis, cerebral embolism, cerebral infarction, cholangitis, cholecystitis, chronic fatigue syndrome, celiac disease, Crohn's disease, congestive heart failure, Crohn's disease, cystic fibrosis, Dengue fever, dermatitis, dermatomyositis, disseminated bacteremia, diverticulitis, duodenal ulcers, emphysema, encephalitis, endocarditis, endotoxic shock, enteritis, eosinophilic granuloma, epididymitis, epiglottitis, fasciitis, filariasis, gastric ulcers, Goodpasture's syndrome, gout, graft-versus-host disease, granulomatosis, Guillan-Barre syndrome, hay fever, hepatitis, hepatitis B virus infection, hepatitis C virus infection, herpes infection, HIV infection, Hodgkin's disease, hydatid cysts, hyperpyrexia, immune complex disease, influenza, juvenile idiopathic arthritis, malaria, meningitis, multiple sclerosis, a multiple sclerosis-associated demyelination disease, myasthenia gravis, myocardial ischemia, myocarditis, neuralgia, neuritis, organ ischemia, organ necrosis, osteomyelitis, Paget's disease, pancreatitis, ulcerative pancreatitis, pseudomembranous colitis, pancreatitis, paralysis, peptic ulcers, periarteritis nodosa, pericarditis, periodontal disease, peritonitis, pharyngitis, pleurisy, pneumonitis, pneumonoultramicroscopicsilicovolcanokoniosis, polymyalgia rheumatica, prostatitis, psoriatic arthritis, pseudomembranous Reiter's syndrome, reperfusion injury, respiratory syncytial virus infection, rheumatic fever, rheumatoid arthritis, rhinitis, sarcoidosis, septic abortion, septicemia, sinusitis, forms of cancer having an inflammatory component, spinal cord injury, sunburn, synovitis, systemic lupus erythematosus, systemic lupus erythrocytosis, thrombophlebitis, thyroiditis, Type I diabetes, ulcerative colitis, urethritis, urticaria, uveitis, vaginitis, vasculitis, warts, wheals, or Whipple's disease.

85. The method of claim 84, wherein the inflammatory disease is sepsis.

86. The method of claim 76, wherein the disease of immune dysregulation is secondary induced inflammation.

87. The method of claim 86, wherein the secondary induced inflammation is associated with a bacterial infection, a viral infection, a fungal infection, or a parasitic infection.

88. The method of claim 81, wherein the inflammatory disease is allograft rejection and wherein the composition further comprises an immunosuppressant used to inhibit allograft rejection.

89. The method of claim 81, wherein the inflammatory disease is a xenograft rejection and wherein the composition further comprises an immunosuppressant used to inhibit xenograft rejection.

90. The method of claim 76, where the mammal has a cancer and the alteration of the expression or activity of the gene target increases an innate immune response of the mammal

91. The method of claim 76, wherein the mammal is a human.
